(19) United States
(12) Patent Application Publication
(10) Pub. No.: US 2018/0258381 A1
(43) Pub. Date: Sep. 13, 2018

(54) BIO-LAMINA BIOREACTORS AND METHODS OF MAKING AND USING THE SAME

(71) Applicant: Oregon State University, Corvallis, OR (US)

(72) Inventors: Goran Nadezda Jovanovic, Corvallis, OR (US); Kate Schilke, Corvallis, OR (US); Chris Loeb, Denver, CO (US); Frederick Atadana, Chandler, AZ (US); Davis Weymann, Boise, ID (US)

(73) Assignee: Oregon State University, Corvallis, OR (US)

(21) Appl. No.: 15/981,753

(22) Filed: May 16, 2018

Related U.S. Application Data
(63) Continuation of application No. PCT/US2016/062297, filed on Nov. 16, 2016.
(60) Provisional application No. 62/256,565, filed on Nov. 17, 2015.

(51) Int. Cl. C12M 3/06; C12P 7/04; C12M 1/00; C12M 1/12

(52) U.S. Cl. CPC C12M 23/16; C12P 7/04; C12M 23/20; C12M 25/14; C12M 21/12

(57) ABSTRACT

Disclosed herein are embodiments of bio-lamina bioreactors and methods of making and using the same. The bio-lamina bioreactors disclosed herein can make fuels from organic reactants using fluid flow through the bio-lamina bioreactors in combination with microorganisms capable of reacting with the organic reactants. The microorganisms are embedded within biofilms present within the bio-lamina bioreactor. The bio-lamina bioreactor further comprises bio-lamina substrates comprising unique flow channels and structural projections that facilitate fluid flow and interactions with the microorganism cultures of the biofilm.

### CROSS REFERENCE TO RELATED APPLICATION

This application is a continuation of International Application No. PCT/US2016/062297, filed Nov. 16, 2016, which in turn claims the benefit of and priority to the earlier filing date of U.S. Provisional Patent Application No. 62/256,565, filed on Nov. 17, 2015; each of these prior applications is incorporated herein by reference it its entirety.

### ACKNOWLEDGMENT OF GOVERNMENT SUPPORT

This invention was made with government support under Contract No. DE-AR000439 awarded by the U.S. Department of Energy. The government has certain rights in the invention.

### FIELD

Disclosed herein are embodiments of bioreactor devices capable of converting organic species into fuels using microchannel technology combined with bio-lamina substrates modified with biologically active materials. Also disclosed herein are embodiments of methods for making and using the bioreactor devices.

### BACKGROUND

Creating devices that can provide a sustainable supply of energy/fuel is a central challenge of the 21^{st} century. Catalytically converting different organic species into fuels or energy sources can be used not only provides effective methods for producing useful materials from simple starting materials (which can include waste by-products produced in a variety of industries), but also to reduce the amount of particular organic species that are released into the atmosphere. Conventional methods to produce fuels and energy sources from organic precursors typically require using devices that are not commercially scalable due to cost restrictions, operational restrictions, and yield restrictions. For example, conventional chemostat fermenters typically cannot be produced on the scale needed to achieve suitable fuel amounts in commercial applications. A need exists in the art for a device that can produce commercially-viable amounts of fuels without a corresponding increase in cost and complexity.

### SUMMARY

Disclosed herein are embodiments of a bio-lamina bioreactor, comprising a biofilm bio-lamina substrate comprising one or more structural projections; a biofilm comprising a microorganism, wherein the biofilm is coupled to the bio-lamina substrate; and a fluid flow bio-lamina substrate comprising one or more structural projections, one or more fluid mixers, one or more feed holes, and one or more channel manifolds. In some embodiments, the bio-lamina bioreactor further comprises a first clamp plate and a second clamp plate, an inlet for introducing liquid into the bio-lamina bioreactor, an inlet for introducing gas into the bio-lamina bioreactor, and an outlet for delivering fluid from the bio-lamina bioreactor, or a combination thereof. In some embodiments, the bio-lamina bioreactor comprises two inlets for introducing gas into the bio-lamina bioreactor. The biofilm bio-lamina substrate and the fluid flow bio-lamina substrate can comprise a polymer substrate comprising polycarbonate, polyethylene terephthalate (PET), polyether imide (PEI), poly(methyl methacrylate) (PMMA), poly(tetrafluoroethylene) (PTFE), or a combination thereof. The biofilm bio-lamina substrate and the fluid flow bio-lamina substrate can be a metal substrate comprising a metal selected from stainless steel, copper, titanium, nickel, aluminum, or combinations thereof. In some embodiments, the biofilm has a thickness of 10 μm to 1 mm.

In some embodiments, the biofilm further comprises a film-forming matrix. The film-forming matrix can be formed between a polysaccharide and an inorganic salt. In some embodiments, a polymer can be used alone, or in combination with a polysaccharide. In some embodiments, the polymers can include hydrolyzed polymaleic anhydride, polyvinyl alcohol (PVA), polyacrylic acid (PAA), polycarbonate, or combinations thereof. The polysaccharide can be alginate and the inorganic salt can be CaCl₂. In some embodiments, the film-forming matrix further comprises polylysine, chitosan, adipic dihydrazide, or an aminosilane. The microorganism can be a methanotroph. And in some embodiments, the biofilm comprises a combination of a methanotroph, alginate, and calcium ions. In some embodiments, the biofilm is covalently attached to the biofilm bio-lamina substrate. In yet some additional embodiments, the biofilm is covalently attached to the biofilm bio-lamina substrate through the polylysine, chitosan, adipic dihydrazide, or the aminosilane. In yet some other embodiments, the biofilm is electrostatically coupled to the biofilm bio-lamina substrate.

In some embodiments, the biofilm bio-lamina substrate and the fluid flow bio-lamina substrate comprise a plurality of structural projections. The plurality of structural projections present on the fluid flow bio-lamina substrate can be configured to provide a gradient through which fluid flows. In some embodiments, the plurality of structural projections comprises structural projections of different sizes to form the gradient. The one or more fluid mixers can comprise elevated projections that are configured to provide a tapered flow channel through which liquid can flow. In some embodiments, the feed hole can be located within the tapered flow channel. The one or more channel manifolds each comprise at least one channel and at least one opening through which gas or liquid can be introduced. In some embodiments, fluid flow bio-lamina substrate comprises a plurality of fluid mixers and a plurality of channel manifolds.

In some embodiments, the device can comprise a biofilm bio-lamina substrate comprising a plurality of structural projections and wherein the biofilm bio-lamina substrate is coupled to a biofilm comprising a film-forming material and a microorganism embedded in the film-forming material; a fluid flow bio-lamina substrate comprising a plurality of structural projections configured to align with the plurality of structural projections of the biofilm bio-lamina substrate; a plurality of fluid mixers each comprising a tapered flow channel and a feed hole; a first channel manifold comprising a first opening; and a second channel manifold comprising a second opening; a top clamp plate comprising a plurality of alignment pins; a bottom clamp plate comprising a plurality of alignment holes configured to accept the plurality of alignment pins of the top clamp plate.

Also disclosed herein are embodiments of a biofilm bio-lamina substrate coupled to a biofilm comprising a microorganism, wherein the biofilm bio-lamina substrate comprises one or more structural projections. In some embodiments, the biofilm bio-lamina substrate is covalently or electrostatically coupled to the biofilm.

Also disclosed herein are embodiments of a fluid flow bio-lamina substrate, comprising one or more structural projections, one or more fluid mixers, and one or more channel manifolds. The one or more fluid mixers can comprise elevated projections that are configured to provide a tapered flow channel through which liquid can flow. The fluid flow bio-lamina substrate can further comprise a feed hole positioned with the tapered flow channel. The one or more channel manifolds can each comprise at least one channel and at least one opening through which gas or liquid can be introduced. In some embodiments, the fluid flow bio-lamina substrate comprises a plurality of fluid mixers and a plurality of channel manifolds.

Also disclosed herein are embodiments of a method for making a biofilm bio-lamina substrate. The method can comprise combining a microorganism cell and a polysaccharide to form a biofilm precursor solution; covering at least a portion of a top surface of a bio-lamina substrate comprising one or more structural projection with the biofilm precursor solution to form a biofilm precursor layer; and exposing the biofilm precursor layer to an inorganic salt component to promote crosslinking of the polysaccharide to thereby form a biofilm on the bio-lamina substrate. In some embodiments, the method can further comprise using an internal gelation system to form the biofilm. The internal gelation system can comprise an acid anhydride (such as, but not limited to acetic anhydrides, succinic anhydrides, maleic anhydrides, glutaric anhydrides, and the like), and/or glucono-delta-lactone. In yet additional embodiments, the method further comprises pre-treating the bio-lamina substrate with an organic polymer or linking agent prior to covering the top surface of the bio-lamina substrate with the biofilm precursor solution. The method also can further comprise pre-treating the bio-lamina substrate with polylysine, chitosan, adipic dihydrazide, or an aminosilane.

Also disclosed herein are embodiments of a method of using a bio-lamina bioreactor as disclosed herein. In some embodiments, the method comprises introducing a liquid and at least one organic reactant into a bio-lamina bioreactor comprising a biofilm bio-lamina substrate comprising one or more structural projections and coupled to a biofilm comprising a microorganism; a fluid flow bio-lamina substrate comprising one or more structural projections, one or more fluid mixers, and one or more channel manifolds; a top clamp plate; and a bottom clamp plate; and isolating a fuel produced by reaction of the organic reactants with the microorganism that is expelled from the bio-lamina bioreactor. In some embodiments, the liquid is water and the at least one organic reactant is a gas. The gas can be selected from methane, oxygen, and combinations thereof. In some embodiments, the liquid is introduced into the bio-lamina bioreactor at a rate of greater than 0 mL/hr to 500 mL/hr and the organic reactant is introduced into the bio-lamina bioreactor at a rate of greater than 0 mL/hr to 5,000 mL/hr at 1 atm. In yet additional embodiments, the method comprises introducing a first organic reactant into the bio-lamina bioreactor and introducing a second organic reactant into the bio-lamina bioreactor.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 is a schematic diagram of an exemplary method of producing a film-forming matrix using a polysaccharide and an inorganic salt component.

FIGS. 2A and 2B are schematic diagrams illustrating of a method of producing a biofilm embodiment on a bio-lamina substrate (FIG. 2A) and the reactions involved in a representative internal gelation method (FIG. 2B).

FIG. 3 is a schematic diagram of another exemplary method of producing a film-forming matrix using a combination of a polymer, a polysaccharide, and an inorganic salt component.

FIG. 4 is a schematic diagram of yet another exemplary embodiment of a method of producing a film-forming matrix using an aminosilane to facilitate binding of the film-forming matrix to a bio-lamina substrate.

FIG. 5 is a schematic diagram of an exemplary embodiment of a method of producing encapsulated microorganisms in a biofilm using photo-induced polymerization.

FIG. 6 is a schematic diagram of an exemplary embodiment of a biofilm produced using nanosprings.

FIG. 7 is a top perspective view of an exemplary biofilm bio-lamina substrate.

FIG. 8 is a micrograph image of structural projections etched into the surface of the bio-lamina plate.

FIGS. 9A-9C are diagrams illustrating a variety of representative structural projections having different types of gradients; FIG. 9A depicts structural projections enhanced with gradient coatings that change contact angle; FIG. 9B illustrates structural projections that are exposed to local changes in temperature and/or surfactant concentrations, thus creating local surface tension gradients; and FIG. 9C illustrates structural projections that create a gradient due to size and/or curvature differences between the structural projections.

FIG. 10 is an image illustrating flow through a bio-lamina comprising structural projections exposed to local change in temperature or surfactant concentration, thus creating local surface tension gradients.

FIG. 11 is an image illustrating the organizations of structural projections in an exemplary bio-lamina substrate.

FIG. 12 is a photographic image of an exemplary biofilm bio-lamina substrate coated with a biofilm.

FIG. 13 is a top perspective view of an exemplary fluid flow bio-lamina substrate.

FIGS. 14A and 14B are top plan views of portions of a fluid flow bio-lamina substrate; FIG. 14A illustrates a portion of a fluid flow bio-lamina comprising a plurality of fluid mixers and a plurality of channel manifolds; FIG. 14B illustrates an exemplary channel manifold configuration comprising a variety of openings through which fluids can be introduced into the bio-lamina bioreactor.

FIG. 15 is a top plan view of an exemplary fluid mixer component through which liquid flows to enter the portion of the fluid flow bio-lamina comprising a plurality of structural projections.

FIG. 16 is top perspective view of an exemplary fluid mixer component comprising a mixing chamber through which liquid flows so as to facilitate mixing with gas introduced through a feed hole.

FIG. 17 is a top plan view of a photomicrograph showing an exemplary fluid mixer component comprising a mixing chamber, a flow-through channel and a gas feed hole positioned within the flow-through channel.

FIG. 18 is a perspective view of an exemplary constructed bio-lamina bioreactor.

FIG. 19 is an exploded perspective view of the exemplary bio-lamina bioreactor illustrated in FIG. 18, which illustrates the various components of the constructed bio-lamina bioreactor.

FIG. 20 is a perspective view of an exemplary bio-lamina bioreactor set-up illustrating various exemplary components that can be used in combination with the bio-lamina bioreactor during use.

FIG. 21 is a schematic diagram of an exemplary bio-lamina bioreactor set-up illustrating the various connections between the components of the set-up.

FIG. 22 is perspective view of an exemplary bio-lamina bioreactor device as it is connected within an exemplary set-up for use.

FIG. 23 is a schematic cross-sectional view of a fluid flow bio-lamina/biofilm bio-lamina combination wherein a flow channel is formed between the fluid flow bio-lamina and the biofilm bio-lamina and further illustrating transport of gases from a gas bubble into the biofilm as the gas bubble flows through the flow channel.

FIG. 24 is an image of live cells in an exemplary biofilm imaged using live/dead staining technique.

FIG. 25 is a graph of reaction rate as a function of aqueous methane concentration illustrating results obtained from analyzing the rate of methane consumption in an immobilized biofilm.

FIG. 26 is a graph of methane conversion as a function of residence time illustrating competitive inhibition results.

FIG. 27 is a graph of cyclopropane conversion as a function of residence time illustrating results obtained from inhibitor studies using a packed bed reactor.

FIG. 28 is a bar graph of methane conversion (2800), methanol selectivity (2802), and cyclopropane conversion (2804) as a function of residence time.

FIG. 29 is an illustration of a drip flow reactor device that can be used for biofilm growth in certain embodiments wherein biofilm viability is tested.

FIG. 30 is bar chart illustrating results obtained from shear strength testing of different embodiments of film-forming matrices.

FIG. 31 is a graph of bed depth as a function of time illustrating results obtained from analysis of biofilm integrity using different stabilizing components, such as phosphate and HEPES buffer.

FIG. 32 is a graph illustrating microorganism growth in a chemostat used to produce representative microorganism cells used in embodiments of the bio-lamina bioreactors disclosed herein.

FIGS. 33A and 33B are graphs illustrating results obtained from analysis of the microorganism cultures produced in embodiments described herein; FIG. 33A is a graph illustrating methanotrophic biomass concentration in a chemostat over a pseudo-steady-state operational period and FIG. 33B is a graph of fluid flow and aqueous volume in the chemostat over time.

FIGS. 34A and 34B are graphs illustrating results obtained from analysis of the microorganism cultures produced in embodiments described herein; FIG. 34A is a graph of the range of chemostat solids retention times during a pseudo-steady state operational period; and FIG. 34B is a graph of measured aqueous methane concentration and equilibrium aqueous methane concentration calculated from measured effluent gas methane concentration.

FIGS. 35A and 35B are graphs of chemostat methanol concentration over time; FIG. 35A illustrates methanol production measured over days and FIG. 35B illustrates methanol production measured over hours.

FIGS. 36A-36D illustrate results obtained from cyclopropanol production and inhibition of methanol dehydrogenase (MDH) of microorganisms encapsulated in 2-3 mm calcium alginate beads; FIG. 36A is a gas chromatogram illustrating production of M. trichosporium OB3b in alginate beads incubated for 18 hours with cyclopropane (peaks 1, 2, and 3), which produced cyclopropanol (peaks 4, 5 and 6); FIG. 36B is a bar graph illustrating cyclopropane and cyclopropanol content of the alginate bead mixture in the absence of methane; FIG. 36C is a graph of methanol production after the beads were incubated with methane; and FIG. 36D illustrates the inhibitor effect of cyclopropanol on methanol consumption of fresh microorganism cultures.

FIGS. 37A-37C are graphs illustrating the effects of cyclopropane on microorganisms in alginate beads after different exposure times; FIG. 37A illustrates methanol concentration after two hours of exposure to cyclopropane; FIG. 37B illustrates methanol concentration after six hours of exposure to cyclopropane; and FIG. 37C illustrates methanol concentration after 18 hours of exposure to cyclopropane.

FIG. 38 is a bar graph of response of rates of methane consumption and methanol production in a column packed with a representative microorganism culture immobilized in alginate beads wherein the arrows indicate addition of cyclopropanol to inhibit MDH activity.

FIGS. 39A and 39B illustrates results obtained from analysis of a representative microorganism culture after ethylene oxidation (FIG. 39A) and a specific oxygen uptake rate test (SOUR) (FIG. 39B).

FIG. 40 is graph of culture growth in a chemostat during an initial start-up period and after 45 days of semi-stable operation.

FIG. 41 is a graph of methane and oxygen concentrations and culture density of the same culture as that used to obtain the results illustrated in FIG. 40.

FIG. 42 is a graph of methanol production from two different embodiments wherein formate was added to the chemostat housing the microorganism culture being analyzed.

FIG. 43 is a graph illustrating methanol production after addition of varying concentrations of cyclopropanol.

FIGS. 44A and 44B are graphs illustrating methane SOUR data obtained from analysis of the two different embodiments described for FIG. 42 FIGS. 45A and 45B are graphs of methane consumption; FIG. 45A is a graph of methane consumption as a function of residence time and FIG. 45B is a graph of methane consumption and methanol production as a function of time.

FIGS. 46A-46C are graphs of results obtained from operating an exemplary bio-lamina bioreactor as disclosed herein; FIG. 46A is a graph of methanol production as a function of time; FIG. 46B is a graph of cumulative methanol production as a function of time; and FIG. 46C is a graph of carbon conversion efficiency as a function of time.

FIGS. 47A-47C illustrate model diagrams and simulation results obtained from operational models of a bio-lamina bioreactor; FIG. 47A illustrates a single model segment with a gas bubble, wherein the fluid and biofilm areas are modeled; FIG. 47B is a graph of concentration (methanol, oxygen, and methane) as a function of time generated from the modeling; and FIG. 47C are single model 1 cm segments used in modeling.

FIG. 48 is a graph of methanol production as a function of time illustrating the differences in methanol production of an exemplary bio-lamina bioreactor embodiment, a beaded column embodiment, and a chemostat.

FIG. 49 is a bar graph of methane consumption and methanol production as a function of time illustrating results obtained from an exemplary bio-lamina bioreactor embodiment.

FIGS. 50A-50E illustrate an embodiment of a disc-shaped bio-lamina bioreactor; FIG. 50A illustrates a perspective view of a disc-shaped bio-lamina bioreactor; FIG. 50B is an exploded view of the embodiment of FIG. 50A; FIG. 50C illustrates a disc-shaped biofilm bio-lamina substrate; FIG. 50D illustrates one side of a disc-shaped fluid flow bio-lamina substrate; and FIG. 50E illustrates the opposite side of the disc-shaped fluid flow bio-lamina substrate of FIG. 50D.

FIG. 51 is a schematic illustration of an embodiment used to make a polyvinyl alcohol-based biofilm as described herein.

FIG. 52 is a schematic illustration of an embodiment used to make a polyvinyl alcohol-based biofilm in combination with coupling the polyvinyl alcohol-based biofilm to a surface-modified biofilm substrate as described herein.

FIG. 53 is schematic illustration showing an exemplary embodiment of surface-modifying a biofilm substrate.

FIG. 54 includes graphs illustrating results obtained from testing the adhesion strength of exemplary biofilms and corresponding substrates upon which the biofilms are coupled.

FIG. 55 is a graph showing results for methane oxidation behavior of Methylmicrobium buryatense 5G in combination with media, agar, and a polyvinyl alcohol biofilm.

### DETAILED DESCRIPTION

### I. Explanation of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed devices, materials, and methods can be used in conjunction with other devices, materials, and methods. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

To facilitate review of the various embodiments of the disclosure, the following explanations of specific terms are provided:

Bio-Lamina Bioreactor: A reactor comprising at least two bio-lamina substrates, wherein at least one bio-lamina substrate comprises a biofilm and at least one bio-lamina substrate comprises structural features that facilitate deliver and flow of fluids into and through the bioreactor.

Biofilm: A film used to cover, or substantially cover (e.g., 50% to 99%, such as 60% to 99%, or 70% to 90% of surface area), the top surface of a bio-lamina substrate, particularly a biofilm bio-lamina substrate. The biofilm comprises cells of at least one microorganisms and further comprises either film-forming material, a nanomaterial, a natural or synthetic organic polymer or linking agent, or an organic polymer matrix, which are described herein.

Elevated Projection: A portion of a fluid flow bio-lamina that extends from a top planar surface of the fluid flow bio-lamina substrate and that has a shape sufficient to produce a fluid mixer component of the fluid flow bio-lamina substrate.

Fluid Mixer: A portion of a fluid flow bio-lamina that is provided by elevated projections present on the fluid flow bio-lamina, wherein one or more elevated projections are positioned so as to provide a flow channel through which fast flowing liquid can flow to break-up gas flow introduced into the fluid flow bio-lamina through feed holes and thereby to form bubbles.

Structural Projection: A portion of a bio-lamina substrate that extends from a top planar surface of a bio-lamina substrate so as to increase the surface area of the bio-lamina substrate and/or provide mechanical stability for an attached biofilm.

### II. Introduction

Disclosed herein are embodiments of bio-lamina bioreactors that address deficiencies of conventional bioreactors used for methane conversion. Conventional bioreactors, such as chemostats typically utilize submerged microorganism cultures that freely float in liquid. Such conventional bioreactors exhibit a multitude of deficiencies that reduce their use in industry, such as low reactant solubilities, excessive mass transfer resistance due to thick substrate films, and low biomass loading.

The bio-lamina bioreactor embodiments disclosed herein are able to improve reactant solubility during use, reduce mass transfer resistance, and achieve high concentrations of biomass loading within the biofilms used in the bio-lamina bioreactors. For example, the disclosed devices produce high mass transfer rates for supplying nutrients and removing products and toxins through mass transfer areas formed between the biofilm surface and gas bubbles passing through flow channels of the bioreactor and that interact with an immobilized biofilm in the bio-lamina bioreactor. In some embodiments, the disclosed devices are capable of very short diffusion times. Some exemplary embodiments are capable of short total diffusion times, such as 500 ms. The disclosed bio-lamina bioreactors also exhibit high heat transfer rates from the biofilm to interleaved heat exchange microchannels, thereby maintaining optimal conditions for cell viability and productivity. The disclosed bio-lamina bioreactors also have a high bioreactor surface to volume ratio, with some embodiments having ratios on the order of 2×10⁴ m² to 5×10⁴ m² interface surface area per m³ reactor volume. High specific biomass loading also can be obtained with the disclosed bio-lamina bioreactor embodiments, with some providing as high (or even higher) as 50 kg of biomass (within the biofilm) per m³ reactor volume. The design of the disclosed bio-lamina bioreactors also provides the option of stacked plate assembly, which is readily scalable to meet industrial production capacities. Also, the disclosed bio-lamina bioreactors utilize inexpensive construction materials (polymers, glass, stainless steel, etc.) and can be fabricated using facile fabrication techniques (lamination, extruding, thermal embossing, punching, etc.), thereby lending to their scalability and applicability in industry.

### III. Components and Bio-Lamina Bioreactors

Bio-lamina bioreactor embodiments disclosed herein comprise a biofilm containing microorganisms capable of converting various organic species into fuels or other products. The bio-lamina bioreactors comprise unique bio-lamina substrates that are configured to support the biofilm and provide fluidic channels through which the organic species can flow in solution. The bio-lamina substrates utilize unique flow channel configurations, dimensions, and structural features to provide improved diffusion times to deliver nutrients to microorganisms present in the bio-lamina bioreactor. The bio-lamina bioreactors can further comprise various mechanical components that facilitate use, such as clamp plates, suitable inlet and outlet ports, additional components to seal the bio-lamina bioreactor to prevent leakage, and various other additional components. The bio-lamina bioreactor components are described in more detail below.

The biofilms used in the disclosed bio-lamina bioreactors comprises one or more microorganism species capable of converting organic species and gases into fuel. Any microorganism capable of converting an organic compound into a fuel or other by-product can be used. In some embodiments, methanotrophs can be used; however, the device embodiments disclosed herein are not limited to use with methanotrophs and other suitable microorganisms can be used. Exemplary microorganism species can be selected from, but are not limited to, Methylosinus trichosporium, Methylophilus methylotrophus, Methylobaceterium extorquens AM1, Methylosinus trichosporium OB3b, Methyomicrobium burytense, Methylococcus capslatus, Mycobacterium strains JS622, JS623, JS624, JS625, Mycobacterium strains TA5 and TA27, Mycobacterium vaccae JOB5, Rhodococcus rhodochrous, Rhodococcus sp. Strain Sm-1, Xanthobacter Strain Py2, Rhodococcus sp. Strain AD45, Pseudomonas (e.g., Pseudomonas butanavora, Pseudomonas putida, Pseudomonas mendocina), Thauera butanivorans, Burkholderia cepacia G4, Rhodococcus sp. L4, Rhodococcus Ralstonia, Nitrosomonas europae, Providencia alcalifaciens, Bacillus megaterium, Acinetobacter calcoaceticus, Thermobifida fusca, Escherichia coli, Comamonas sp., or combinations thereof.

The biofilm further comprises a film-forming material that comprises a polysaccharide, an inorganic salt, and combinations thereof. Suitable polysaccharides can include, but are not limited to, carboxy- or sulfate-containing polysaccharides. Such polysaccharides include, but are not limited to, alginic acid (or alginate), carboxymethyl cellulose, pectic polysaccharides, carboxymethyl dextran, xanthan gum, carboxymethyl starch, hyaluronic acid, dextran sulfate, pentosan polysulfate, carrageenans, fuciodans, or a combination of two or more thereof. In some embodiments, the polysaccharide can be modified to increase the solubility of methane and oxygen within the biofilm. In some embodiments, the polysaccharide can be modified with perfluorocarbon functional groups and/or surfactants. The polysaccharide may also be chemically modified and covalently cross-linked to stabilize the gel against leaching of ionic species (e.g. Ca²⁺). In some embodiments, the polysaccharide may be activated for crosslinking using carbonyldiimidazole, carbodiimides (with or without esterification with derivatives of nitrophenol, N-hydroxysuccinimide, or hydroxybenzotriazole), phosphoronium compounds, isocyanates, epoxides, or other similar chemical modifications. In some embodiments, the carbohydrate backbone of the alginate polymer can be oxidized with aqueous sodium periodate, to create aldehydes. The modified polymer chains resulting from any of the above modifications can then be cross-linked using a polyamine (e.g., ethylenediamine, bis-aminopolyethylene glycol, or polymers, such as chitosan, polylysine or polyallylamine), or a dihydrazide (e.g., adipic dihydrazide). Other embodiments may use variants of “click” chemistry (such as using azides and alkynes) or photoreactive cross-linkers (such as benzophenone derivatives) to chemically cross-link the gel. In some embodiments, a polymer can be used alone, or in combination with a polysaccharide. Suitable polymers include, but are not limited to, hydrolyzed polymaleic anhydride, polyvinyl alcohol (PVA), polyacrylic acid (PAA), polycarbonate, or combinations thereof.

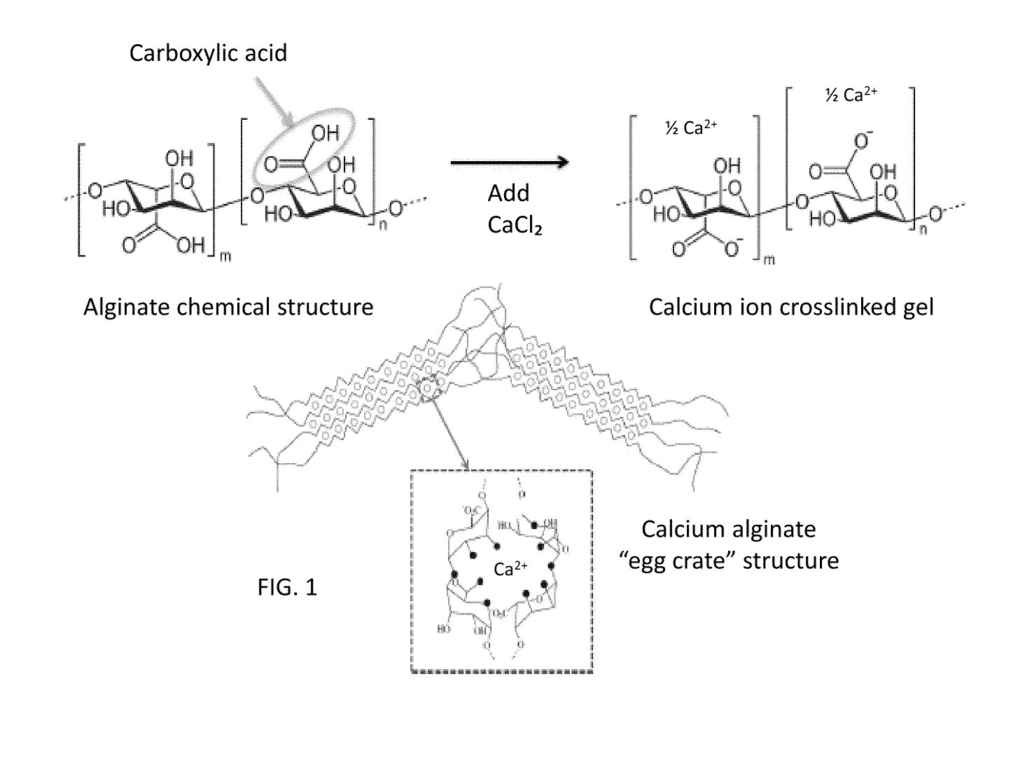

The inorganic salt component can comprise at least one monovalent or multivalent (such as divalent, trivalent, or tetravalent) ion and a suitable counter-ion. In some embodiments, the inorganic salt compound can be selected from a sodium-containing salt, a potassium-containing salt, a calcium-containing salt, a magnesium-containing salt, a tin-containing salt, or a combination thereof. Such salts can comprise any suitable counter-ion, such as halogens, acetates, silicates, carbonates, or combinations thereof. Suitable inorganic salt components can be selected from calcium salts (e.g., calcium chloride, calcium acetate, and the like), tin salts (e.g., stannous chloride and the like), sodium salts (e.g., sodium chloride, sodium acetate, and the like), magnesium salts (e.g., magnesium chloride and the like), potassium salts (e.g., potassium chloride, potassium iodide, and the like), boric acid salts, sulfuric acid salts, phosphoric acid salts, salts comprising iron (Fe²⁺ and/or Fe³⁺), aluminum (Al³⁺), barium (Ba²⁺), strontium (Sr²⁺), magnesium (Mg²⁺), manganese (Mn²⁺), or combinations thereof. The choice of inorganic salt components can depend on possible metabolic interference or toxicity of these components towards immobilized microorganisms. In exemplary embodiments, the biofilm is formed from a composition comprising alginate, calcium chloride, and one or more microorganisms. An exemplary scheme illustrating formation of a film-forming material is illustrated in FIG. 1. In yet additional embodiments, the biofilm can be formed from a composition comprising a polymer as described above (e.g., hydrolyzed polymaleic anhydride, polyvinyl alcohol (PVA), polyacrylic acid (PAA), polycarbonate, or combinations thereof) and one or more boric acid salts. Such compositions can further comprise salts of sulfuric and/or phosphoric acid (e.g., sodium salts of such acids). These additional sulfuric and/or phosphoric acid salts can contribute additional biofilm stabilization. An exemplary embodiment comprises a combination of a sodium boric acid salt and PVA, which can then be modified with sodium sulfate to cause displacement and augmentation of the borate-PVA bonds with sulfate bonds, which exhibit augmented stability. Additional substitution with sodium phosphate results in stable phosphorylation, which increases hydrophobicity of the polymers and prevents their dissolution in aqueous media.

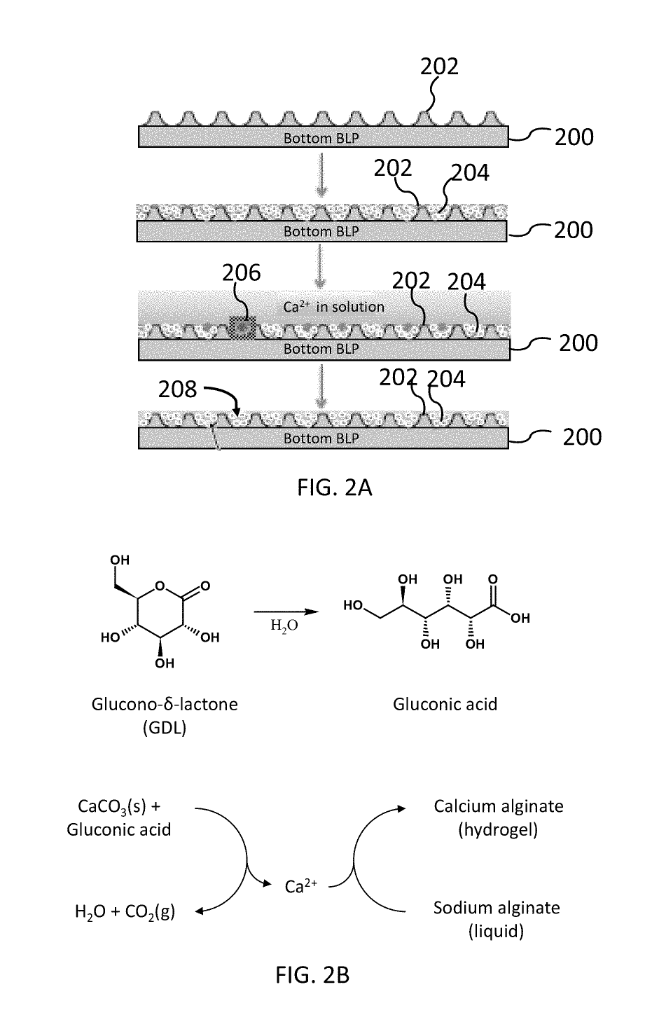

In some embodiments, the biofilm can be formed by exposing cells of the microorganism to a solution of the polysaccharide. The resulting mixture can then be exposed to the inorganic salt component to form a film-forming material with the polysaccharide, thereby forming a matrix comprising microorganism cells. By oxidizing the bio-lamina substrate (e.g., such as by hot oxidation of steel at temperatures of 800° C. or higher), iron, nickel, and chromium surface ions can be produced in contact with film-forming material and cells contained therein to act as a molecular primer to anchor the biofilm comprising the film-forming material and microorganisms to a bio-lamina substrate, such as a metal bio-lamina. A schematic diagram of an exemplary embodiment of this process is illustrated in FIG. 2A. As illustrated in FIG. 2A, a bio-lamina substrate 200 comprising a plurality of structural projections 202 is exposed to a solution of the polysaccharide and microorganisms (illustrated as 204). The microorganism cells can be provided by the solution, or they can be spread or attached onto the polysaccharide layer after it is formed. A solution of the inorganic salt component can then be added to thereby deposit ionic species 206 within the polysaccharide/microorganism layer. After crosslinking of the polysaccharide with the ionic species, biofilm 208 is produced. In some embodiments, uniformity of the gel layer can be improved by first spraying the surface with a cross-linking solution (e.g., a CaCl₂ solution) to promote setting of the top layer of the gel prior to immersion in a cross-linking solution.

In some embodiments, an “internal gelation” system can be used to obtain the film-forming material of the biofilm. The internal gelation system uses a time-release system (e.g., a combination of an acid anhydride (such as, but not limited to acetic anhydrides, succinic anhydrides, maleic anhydrides, glutaric anhydrides, and the like), glucono-delta-lactone, or a combination thereof, and an inorganic salt component) to provide free positively charged polyvalent ions to crosslink the polysaccharide matrix. These embodiments can provide improved uniform films as compared to those obtained using methods wherein the polysaccharide is merely immersed in a solution of the inorganic salt component. The internal gelation system also can be used to formulate film-forming material suspensions, which can be cast into films, including thick films, thin films, and films having various shapes. The internal gelation method can be used to replace the conventional immersion methods, which can lead to a tough outer coating formed over a softer, weaker bulk film due to rapid hardening of the film surface. In contrast, the disclosed internal gelation method utilizes the relatively slow dissolution of Ca²⁺-rich microparticles dispersed in the bulk polysaccharide, thus producing more uniform film-forming matrices.

In one embodiment, calcium carbonate and/or calcium sulfate powder (0.1% to 1% w/v) is added to a 4 wt % aqueous alginate solution. A slight excess (approximately 2× vs. CaCO₃) of glucono-δ-lactone is dissolved in the cell suspension, and this is immediately mixed with the alginate. Hydrolysis of the lactone cyclic ester results in production of gluconic acid, which dissociates the calcium carbonate into Ca²⁺ ions and CO₂. These divalent ions then crosslink the alginate chains, producing a uniform solid gel in the shape of its container. Gelation times can be modified substantially by changing the temperature, initial pH, concentrations of alginate and crosslinking compounds, etc. The surface of the resulting biofilm can be further stabilized through increased crosslinking by immersion in 0.1 to 0.5 M CaCl₂ for 5 to 30 minutes. A representative schematic is illustrated in FIG. 2B.

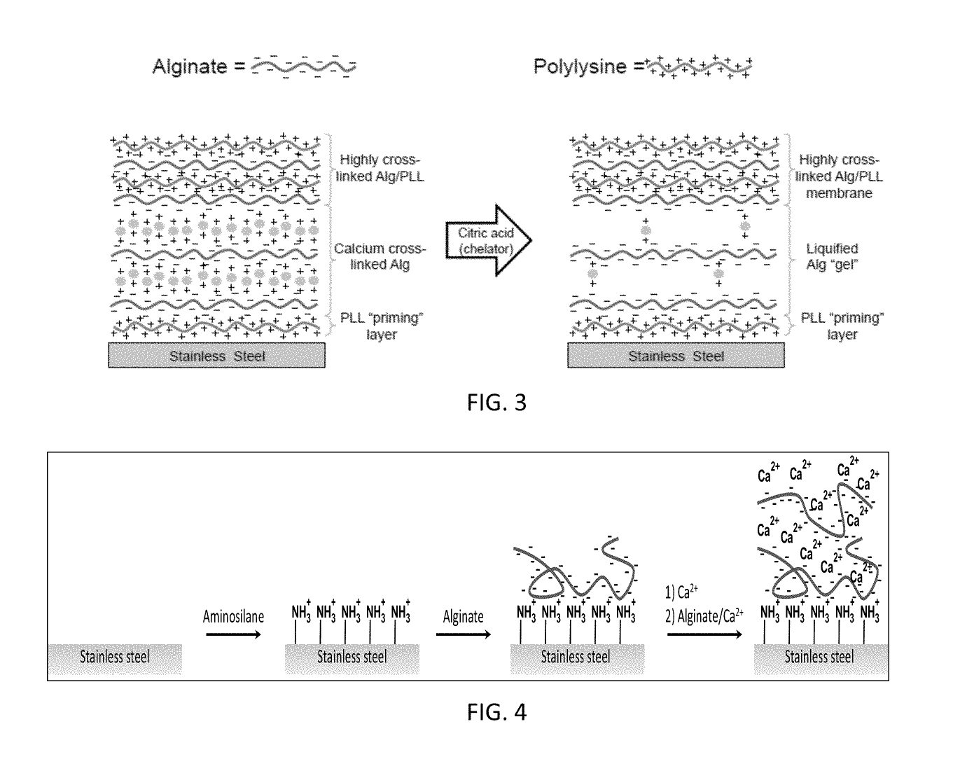

Other embodiments of biofilms can be used in the disclosed bio-lamina bioreactors. Other such biofilms can include biofilms made using film-forming materials, such as those described above, and a surface treatment of the bio-lamina substrate with one or more organic polymers and/or linking agents, such as (but not limited to) polylysine, chitosan, adipic dihydrazide, or an aminosilane. Biofilms using these components can be formed by first depositing a surface modification layer of the organic polymer or the linking agent, followed by a layer of the film-forming material and then another layer of the organic polymer or linking agent mixed with a polysaccharide (which can be the same or different from the polysaccharide of the film-forming material). Addition of a chelator (e.g., citric acid, phosphate, EDTA, or combinations thereof) can be used to convert the film-forming material layer into a film. An exemplary schematic illustration of this type of biofilm and its formation is illustrated in FIG. 3.

Without being limited to a single theory of operation, it is currently believed that chemical modification of bio-lamina surface with the organic polymer or aminosilane will impart a high positive surface charge at neutral or mildly acidic pH. Electrostatic interactions between the positively-charged surface and the negatively-charged polysaccharide components greatly enhance adhesion of the biofilm. Also, in some embodiments, the first layer of the polysaccharide can be covalently coupled to the surface-modified amine groups thereby covalently attaching the biofilm to the bio-lamina surface. An exemplary schematic illustration of such embodiments is illustrated in FIG. 4. In some embodiments, the polysaccharide can be modified with amine-reactive NHS esters to facilitate coupling of the polysaccharide to the amine groups. In other embodiments, hydroxyl groups of the polysaccharide can be oxidized to amine-reactive aldehyde functional groups. The hydrazone products formed by the reaction of amines and aldehydes may optionally be further stabilized by treatment with a chemical reducing agent, such as sodium borohydride, sodium cyanoborohydride, or the like.

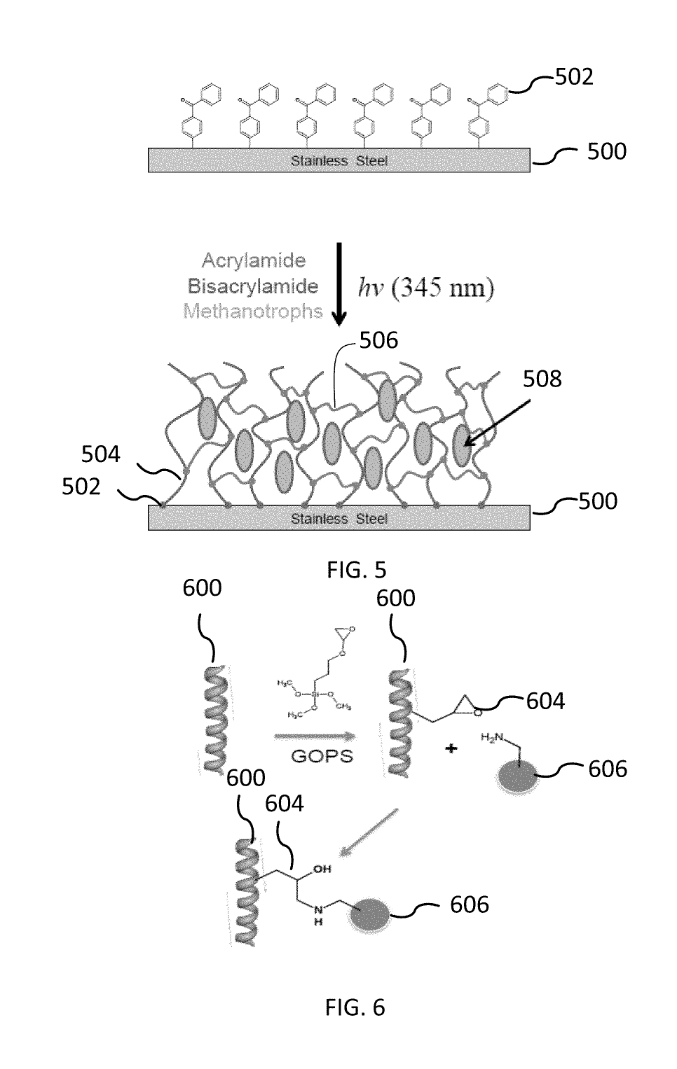

In yet additional embodiments, the biofilm can comprise an organic polymer matrix comprising encapsulated microorganism cells. In such biofilm embodiments, the organic polymer matrix can be formed by exposing a surface-modified lamina to one or more organic compounds capable of forming covalent bonds with the surface-modified lamina. Solely by way of example, the surface-modified lamina can comprise a plurality of benzophenone molecules covalently attached to a lamina surface. Such surface-modified lamina need not be limited to covalently attached benzophenone molecules as other suitable compounds can be used as long as they comprise one or more functional groups (e.g., a carbonyl) that can react with the one or more organic compounds of the organic polymer matrix. In some embodiments, the one or more organic compounds are selected from amine-containing compounds, thiol-containing compounds, or hydroxyl-containing compounds comprising one or more sites of unsaturation. In particular disclosed embodiments a combination of organic compounds comprising one or more sites of unsaturation can be used to form a cross-linked matrix after exposure to an energy source capable of producing energy sufficient to initiate cross-linking between sites of unsaturation present in the organic compounds. In exemplary embodiments, the one or more organic compounds can be selected from acrylamide, bisacrylamide, acrylate, thiol acrylate, and combinations thereof. In particular disclosed embodiments, acrylamide and bisacrylamide are used to form the polymer matrix by combining the acrylamide, bisacrylamide, and the microorganism cells with the surface-modified lamina and then using a light source to initiate crosslinking and organic polymer matrix formation with encapsulated microorganism cells. An exemplary schematic illustration of the formation of such an organic polymer matrix is illustrated in FIG. 5. As illustrated in FIG. 5, benzophenone compounds 502 can be linked to substrate 500 and then acylamide linkers 504 can be used to bind to the benzophenone compounds 502. Methanotrophs 508 can be contained in a cross-linked matrix between acrylamide linkers 504 and bisacrylamide linkers 506.

In additional embodiments, a substrate surface can be surface-modified to promote increased biofilm layer formation on the substrate. In some embodiments, a substrate can be surface-modified by reacting a surface of the substrate (e.g., a metal oxide substrate, a stainless steel substrate, a glass substrate, an aluminum substrate, and the like) with glycidylpropoxytrimethoxysilane (GPTMS), or derivatives thereof, followed by tris(hydroxymethyl)aminomethane (Tris), or derivatives thereof, to produce a hydroxyl-rich surface coating that interact with a biofilm composition, such as those described herein. The biofilm composition can be thus be covalently anchored to the surface-modified substrate. In some embodiments, the substrate can be first be surface modified with a layer of an aminopropyltrialkoxysilane (such as aminopropyltrimethoxysilane and/or aminopropyltriethoxysilane). This can form a glass-like layer on the substrate, which can further interact with a GPTMS/Tris conjugate to form a surface-modified substrate capable of covalently anchoring the biofilm.

In some embodiments, the biofilm can comprise microorganism cells immobilized on a nanomaterial present on a lamina surface. For example, nanosprings can be grown onto or coupled to a lamina surface using techniques known to those of ordinary skill in the art. The deposited nanosprings can be modified with an epoxy-containing compound, such as glycidoxypropyltriethoxysilane, to provide an epoxy-modified nanospring. The epoxy-modified nanospring can then react with functional groups (e.g., amines, hydroxyl groups, thiols) present in the microorganism cell's structure to immobilize the microorganism cell on the lamina. An exemplary schematic illustration of a method of making this type of biofilm is illustrated in FIG. 6. FIG. 6 illustrates a nanospring 600 that can be coupled to an epoxy-containing compound 604. Upon addition of compound 606, which comprises a microorganism cell, the nanospring 600 can be coupled to the microorganism cell 606 through linker 604. Other supports for the biofilm can include electrospun polymer fibers, functionalized fiberglass or organic fiber mats, or combinations thereof.

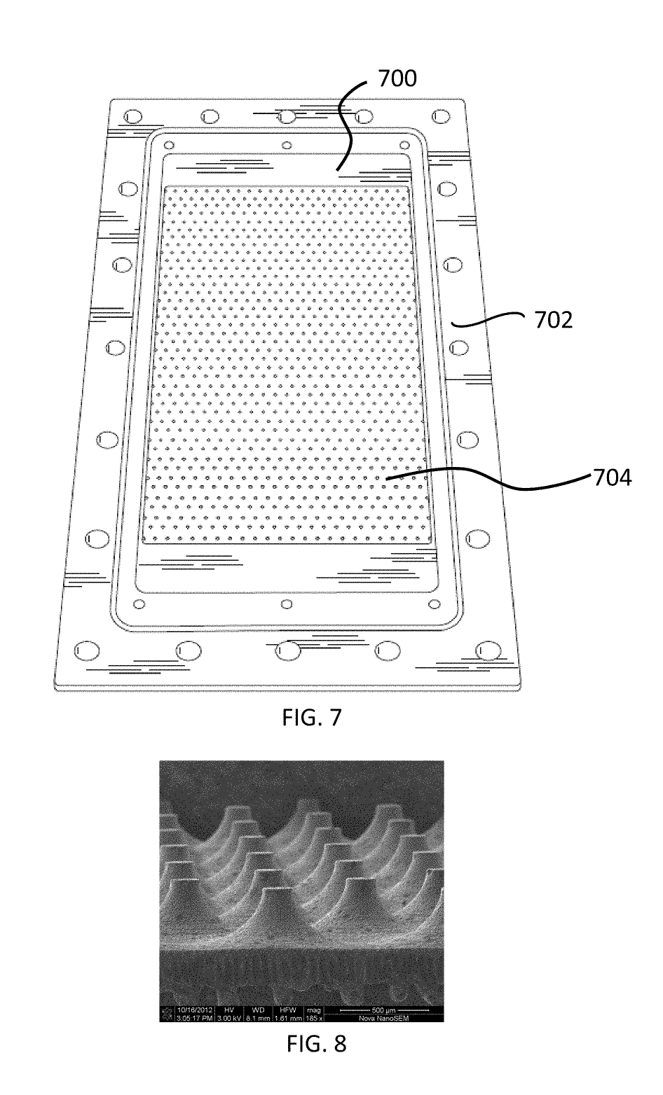

The bio-lamina bioreactors disclosed herein also comprise at least two bio-lamina substrates, one of which is coupled to a biofilm (referred to herein as a biofilm bio-lamina substrate) as described above and one of which is used for fluid flow (referred to herein as a fluid flow bio-lamina substrate). Each of the biofilm bio-lamina and fluid flow bio-lamina substrates comprises a top surface and a bottom surface, wherein the top surface comprises a plurality of structural projections that extend from the top surface. The structural projections can have any size and shape. In some embodiments, the structural projections can have heights ranging from greater than 0 nm to 1×10⁶ nm, or 5 μm to 5,000 μm, such as 20 μm to 1,000 μm, or 50 μm to 350 μm as measured from the top surface of the bio-lamina to the top of the structural projection. In some embodiments, the structural projections can have heights ranging from greater than 0 μm to 1,000 μm, such as 100 μm to 750 μm, or 200 μm to 500 μm. In an exemplary embodiment, the structural protections had a height of 460 μm or 360 μm. FIG. 7 provides a top perspective view of a top surface of a biofilm bio-lamina substrate 700 that is associated with a bottom clamp plate 702. Biofilm bio-lamina substrate 700 comprises a plurality of structural projections 704.

The structural projections are selected to have any shape that enhances the surface area of the bio-lamina substrate. In some embodiments, the structural projections can be shaped as tapered projections, cylindrical projections, half-sphere projections, non-symmetrical projections, or combinations thereof. In some embodiments, the structural projections are cylindrical, or substantially cylindrical and have a diameter ranging from greater than 0 mm to 10 mm, such as 0.001 mm to 10 mm, or 0.01 mm to 10 mm, or 0.1 mm to 10 mm. In some embodiments, cylindrical or substantially cylindrical structural projections had diameters ranging from 1 mm to 5 mm, such as 1 mm to 4 mm, with some embodiments being 1 mm, 2 mm, or 3 mm in diameter. Any number of structural projections can be included on the top surface of the biofilm bio-lamina and fluid flow bio-lamina substrates. In particular disclosed embodiments, the number of structural projections present on the biofilm bio-lamina substrate is equal to that of the fluid flow bio-lamina substrate. In other embodiments, the number of structural projections present on the biofilm bio-lamina and fluid flow bio-lamina substrates can be different. Fluid flow bio-lamina substrates also can contain structural projections to provide directionality of fluid flow for both the gaseous phase (bubbles) and the liquid phase. In addition, some of the structural projections of the fluid-flow bio-lamina can meet (or touch) the structural projections on the biofilm bio-lamina substrate, thus providing exact spatial distance between biofilm bio-lamina substrate and fluid flow bio-lamina substrate. Exemplary structural projections are illustrated in FIG. 8.

In yet additional embodiments, the structural projections can be patterned onto the biofilm bio-lamina and fluid flow bio-lamina substrates so as to provide a gradient of structural projections. In some embodiments, the height of the structural projection can be varied so as to provide a gradient based on structural projection height. In yet additional embodiments, the number of structural projections can be varied so as to provide a gradient. In yet other embodiments, both the number and height of the structural projections can be varied. In general, the gradient of a field, or the gradient of energy potential (universal chemical potential) creates force. Thus, gradient properties of the structural projections can result in gradient potential energy for any or all fluids in multiphase flow providing that these properties are in some way connected to potential energy (universal chemical potential). By providing a gradient of structural projections exhibiting gradual change of the average radius of the solid phase, or gradual change of surface wettability, or gradual change in surface tension due to spatial change of interface temperature or concentration of surfactant chemical, it is possible to provide a controllable and variable interface pressure gradient that could be used to manipulate fluid flow and separately regulate the flow of each fluid phase present in the bio-lamina bioreactor. This pressure gradient can contribute (with other pertinent forces like gravity, buoyancy, viscous, pressure, and inertial forces) to the discerning motion of the gas and liquid phases. In particular disclosed embodiments, the pressure gradient can be modified to improve solubility of particular gases flowed through the bio-lamina bioreactor. In particular disclosed embodiments, increasing the pressure gradient can increase the solubility of certain gases (e.g., methane) in other fluids (e.g., water) flowing through the device.

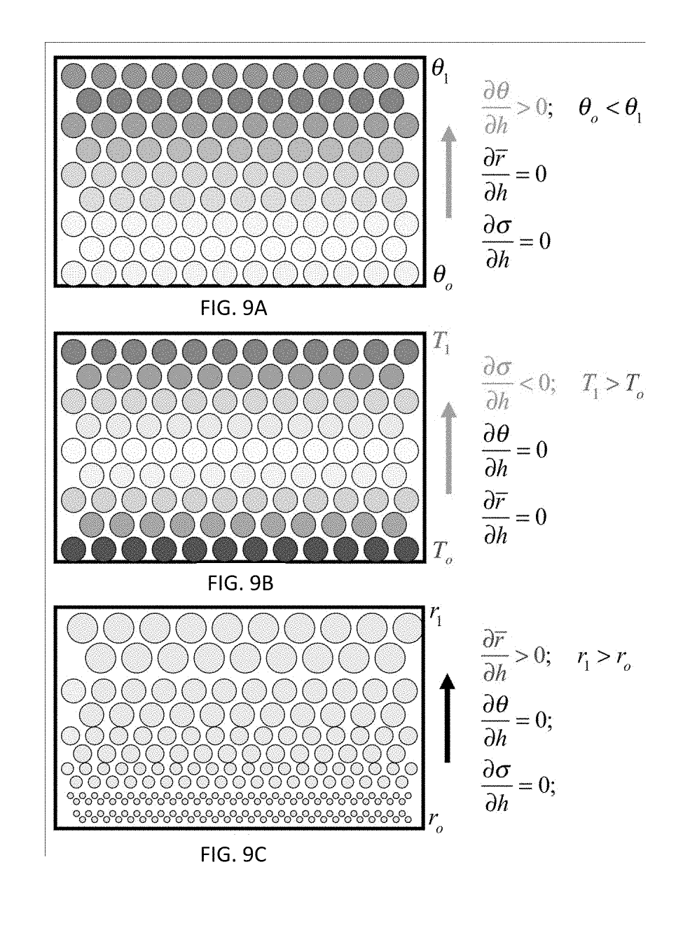

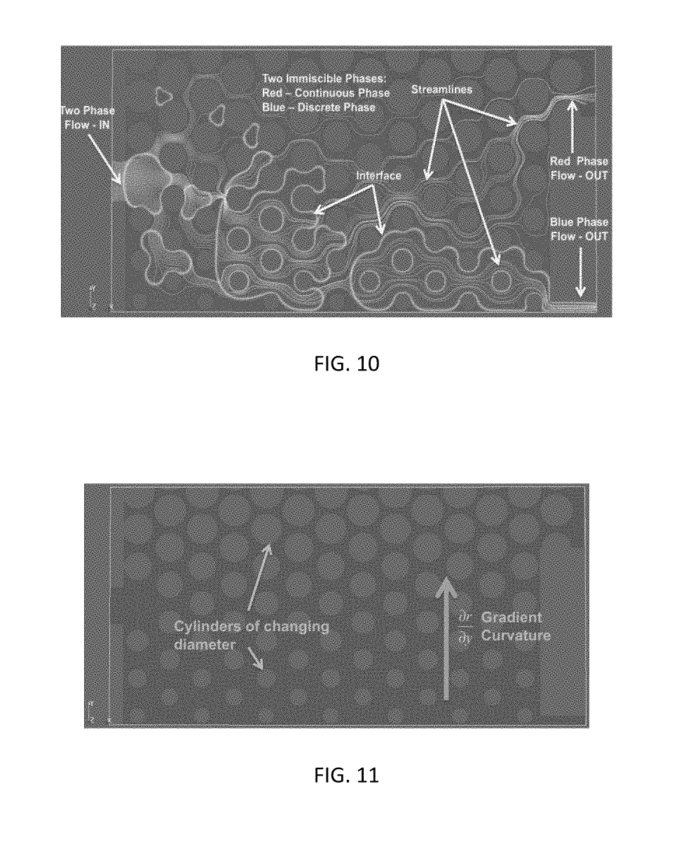

FIGS. 9A-9C provide schematic representations of exemplary possible gradient changes of properties, such as changes in contact angle ⊖ (colors represent different level of hydrophobicity of surfaces, FIG. 9A); changes in surface tension σ (difference in temperature—Marangoni effect, FIG. 9B); and changes in local curvature 1/r̅ (FIG. 9C). All gradients in properties may cause changes in pressure gradient. The circles illustrated in each of FIGS. 9A-9C represent the structural projections as described herein. FIG. 10 illustrates an exemplary numerical simulation of a two-phase flow through an engineered bio-lamina substrate comprising a plurality of gradient structural projections (FIG. 11). Concurrently with the depicted size gradients, the surface of the structural projections can further be modified so as to be enhanced with gradient coatings that change contact angle, or they can be exposed to local change in temperature or surfactant concentration, thus creating local surface tension gradients.

Each of the biofilm bio-lamina and fluid flow bio-lamina substrates can have any of the following dimensions. Also, the biofilm bio-lamina and fluid flow bio-lamina can have any suitable shape, such as rectangular, square, circular, and the like. In some embodiments, the bio-lamina substrates can have lengths ranging from greater than 0 mm to 10 m, or from 0.01 m to 10 m, such as 0.01 mm to 1 m, or 0.01 m to 0.1 m. In some embodiments, the length of a bio-lamina substrate was greater than 30 cm, such as greater than 34 cm. In some embodiments, the bio-lamina substrates can have widths ranging from greater than 0 mm to 10 m, or from 0.01 m to 10 m, such as 0.01 mm to 1 m, or 0.01 m to 0.1 m. In some embodiments, the width was greater than 20 cm. In some embodiments, the bio-lamina substrates can have thicknesses ranging from 100 μm to 100 cm, 300 μm to 900 μm, such as 400 μm to 800 μm, or 500 μm to 700 μm.

The bio-lamina substrates can be made of any material suitable for use with the fluids described herein. Suitable bio-lamina substrate materials include, without limitation, polymers, metals, ceramics, and cellulosic materials (e.g., cellulosic paper). Examples of suitable polymeric materials include polycarbonate, polyethylene terephthalate (PET), polyether imide (PEI), poly(methyl methacrylate) (PMMA), halogenated polyethylene, such as poly(tetrafluoroethylene) (PTFE), or combinations thereof. Metal bio-lamina substrates may be any that can have desired features formed therein, such as materials that can be photo-chemically etched or otherwise machined to have desired features, including blind features. Examples include stainless steels, copper, titanium, nickel, and aluminum, or combinations thereof. Other suitable bio-lamina substrate materials include, but are not limited to, metal oxides (e.g., silica, various glasses, ceramic materials, or the like), or films thereof supported on metal, polymer, ceramic, or cellulosic substrates. Ceramics may be selected from alumina, fused silica, quartz, and forms of glass and silicon wafers.

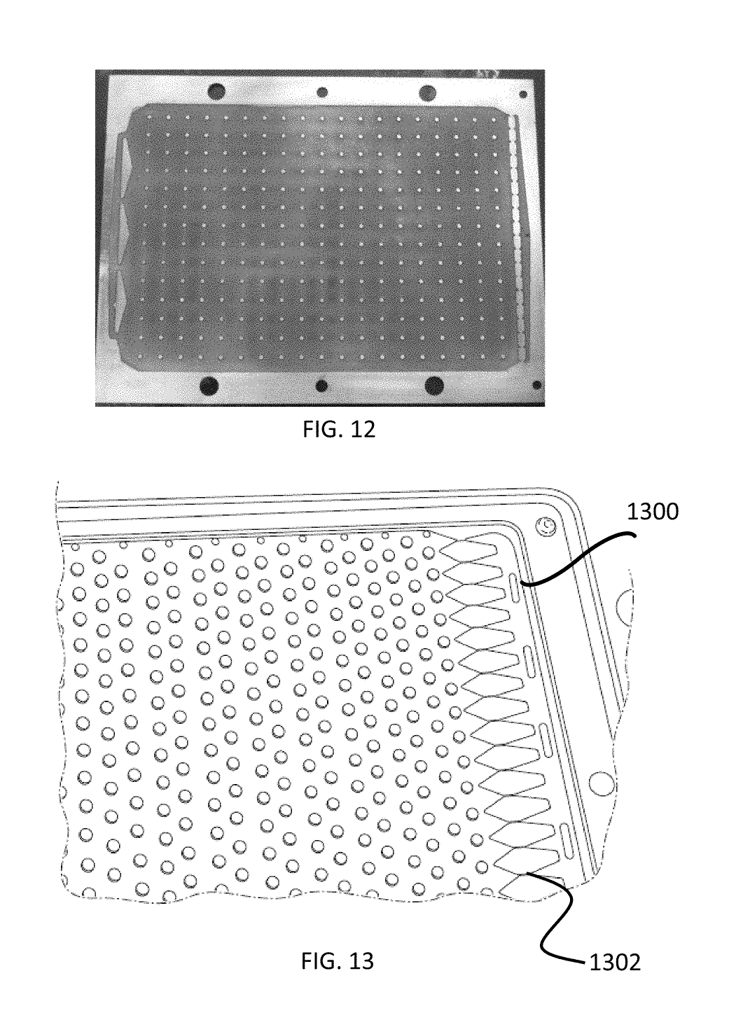

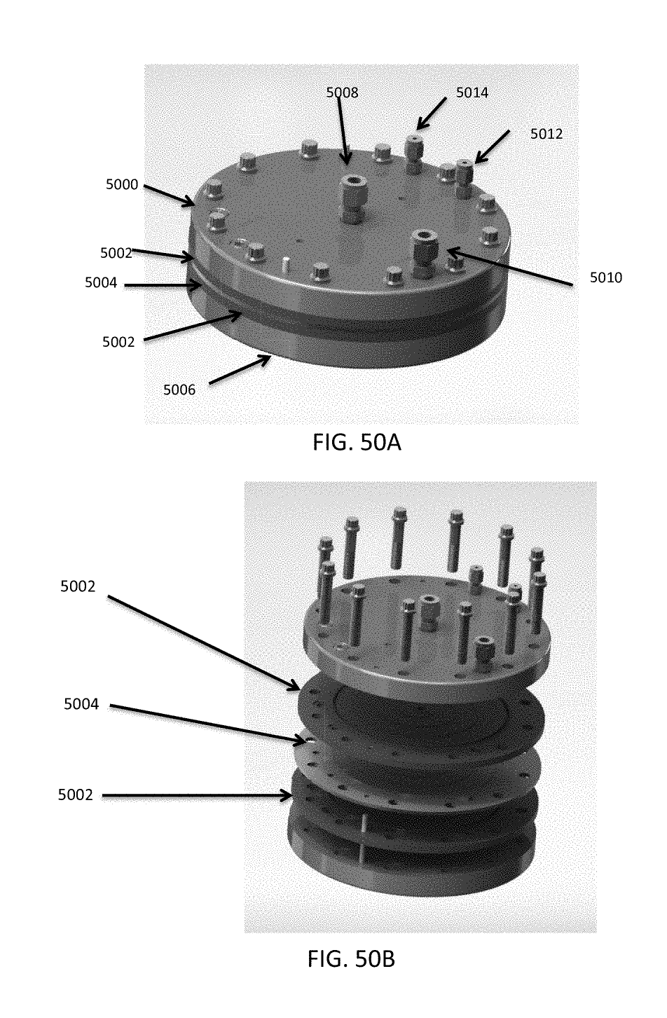

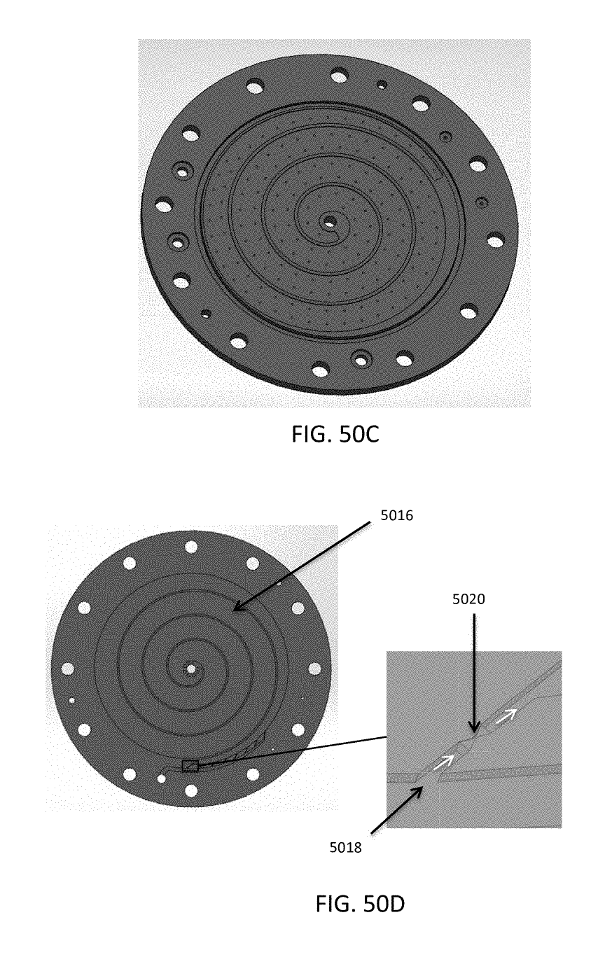

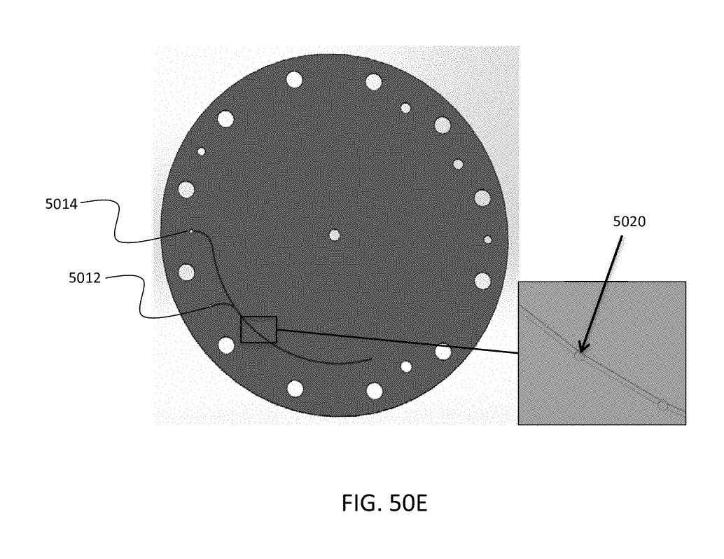

Embodiments of the biofilm bio-lamina substrate are coupled to a biofilm. In some embodiments, the biofilm bio-lamina substrate can be coupled to the biofilm by directly synthesizing the biofilm on the bio-lamina substrate. In other embodiments, the biofilm bio-lamina substrate can be coupled to the biofilm by first producing the biofilm and then coupling it to the first lamina substrate, such as with an adhesive. Methods for coupling the biofilm to the bio-lamina substrate directly are described above and illustrated in FIGS. 2-6. A photographic image of a bio-lamina substrate coupled to a biofilm is provided by FIG. 12. Another exemplary biofilm bio-lamina substrate is illustrated in FIG. 50C, which shows a disc-shaped biofilm bio-lamina substrate wherein each side of the disc comprises a plurality of structural projections as well as a spiral configured flow path. In some embodiments, the biofilm can be formed as a thin film on the biofilm bio-lamina substrate. Thin films include films having thicknesses ranging from 10 μm to 1 mm, such as 50 μm to 1 mm, or 75 μm to 1 mm.

The bio-lamina bioreactors further comprise a fluid flow bio-lamina substrate. The fluid flow bio-lamina substrate can be used to facilitate fluid flow through the bio-lamina bioreactor. The fluid flow bio-lamina substrate can comprise a plurality of structural projections similar to those of the biofilm bio-lamina substrate, but further comprise one or more channel manifolds. The channel manifolds comprises one or more openings through which fluids can be introduced. The channel manifolds can include a first channel manifold comprising a fluidic channel and an opening through which a liquid can be introduced. The channel manifolds also can include a second channel manifold comprising one or more fluidic channels and one or more openings through which one or more gases can be introduced. The fluidic channels of the first and second channel manifolds can have any of the following dimensions: a length ranging from 10 μm to 1 m, such as 100 μm to 0.1 m, or 100 μm to 0.01 m, a width ranging from 5 μm to 0.01 m, such as μm to 1000 μm, or 5 μm to 100 μm, and a depth ranging from 5 μm to 1000 μm, such as 5 μm to 500 μm, or 5 μm to 20 μm. In some embodiments, the first and second channel manifolds can have the same or different dimensions. In some embodiments, the first and second channel manifolds can be oriented parallel to one another, but other configurations are contemplated herein.

While some embodiments of the fluid flow bio-lamina substrates comprise separate openings for fluid introduction (e.g., separate openings for different gases), the fluid flow bio-lamina substrate also can be configured to comprise a single opening to allow introduction of a mixed gas system. In some embodiments, the openings can have the same or different diameters. The openings of the channel manifolds can have diameters ranging from 1 μm to 500 μm, such as 1 μm to 100 μm, or 1 μm to 5 μm.

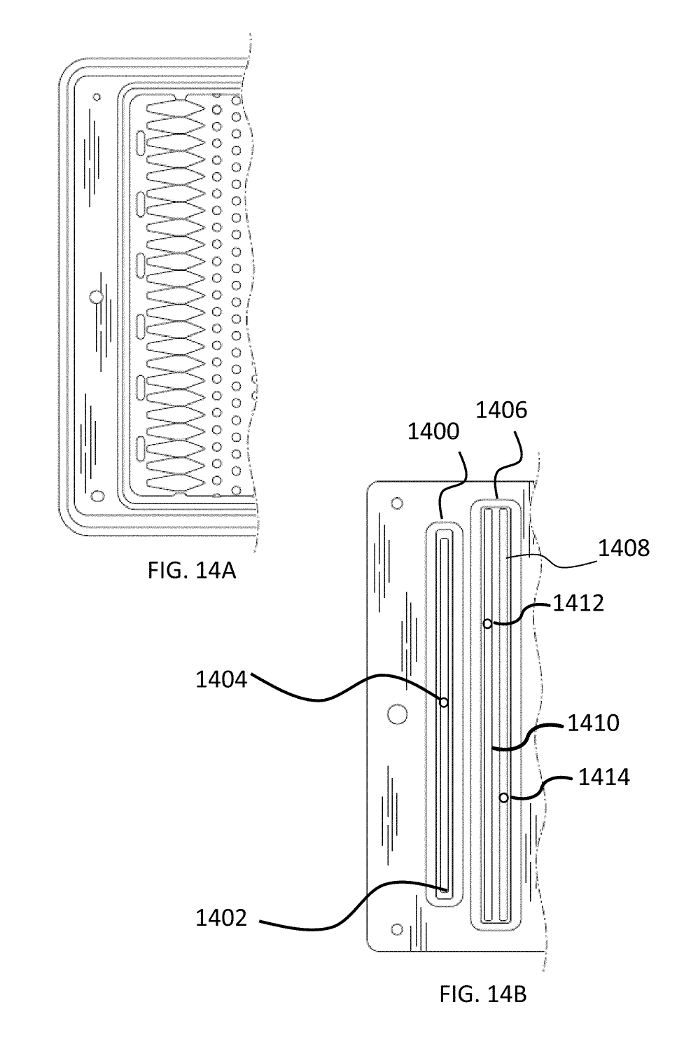

An exemplary channel manifold configuration is illustrated in FIG. 13. FIG. 13 provides a top plan (partial) view of a plurality of channel manifolds 1300 arranged near fluid mixers 1302. FIG. 14A provides another view of a channel manifold configuration and FIG. 14B provides an expanded top plan view of a single channel manifold configuration comprising two channel manifolds. As illustrated in FIG. 14B, a first channel manifold 1400 comprises a channel 1402 and an opening 1404 through which liquid can be introduced. FIG. 14B further illustrates a second channel manifold 1406 comprising two channels 1408 and 1410 and two openings 1412 and 1414 through which gases can be introduced into the bio-lamina bioreactor. Though the embodiment illustrated in FIGS. 14A and 14B positions the second channel manifold 1406 closest to the fluid mixers 1416 (FIG. 14A), other embodiments can switch the position of the first and second channel manifolds so that the first channel manifold 1400 is positioned closest to the fluid mixers 146.

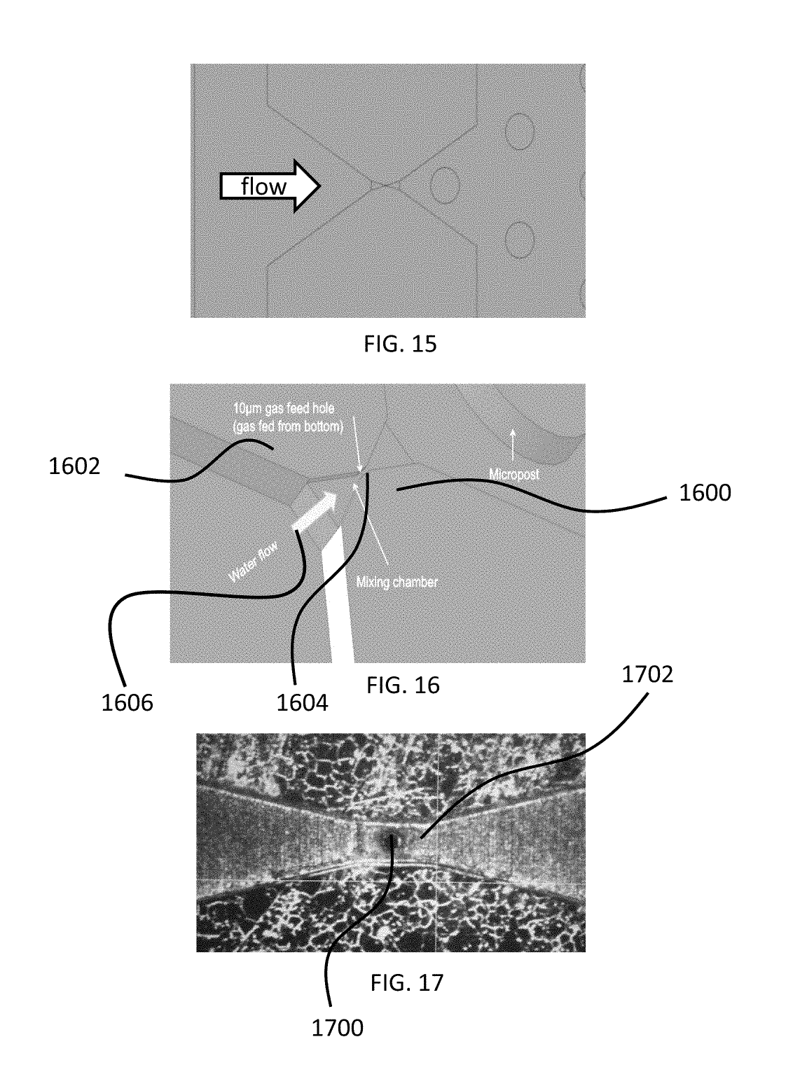

The fluid flow bio-lamina substrate can further comprise a plurality of fluid mixers. The fluid mixers can comprise a plurality of shaped protrusions. In some embodiments, different shapes can be used to provide fluid mixers having a tapered flow channel through which the liquid can flow and mix with gas that is introduced into the flow channel. The fluid mixers can be laser micro-machined into the fluid flow bio-lamina substrate in a location near the channel manifolds such that when liquid enters through an opening of the channel manifolds it flows towards the fluid mixers and passes through a tapered flow channel of the fluid mixers. The fluid mixers further comprise a feed hole within the tapered flow channel through which gas (or gases) can be fed. The fluid mixers provide fast flowing liquid to break-up gas flow to form bubbles. An exemplary fluid mixer comprising a tapered flow channel is illustrated in FIGS. 15 and 16. FIG. 15 provides a top plan view of a fluid mixer and FIG. 17 is an image showing an expanded view of an exemplary fluid mixer. FIG. 16 illustrates the features of an exemplary fluid mixer. According to the embodiment illustrated in FIG. 16, the fluid mixer comprises elevated projections 1600 and 1602 that are configured to provide a tapered flow channel 1604 through which liquid can flow (indicated as arrow 1606) so as to be mixed with gas provided by feed hole (not illustrated). The tapered flow channel can have a length ranging from 100 μm to 0.05 m, such as 100 μm to 5000 μm, or 100 μm to 1000 μm. As illustrated in FIG. 17, feed hole 1700 is positioned so as to be located in the flow path of the liquid to facilitate mixing between the liquid and the organic species introduced into the device through the feed hole. Another exemplary embodiment of a fluid flow bio-lamina substrate is illustrated in FIGS. 50D and 50E. The embodiment illustrated in FIGS. 50D and 50E illustrate a disc-shaped (or circular) fluid flow bio-lamina substrate comprising flow channels 5016 in a spiral configuration and a fluid mixer component 5018 (illustrated in the zoomed portion of FIG. 50D), which comprises feed hole 5020. The opposite side of the fluid flow bio-lamina substrate of FIG. 50D is illustrated in FIG. 50E. As illustrated in FIG. 50E, the opposite side of the fluid flow bio-lamina substrate comprises ports 5012 and 5014 for introducing the gases that are added into the device. The zoomed portion of FIG. 50E further illustrates feed hole 5020 and its location within the channel through which the gases flow.

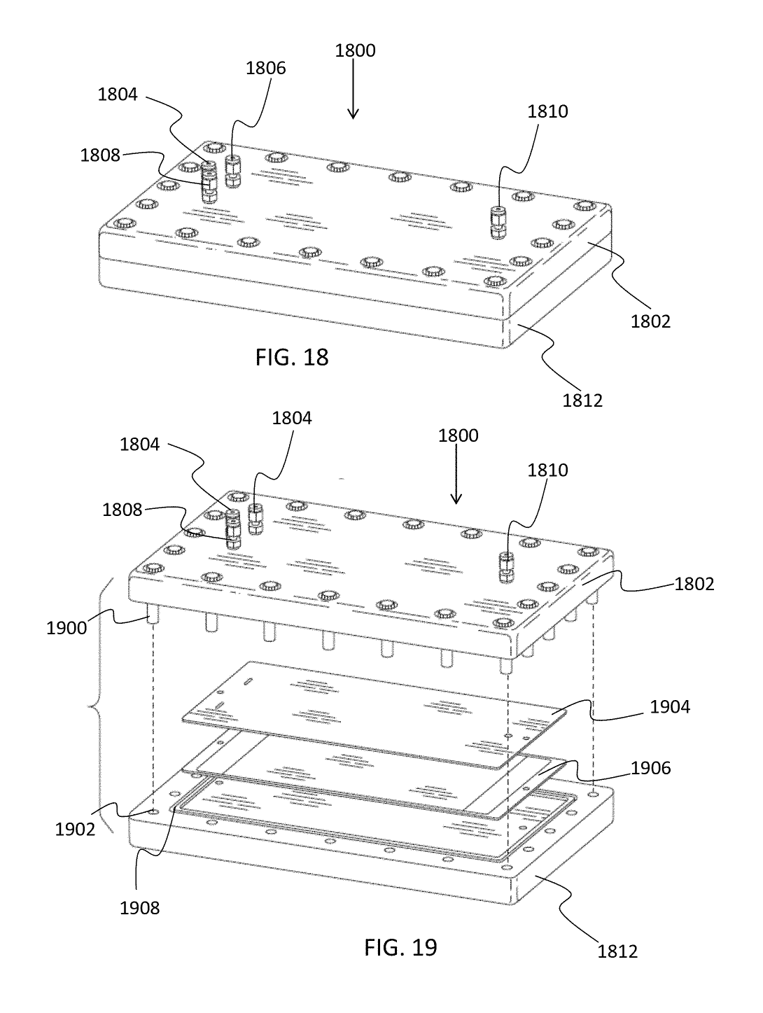

The bio-lamina bioreactors disclosed herein can further comprise top and bottom clamp plates configured to maintain the biofilm bio-lamina and fluid flow bio-lamina substrates in a desired position and orientation. The top clamp plate can comprise one or more coupled ports that facilitate fluid delivery into the bio-lamina bioreactor. In some embodiments, the top clamp plate can be coupled to at least one port configured to deliver liquid into the bio-lamina bioreactor, one or more ports configured to deliver gases into the bio-lamina bioreactor, and at least one outlet port configured to deliver fluids from the bio-lamina bioreactor. In some embodiments, the top clamp plate can comprise one or more alignment pins that can extend into one or more alignment holes of the bottom press plate. The top and bottom clamp plates can comprise metal, with exemplary embodiments comprising stainless steel, or aluminum. FIGS. 18 and 19 illustrate exemplary clamp plates and further illustrate how the clamp plates and bio-lamina substrates are configured together in a bio-lamina bioreactor. FIG. 18 illustrates an exemplary bio-lamina bioreactor 1800 comprising a top clamp plate 1802 comprising three different fluid inlets 1804, 1806, and 1808, and a fluid outlet 1810, and bottom clamp plate 1812. FIG. 19 illustrates an exploded perspective view of a bio-lamina bioreactor 1800. Device 1800 comprises top clamp plate 1802 comprising a plurality of alignment pins 1900 and inlets 1804, 1806, and 1810 and fluid outlet 1810. Bottom clamp plate 1812 comprises a plurality of alignment holes 1902 positioned to accept the alignment pins of top clamp plate 1802. A fluid flow bio-lamina substrate 1904 is positioned adjacent to the top clamp plate 1802 and a biofilm bio-lamina substrate 1906 is positioned adjacent to bottom clamp plate 1812. Another exemplary set-up of a bio-lamina bioreactor is illustrated in FIGS. 50A and 50B. According to the embodiment in FIG. 50A, the bio-lamina bioreactor can have a disc shape and can comprise a top clamp plate 5000, a bottom clamp plate 5006, two biofilm bio-lamina substrates 5002, and fluid flow bio-lamina substrates 5004. The embodiment illustrated in FIG. 50A further comprises fluid inlet 5010 and fluid outlet 5008. Gas inlets 5012 and 5014 also are provided. FIG. 50B illustrates an exploded perspective view of the embodiment illustrated in FIG. 50A.

In yet additional embodiments, one or more O-ring seals can be used to hermetically seal the top clamp plate and the bottom clamp plate so as to prevent fluids from leaking from the bio-lamina bioreactor. The O-ring seals can be joined with the bottom or top clamp plate by placing the O-ring seals into grooves formed within the bottom or top clamp plate. For example, device 1800 illustrated in FIG. 19 includes bottom clamp plate 1812 that comprises a groove 1908, which can accept an O-ring seal to hermetically seal bio-lamina bioreactor 1800. One or more fasteners also can be used to further secure and seal the components together.

The bio-lamina bioreactor is used in combination with additional components during operation. In some embodiments, the bio-lamina bioreactor is used in combination with one or more of the following components: a pump, one or more fluid pressure gauges, valving, a regulator, tubing, thermocouples, thermometers, and/or heat exchangers. The pressure gauges can include a liquid pressure gauge, a liquid inlet pressure gauge, a liquid outlet pressure gauge, a gas pressure gauge, and a rector outlet gauge. Suitable valving can comprise solenoid valves and/or three-way valves used to control gas flow into the bio-lamina bioreactor. One or more outlet tubes can be coupled to the fluid outlet so as to deliver fluid from the bio-lamina bioreactor, and the thermocouples and thermometers can be used to measure the temperature of the bio-lamina bioreactor.

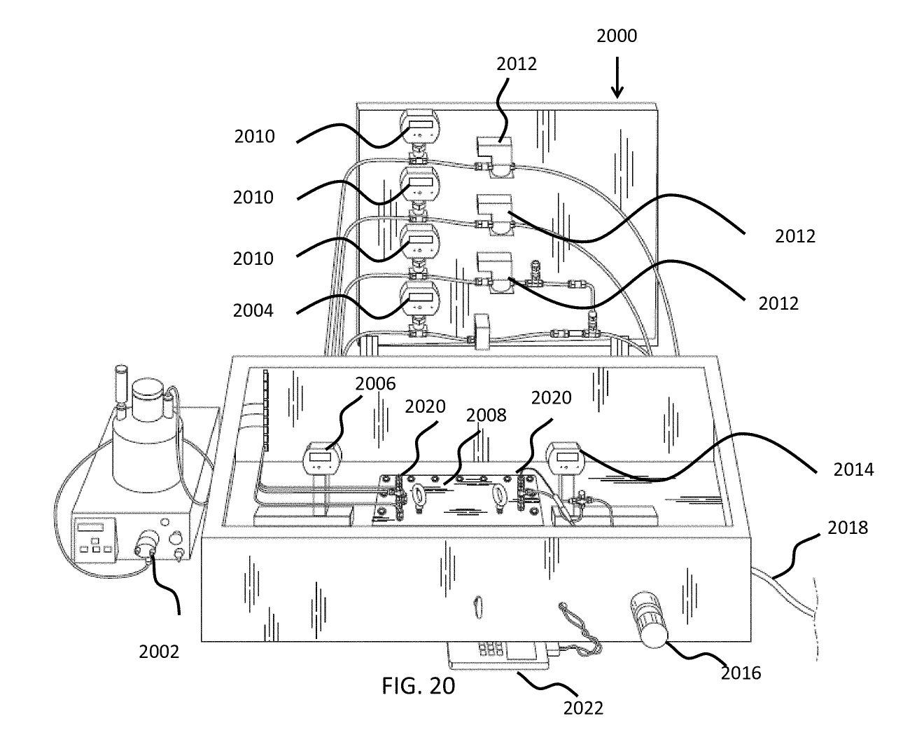

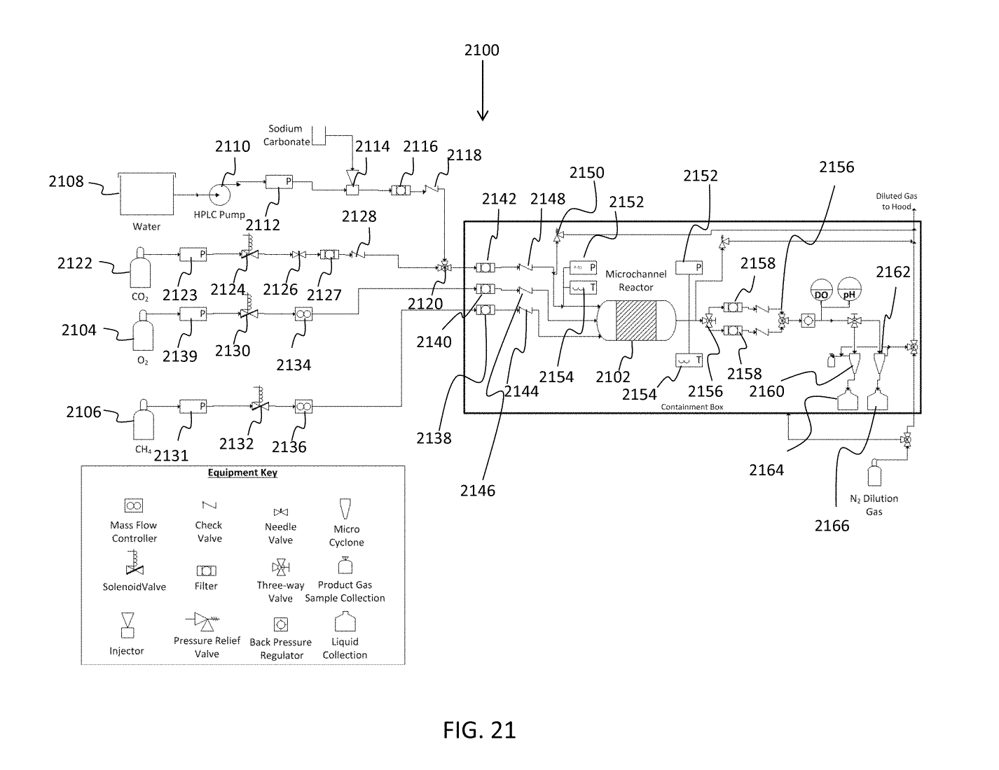

FIG. 20 illustrates an exemplary bio-lamina bioreactor set-up 2000 comprising a bio-lamina bioreactor embodiment and additional components for use. The embodiment in FIG. 20 illustrates a device set-up 2000 comprising a pump 2002 for introducing a liquid into the bio-lamina bioreactor, which is connected to a liquid pressure gauge 2004. A liquid inlet pressure gauge 2006 also is coupled to bio-lamina bioreactor 2008 to measure and control the pressure at which the liquid is introduced into the bio-lamina bioreactor. Gas pressure gauges 2010 also can be used and coupled to solenoid flow valves 2012 that are used to control introduction of gas (or gases) into the bio-lamina bioreactor through tubes used to flow gas to one or more gas inlets. A reactor outlet pressure gauge 2014 can be used to measure and control the pressure of the fluid exiting the bio-lamina bioreactor 2008 and a backflow pressure regulator 2016 also can be used to prevent backflow of the fluid passing through the bio-lamina bioreactor. Fluid exiting the device is passed through tube 2018 and ultimately collected in an external reservoir. The temperature of the bio-lamina bioreactor can be monitored using one or more thermocouples 2020 and a thermometer 2022.

FIG. 21 is a schematic diagram illustrating the connections and configuration of a bio-lamina bioreactor set up (device 2100). As illustrated in FIG. 21, bio-lamina bioreactor 2102 is coupled to gas sources 2104 and 2106, as well as water source 2108. Pump 2110 can be used to deliver water from water source 2108 and the delivery pressure can be monitored with pressure gauge 2112. An injector 2114 can be used to introduce sodium carbonate into the water flow to adjust pH, which passes through filter 2116 and check valve 2118. A three-way valve 2120 can be used to control water flow, and in some embodiments carbon dioxide flow (delivered via CO₂ tank 2122 and pump 2123, which connects to three-way valve 2120 via solenoid valve 2124, needle valve 2126, filter 2127, and check valve 2128). The different gases used in bio-lamina bioreactor 2102 can be introduced from gas sources 2104 and 2106 and the flow of the gases can be introduced using pumps 2129 and 2131 and controlled using solenoid valves 2130 and 2132 and mass flow controllers 2134 and 2136. Additional filters (2138, 2140, and 2142) and check valves (2144, 2146, and 2148) can be used to further control purity and flow of the fluids into bio-lamina bioreactor 2102. A pressure relief valve 2150 can be used to reduce pressure build up as needed when fluids enter bio-lamina bioreactor 2102. Pressure gauges 2152 and thermocouples 2154 can be used to further monitor pressure and temperature of bio-lamina bioreactor 2102 during operation. As fluids exit bio-lamina bioreactor 2102, the flow of the fluids can be controlled using three-way valves 2156 and additional filters 2158 can be used to control purity of the fluids exiting the device. Fuel collection can be facilitated using a combination of microcyclones 2160 and 2162 and collection vessels 2164 and 2166.

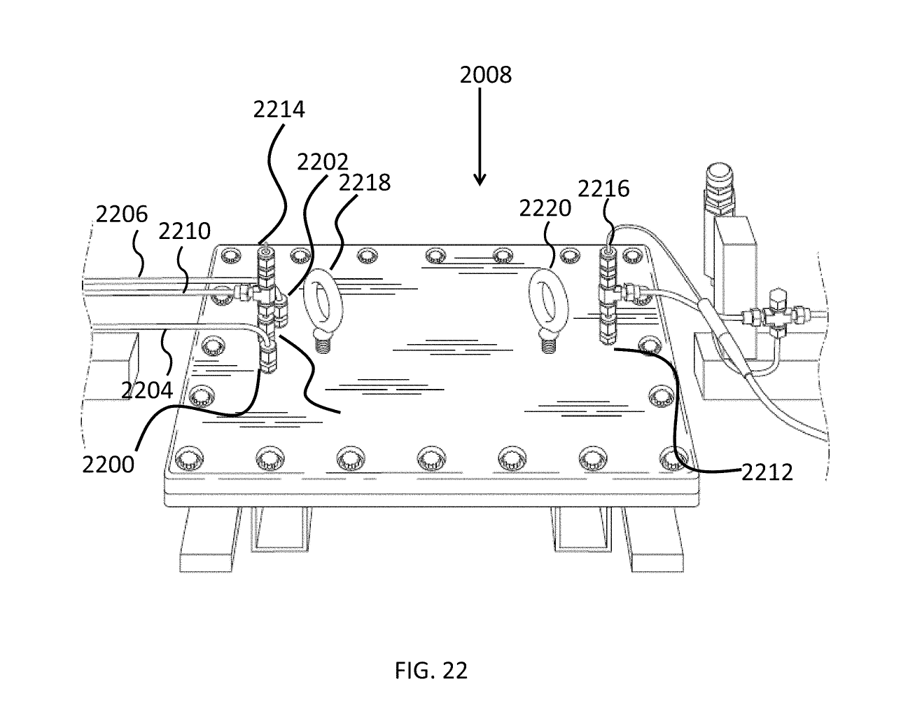

FIG. 22 provides another view of bio-lamina bioreactor 2008. In the embodiment illustrated in FIG. 22, gas inlets 2200 and 2202 can be connected to gas sources through tubes 2204 and 2206 respectively. While two gas inlets are illustrated in FIG. 22, one gas inlet also can be used to introduce into the bio-lamina bioreactor a mixed gas system. With reference to FIG. 22, water can be introduced into bio-lamina bioreactor 2008 through water inlet 2208 and tube 2210. After the water and gas pass through bio-lamina bioreactor 2008 and interact with the biofilm contained therein, the resulting products can be delivered from the bio-lamina bioreactor via outlet 2212. The water inlet 2208 and outlet 2212 can further be coupled to thermocouples 2214 and 2216, respectively. Fasteners 2218 and 2220 can be used to further secure and seal bio-lamina bioreactor 2008.

### IV. Methods of Making Bio-Lamina Bioreactors

The bio-lamina bioreactors disclosed herein can be made using methods described below. Methods for making particular components of the bio-lamina bioreactors also are disclosed.

In some embodiments, the bio-lamina bioreactors can be made by coupling the components described above so as to provide bio-lamina bioreactors capable of continuous operation at elevated pressure conditions to produce a variety of products from the reactants introduced into the bio-lamina bioreactors. In particular disclosed embodiments, a bottom clamp plate is coupled to the biofilm bio-lamina substrate so that the bottom surface of the bio-lamina substrate contacts the top surface of the bottom clamp plate and the biofilm coupled to the bio-lamina substrate is positioned to face the fluid flow bio-lamina substrate, which is associated with a top clamp plate. The fluid flow bio-lamina substrate can be physically associated with the biofilm bio-lamina substrate so that the structural projections of each bio-lamina substrate are in alignment with one another. The top and bottom clamp plates are configured to ensure that the biofilm bio-lamina substrate and the fluid flow bio-lamina substrate remain aligned when the device is constructed and during use.

In some embodiments, a single bio-lamina bioreactor is used. In other disclosed embodiments, a plurality of bio-lamina bioreactors can be used. In such embodiments, the plurality of bio-lamina bioreactors can be coupled together linearly, in parallel, and or in series. In embodiments where a plurality of bio-lamina bioreactors is coupled in parallel, the bio-lamina bioreactors can be stacked on top of one another so as to build upwards in a parallel fashion. A stack of bio-lamina substrates can be clamped with a clamping device, which can comprise the top and bottom clamp plates described herein. FIGS. 50A and 50B illustrate an exemplary embodiment wherein a plurality of bio-lamina substrates can be used in combination with one or more fluid flow bio-lamina substrates. In embodiments where a plurality of bio-lamina bioreactors is used, one or more heat exchangers can be used to absorb heat produced by the plurality of bioreactors so as to preserve operation of the bio-lamina bioreactors. Suitable heat exchanges are recognized by those of ordinary skill in the art and in some embodiments, the heat exchanger can simply comprise a flow of cold water that is passed over a top or bottom surface of the bio-lamina bioreactor. In some embodiments, one heat exchanger can be positioned between every set of five or more bio-lamina bioreactors.

In some embodiments, the strength of the biofilm produced using the above internal gelation system, or the other described methods contemplated by the present disclosure, can be determined using planned shear mechanical testing of the film-forming materials. In some embodiments, the films of the material are adhered between two shims and the shims are forced to slide apart with a mechanical testing machine. This test can accurately measure the shear forces that the film-forming materials can withstand. A compression test may also be done with the mechanical testing machine for further determination of the film-forming material's internal cohesion properties.

### V. Methods of Using Bio-Lamina Bioreactors

Disclosed herein are methods of using the disclosed bio-lamina bioreactors. The disclosed bio-lamina bioreactors can be used to produce a variety of fuels from organic reactants. The bio-lamina bioreactors disclosed herein comprise biofilms containing one or more microorganisms that are able to convert the organic reactants to metabolic products or fuels. The disclosed bio-lamina bioreactors can produce fuels at levels that are not feasible using conventional bioreactors, such as chemostats.

In some exemplary embodiments, the disclosed bio-lamina bioreactors can be used to make fuels (e.g., methanol) from organic precursors (e.g., methane and oxygen). In yet other embodiments, the disclosed bio-lamina bioreactors can be used to make other products, such as multi-carbon alcohols (e.g., ethanol, butanol) or other oxidized organic species (e.g., formaldehyde or acetaldehyde, formic or acetic acid). In addition, a variety of other microorganism(s) and/or immobilized enzyme(s) could be used to produce a number of chiral, achiral or racemic products, including fine chemicals and pharmaceutical precursors.

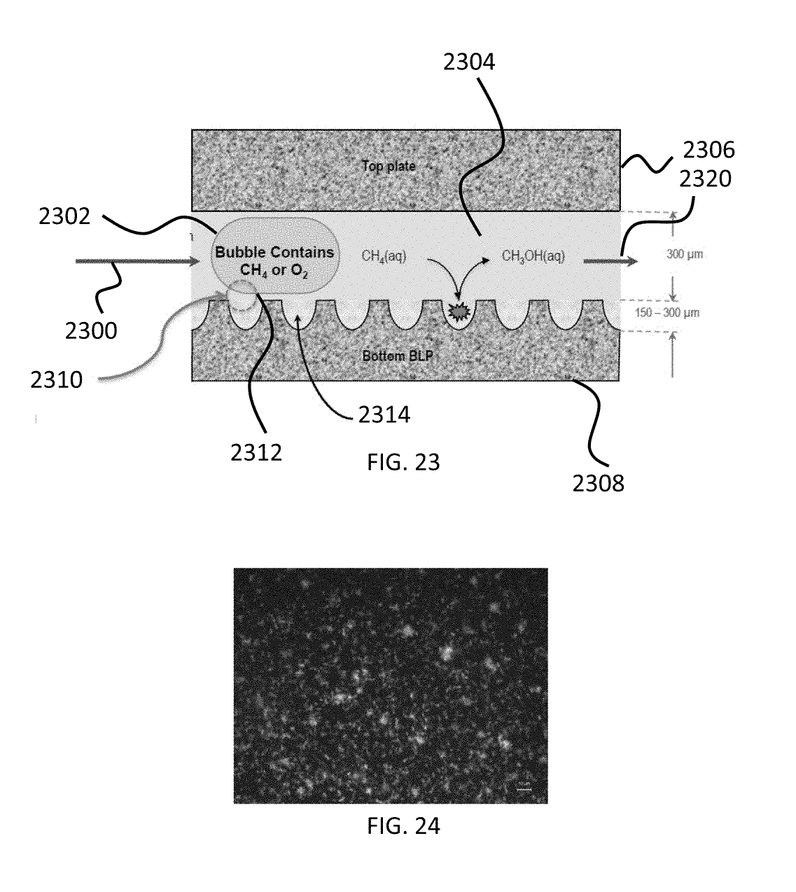

During operation of the bio-lamina bioreactor, one or more gases are allowed to flow through the microchannels of the bio-lamina plates. The flow of gas bubbles is facilitated by the liquid that also is introduced into the device. As the liquid and gas bubbles flow through the microchannels of the bio-lamina bioreactor, a gas/liquid mass transfer area develops on the outer perimeter of the bubbles, which is able to contact the biofilm of the biofilm bio-lamina as the bubbles pass through the device. Thus, a gas-to-liquid-to-biofilm transport mechanism is enabled, where the transport/interaction between the gas bubbles and the biofilm occurs at the interface between a gas bubble and the surface of the biofilm. The surface of the biofilm typically is hydrophilic and a thin interface layer of water forms between the bubble and the biofilm. This interface can be saturated with one or more of the gases at equilibrium conditions such that the concentration of the one or more gases (e.g., methane) is highest at this interface. This increased gas concentration then facilitates a more efficient conversion of the gas to the desired fuel after interacting with the microorganism cells present in the biofilm, without the long diffusion times required by conventional reactors. FIG. 23 provides a schematic illustration of the interactions that take place as the gas bubbles are delivered through flow paths of a bio-lamina bioreactor. As illustrated in FIG. 23, fluid flow (indicated as arrow 2300) carries a gas bubble 2302 through a microchannel 2304 formed between the fluid-flow bio-lamina 2306 and the biofilm bio-lamina 2308. As gas bubble 2302 flows through microchannel 2304, a gas/liquid mass transfer area 2310 is formed between the bubble interface 2312 and biofilm layer 2314, which is positioned within spaces defined by structural projections 2316. The microorganism cells (e.g., 2318) contained within the biofilm layer 2314 are then able to interact with an increased concentration of gases present at the bubble interface 2312 to convert the gas to a fuel which then continues through microchannel 2304 (indicated as arrow 2320).

In some embodiments, bio-lamina bioreactor operation can include utilizing a liquid flow of up to 500 mL/hr (12 L/day) and gas flow of up to 5,000 mL/hr total (at 1 atm). The bio-lamina bioreactor can be operated at an operating pressure ranging from greater than 0 bars to 50 bars, such as 1 bar to 50 bar, 1 bar to 20 bar, or 1 bar to 5 bar. In some other embodiments, the bioreactor can be operated at operating pressures ranging from 10 bar to 50 bar, or 20 bars to 50 bars, or 30 bars to 50 bars. In some embodiments, the operating pressure can be modified (increased or decreased) to influence the aqueous solubility of the gases used. Solely by way of example, an operating pressure of 20-30 bars was used for embodiments using methane and oxygen to improve the stability and solubility of these gases in water used to facilitate flow through the fluid flow bio-lamina.

In some embodiments, a mixture of gases is used. Suitable gases can be introduced into the reactor, to serve either as a reactant or carrier. In some embodiments, chemical compatibility will determine the type of gas used in the bioreactor. In some embodiments, the gases can be selected from sparingly-soluble gases (e.g., hydrogen, methane, hydrocarbons, carbon dioxide, oxygen, or mixtures thereof). Solely by way of example, oxygen can be used in an amount ranging from 5% to 100% v/v, methane can be used in an amount ranging from 0% to 80% v/v, nitrogen can be used in an amount ranging from 0% to 20% v/v, and carbon dioxide can be used in an amount ranging from 0% to 5% v/v. In exemplary embodiments, the mixed gas system can comprise a ratio of a source gas (e.g., methane) to oxygen. In such embodiments, the ratio of source gas to oxygen can range from 95:5 v/v to 0:100 v/v, such as 25:75 v/v to 75:25 v/v, or 33:67 v/v to 50:50 v/v. In exemplary embodiments, a mixed gas system comprising 1/3 methane gas (v/v) and 2/3 oxygen (v/v) was used.

### VI. Examples

### Example 1

In this example, a method for encapsulating OB3b cells in calcium alginate gels in stainless steel microreactors is described. Briefly, a 316L stainless steel (“SS”) surface is first cleaned by successive sonication in acetone for initial degreasing then deionized water (removal of salts), toluene (secondary degreasing), acetone (remove toluene film), and DI water (remove residual solvent). The SS surface is then passivated in 32.5% nitric acid. A solution of sodium alginate (2 wt %, Sigma-Aldrich 71238) with suspended cells is prepared from equal parts of washed and concentrated OB3b cell slurry with a 4 wt % sodium alginate solution in water. The alginate/cell suspension is spread evenly on the passivated SS, and briefly degassed under low vacuum to eliminate bubbles. The fluid alginate layer is sprayed with 0.5M CaCl₂ to crosslink and stabilizes the surface, and then immersed in 0.05M CaCl₂ solution to complete the calcium ion-induced gelation. The initial spray pre-treatment was used to prevent the alginate slurry from being displaced by the inevitable fluid motion during immersion in the bulk CaCl₂ solution. Such methods reliably produce uniform, conformal and flat films containing only 1.5 wt % OB3b cells in a 2 wt % alginate matrix.

The method outlined above has been shown to produce stable, firm calcium alginate gels with or without suspended OB3b cells. On properly cleaned and passivated stainless steel, the alginate gel films adhere strongly enough to prevent their peeling or sloughing, and are expected even when exposed to fluid flow rates much greater than within the microreactor. The maximum operating shear rate (and hence fluid flow rate) within the reactor can be determined, as can the long-term stability of alginate containing immobilized cells and this information can be used for device modification if needed. A fluorescent dye-based live/dead stain protocol (BacLight™, Life Technologies) for use with cells immobilized in calcium alginate can be used by adding a “destain” step to remove excess stain from the gel matrix prior to imaging (FIG. 24). In some embodiments, live and dead controls were performed in PBS solution. In the live control, many cells fluoresced under a GFP filter and a few fluoresced under the Texas Red filter indicating that a vast majority of cells were alive. In the dead control cells were treated with isopropyl alcohol to kill them. A longpass filter was used which shows both green and red fluorescence. No green fluorescence was seen, while many cells fluoresced red indicating that all the cells were dead. A dead control was performed with cells in a thin alginate gel. Cells were killed with isopropyl alcohol prior to being added to alginate solution, which was then solidified with calcium chloride. The immobilized cells fluoresced both green and red indicating that the propidium iodide stain did not fully quench the Syto 9 stain. In exemplary embodiments, it was determined many cells survived the gel encapsulation process used in the embodiments. Live cells were added to liquid alginate which was then solidified by immersion in a CaCl₂ solution. Analysis of the photos shows that 93% of the cells survived the alginate immobilization process.

### Example 2

Cell-Immobilization in Cells Spherical Bead of Alginate:

The microbial conversion of methane to methanol by Methylosinus trichosporium (OB3b) was evaluated in an immobilized cell packed bed reactor (PBR). The approach here was to evaluate the potential for methanol formation in a reactor that was easier to perform that the Drip Flow reactors. OB3b cells were immobilized within spherical alginate hydrogel beads that served as packing for the PBR. An aqueous alginate solution of 2 wt % sodium alginate was pumped through a 23 gauge stainless steel needle under drop conditions given by Bond and Weber numbers and dropped into a 0.1 M CaCl₂ solution, which induced solidification of the beads by cross-linking of Ca²⁺ with the alginate polymer. Initial cell immobilization procedure began by centrifuging 40 mL of working free cell culture with an OB3b cell density of 0.389 g/L. Forty mL of 2.wt % alginate was added to the centrifuged culture and re-suspending the cells uniformly and maintaining a cell density of 1 g/L. The immobilization of living OB3b cells within alginate resulted in gel beads of roughly 2.5 mm in diameter.

Cell Activity in the Alginate Beads with pMMO-Expressing Ob3b:

Batch kinetic tests were performed with immobilized cell beads placed into 28 septum vial containers. The rates obtained with immobilized cells were compared with rates obtained with the same biomass of suspended cells. Each vial contained 10 mL beads, 8 mL growth media, and 9 mL headspace to which 0.2 mL of methane was added. The vials were rigorously shaken to achieve effective mass transfer. Headspaces were periodically analyzed for methane to estimate the rate of methane uptake via GC analysis.

Over the 3.5 hour batch test the average suspended cell conversion was 93±7.3% while average immobilized conversion was 77±7.3% Immobilized beads retained roughly 83% activity.

Batch Kinetic Experiments:

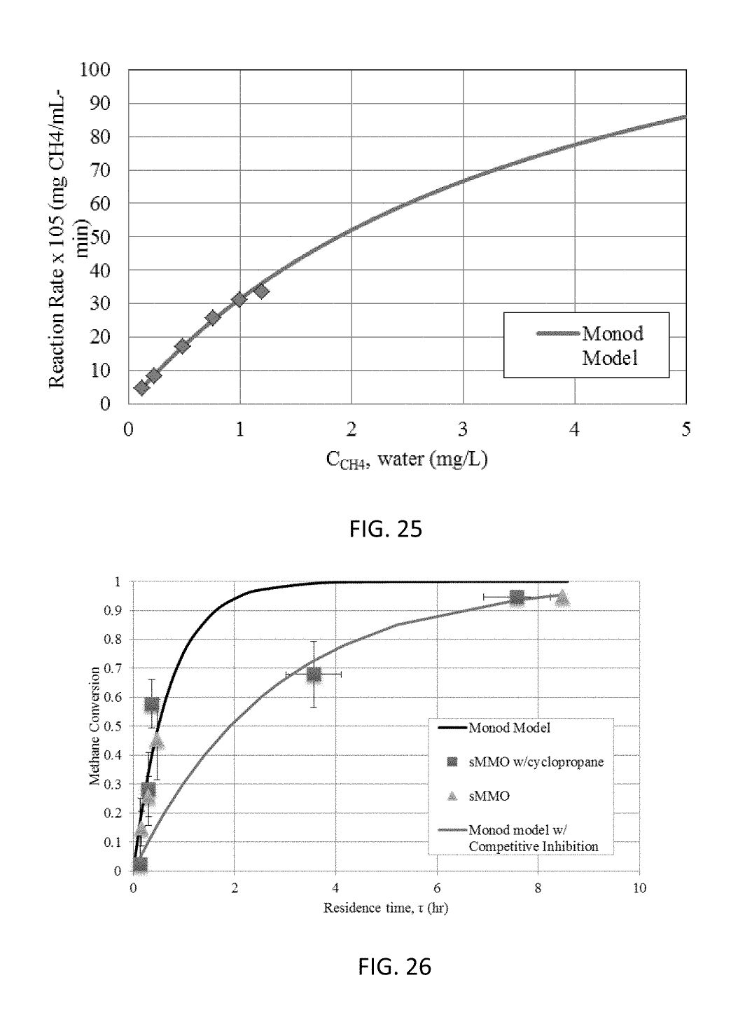

Kinetic batch experiments were performed to analyze the rate of methane consumption in the immobilized alginate matrix over a range of initial aqueous methane concentrations from 0.1 to 1.2 mg/L (FIG. 25). An increase in rate was observed with the increase in aqueous methane concentration that fit a standard Monod kinetics using the Lineweaver-Burk method of linearization (FIG. 26). The Monod model had an rₘₐₓ of 1.52×10⁻³ [mg CH₄/mL-min] and a Kₛ, of 3.84 [mg/L]. Rate units were on a void liquid volume basis so they can easily be used in the PFR reactor analysis. The rates also can be evaluated over a boarder range of concentrations.

Conversion to Methane to Methanol in a Packed Bed Reactor:

A Packed Bed Reactor was used to evaluate the conversion of methane to methanol by cell of OB3b expressing sMMO. Cyclopropanol interacts with MDH and irreversibly inhibits MDH activity via reaction shown in the equation below. Cyclopropanol is expected to be produced via oxidation of cyclopropane by MMO in this example.

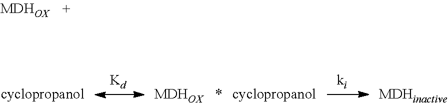

Cyclopropanol can be difficult to purchase and it is also unstable. Therefore, the production of cyclopropanol from the oxidation of cyclopropane by MMO was evaluated.

A continuous flow PBR was constructed using a glass HPLC column with the dimensions 2.5 cm ID and 13 cm packing height. Methane, oxygen, and the inhibitor feed solutions dissolved in media were contained within the two 100 mL syringes. The column was packed with alginate beads with sMMO-expressing OB3b formed in-situ under aseptic conditions Immobilized culture performance was evaluated in the PBR over a range of flow rates to achieve reactor residence times (τ) ranging from 0.2, 0.3, 0.5, and 8.5 hours. Two separate experiments were carried out, one for methane transformation (conversion) without the cyclopropane inhibitor and the other with the inhibitor (FIG. 26). For the methane utilization studies, the influent solution was developed by adding 7 mL methane and 21 mL oxygen to the 100 ml syringes. For the inhibition studies 1 ml of cyclopropane was added. After equilibration for 20 minutes, the gases were ejected from the syringes, and the syringes connected to the column inlet.

The fractional transformation of methane increased as the fluid residence time increased for both the uninhibited and inhibited cases (FIG. 26). About 50% and 90% of the methane was transformed (conversion) with a residence time of about 0.5 hours and 8 hours, respectively. Inhibition of methane transformation was observed in the cyclopropane-amended column indicating the interaction with sMMO.

An integrated form of the Monod equation was used to analyze the results from the PBR experiment. It relates substrate conversion across a packed bed reactor, X, to the residence time, τ (min), and initial substrate concentration Sₒ (mg/L).

$$τ = \frac{1}{η {r}_{max}} {XS}_{o} - \frac{{K}_{s}}{η {r}_{max}} ln ( 1 - X )$$

The solids lines in FIG. 26 were computed with the integrated Monod model using the Kₛ and kₘₐₓ obtained from the batch data. The simulation used an effectiveness factor (η) of 1 indicating no loss in cell activity. Good fit was obtained between the simple analytical solution, equation above, and the experimental observations for the uninhibited case. The maximum conversion was 95±0.8% at a τ of 8 hours. For the cyclopropane inhibition embodiment, the same kₘₐₓ as non-inhibited case was used, but with a Kₛ four times the uninhibited case to investigate the plausibility of competitive inhibition. The adjusted Monod kinetics provided a poorer fit for conversions at low residence times, but had a better at τ=4 and 8 hours.

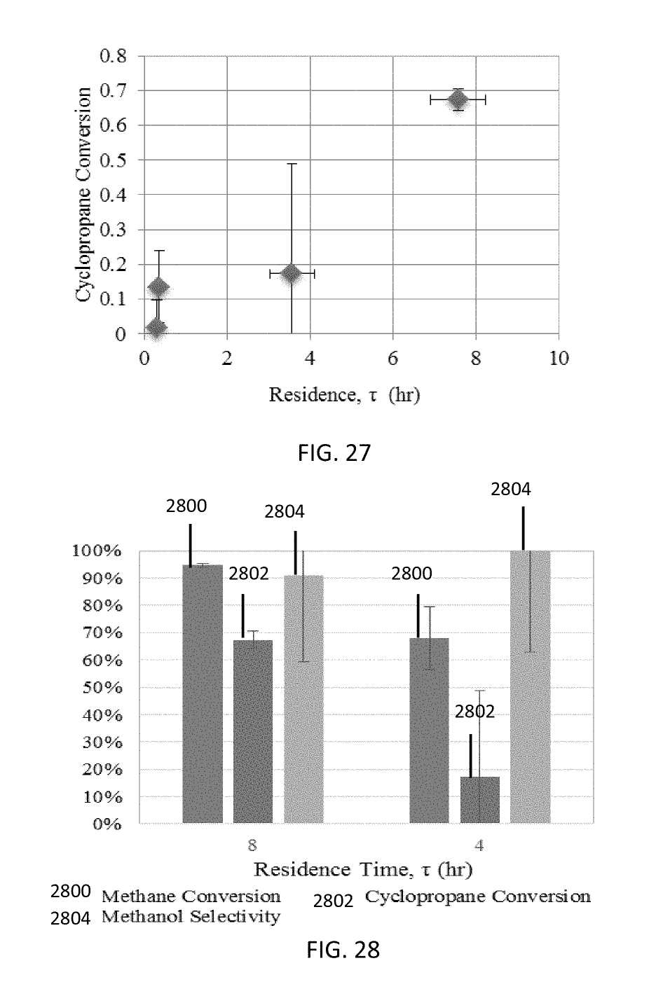

Cyclopropane concentrations decreased indicating it was being transformed with maximum conversions of 67±3% at τ of 8 hours and about 17%±32% after about 4 ours (FIG. 27). Methanol was detected at residences times of 4 and 8 hours when appreciable cyclopropane conversion was observed. Outlet concentrations were measured and evaluated against methane conversion on a mass balance basis to determine methanol production selectivity (percentage of methane converted to methanol). Methane conversion of 95±1% and 68±11% was achieved at reactor residence times of 8 and 4 hours, respectively representing a selectivity of 91±32% and 102±39 (FIG. 28). These initial results indicate that cyclopropane was transformed to make cyclopropanol and that cyclopropanol inhibited MDH.

It also will be determined whether cyclopropane needs to be continuously added, or whether it is an irreversible inhibitor, that can be periodically added to inhibit the MDH enzyme.

### Example 3

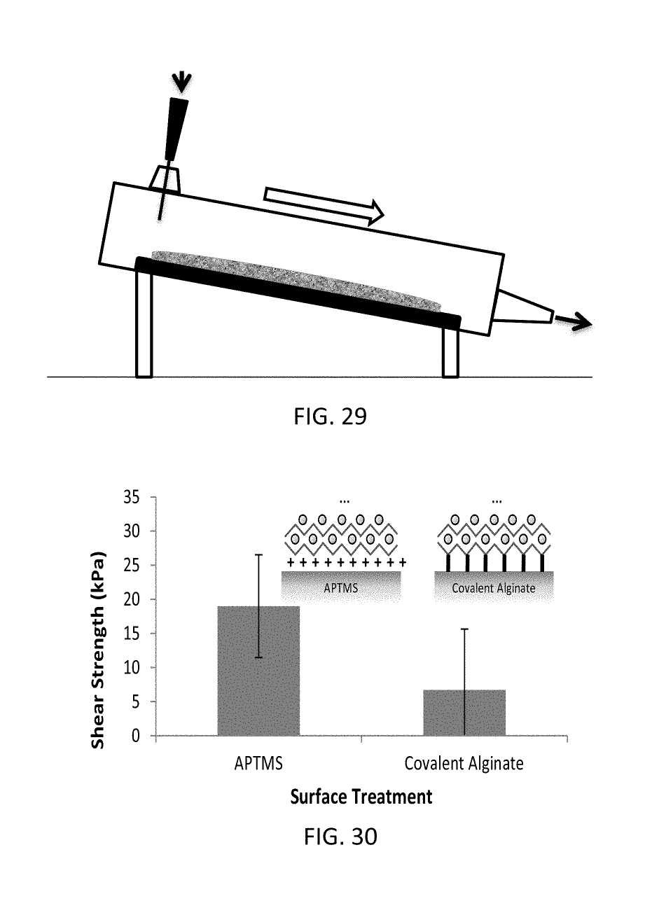

In this example, the stability of naturally-grown and immobilized biofilms of Methylosinus trichosporium OB3b is described. The naturally-grown and immobilized biofilms were evaluated in a four-channel drip flow reactor (FIG. 29, BioSurface Technologies, Inc., Bozeman, Mont.) on slides made of stainless steel treated in the same way as the bio-lamina substrates disclosed herein. Two DFR lanes represented a natural biofilm (NB) treatment and concentrated M. trichosporium OB3b was added directly to the slide. The other two DFR lanes had culture immobilized in alginate (AI, 2% final alginate concentration). Each DFR lane received a total of 7 mg cell protein. The DFR was incubated in batch mode at 30° C. for 4 days to allow the NB treatment time to attach to the stainless steel slide before 1/10-strength growth medium was fed at a rate of 14 mL h⁻¹ to each channel A mixture of methane (30%) and air was supplied by mass transfer from the headspace of the DFR channels at pressure slightly above atmospheric through 0.45 μm filters. Periodic tests of rates of ethylene to ethylene oxide conversion demonstrated that the DFR lanes that held the AI treatment consistently had rates of ethylene oxide production 300% greater than the NB treatment. The alginate in the AI treatments was stable for over three weeks under DFR conditions.

The breaking and adhesion strength of different gel formulations and surface treatments can be quantified to enable optimization of the biofilm formation method. In one example, thin, uniform 1″-square alginate gels were formed between SS shims treated with either aminosilane or a covalently-linked alginate “priming layer,” and then drawn laterally apart by an Instron mechanical tester. Alginate adhesion was greater on APTMS-SS than with an alginate priming layer (FIG. 30). Both treatments were superior to acid passivation alone.

In another example, a method to dissolve the alginate gel by chelation with sodium citrate was conducted to retrieve the encapsulated bacterial cells and stain them in suspension. Citrate is a metabolic intermediate, so is not expected to harm bacteria. Live and killed OB3b cells encapsulated in alginate were released with citrate. The live bacteria remained essentially uncompromised.

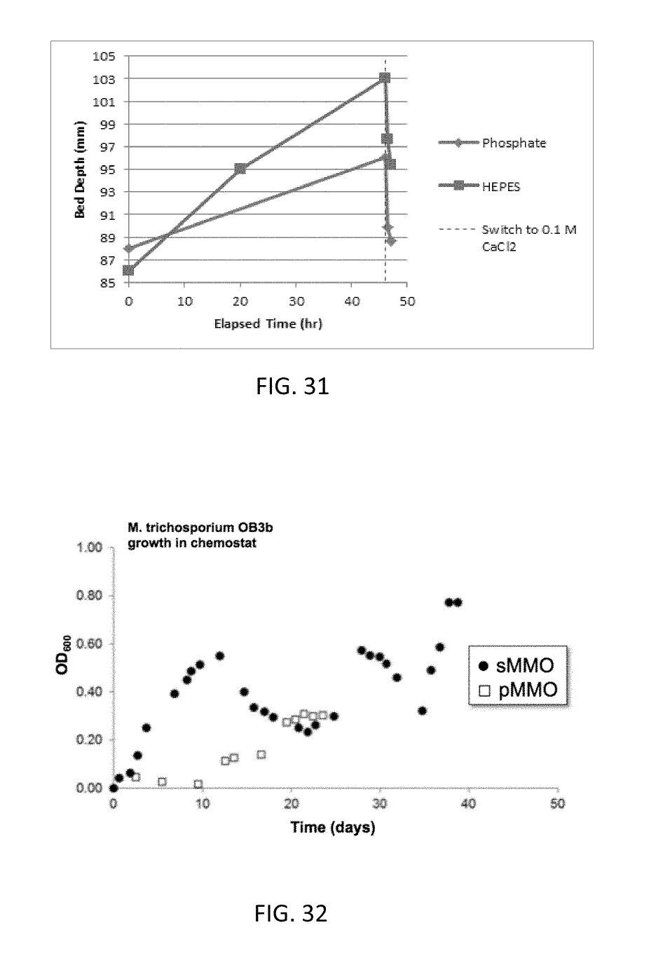

In one example, the biofilm integrity was evaluated after a multi-day demonstration run of the bio-lamina bioreactor. In some examples, any unanticipated loss of biofilm integrity may be caused by a combination of flow maldistribution, slow leakage of stabilizing Ca²⁺ ions from the biofilm caused by the flow, or continuous flow of the 1:10-diluted, Ca²⁺-poor growth medium flowing over the biofilm. In one example, a bolus of blue food dye was injected into the flow as a mechanism to detect any potential loss in biofilm integrity. The resulting blue color from this example occurred only in the biofilm at the periphery of the channel, confirming that the liquid flowed predominantly along the channel path. Samples of the biofilm taken from the unaffected regions of the plate retained their as-cast shape and integrity. Any deleterious effects caused by flow maldistribution, slow leakage of stabilizing Ca²⁺ ions from the biofilm caused by the flow, or continuous flow of the 1:10-diluted, Ca²⁺-poor growth medium flowing over the biofilm may be prevented by replacing suspect “troublesome” media components (e.g. KNO₃ and phosphate) with innocuous compounds (e.g. Ca(NO₃)₂ and HEPES buffer). Comparative results obtained from using phosphate or HEPES buffer are illustrated in FIG. 31. In some examples, mixtures of alginate and carrageenan can be used to stabilize the gel against competitive displacement of Ca²⁺ by monovalent (Na⁺/K⁺) ions in the liquid media stream.

In additional examples, methods to address any of the potential deleterious effects discussed above are disclosed. In some embodiments, the concentration of CaCl₂ in the media flowing in the reactor was increased to decrease the driving force for leaching of Ca²⁺ ions from the biofilm. In another embodiments, the internal gelation methods disclosed herein can be used to solidify the biofilm. The internal gelation produces biofilms with more uniform Ca²⁺ ion distribution and better mechanical properties than the “dip” method. Internal gelation also leaves nanoparticles of solid CaCO₃ within the gel, which serve as an internal source of Ca²⁺ to replace calcium ions as they are leached to the flowing media.

### Example 4

In this example, alternative immobilization strategies are disclosed for forming the biofilm. In one example, the reversible Ca²⁺-alginate associations can be augmented with permanent inter-chain chemical bonds. A method to chemically activate alginate —COOH groups using EDC/NHS chemistry can be used. In such a method, the activated chains form permanent crosslinks with lysine or amine-rich biopolymers (e.g. polylysine, chitosan, etc.) and further anchor the bulk gel on aminosilane-treated bio-lamina substrate surfaces.

In another example, physical anchoring of an encapsulating gel, or bacterial cells or biofilms, using microstructures such as SiO₂ nanosprings. The nanosprings will be seeded with live OB3b cells to allow them to form confluent biofilms within the open, porous nanospring matrix. Surface modifications based on chemistry similar to the aminosilane disclosed herein will also be employed to enhance the cells' adhesion to the nanosprings. In some embodiments, a combination of Al foil and nanosprings can provide a very inexpensive but extremely durable support for OB3b and/or the film-forming material (e.g., alginate), providing the ability to produce disposable bioactive inserts to be placed in reusable polymer or metal bio-lamina substrates. This will drastically decrease the unit cost of bio-lamina substrates, reduce storage size and weight for bio-lamina substrates, and enable rapid and simple deployment of a wide variety of biocatalytic functions in a single bio-lamina bioreactor.

### Example 5

In one embodiment, a seven-liter chemostat was used to grow a culture of M. trichosporium OB3b without copper present, therefore expressing sMMO. The temperature-controlled jacketed reactor has multiple input and output lines for gas and liquid feed, a paddle stirrer for agitation, sensors for continuous monitoring of pH and DO, and metered oxygen delivery tied to a DO concentration set-point. The reactor was inoculated with a dilute culture of M. trichosporium OB3b and operated in batch mode for approximately one month before continuous fluid flow was established. Problems with inconsistent gas delivery resulted in a plumbing change around day 45, where oxygen delivery to the chemostat was linked to continuous DO monitoring and metered O₂ delivery to attain a DO set-point concentration. A second two-liter chemostat growing a culture of M. trichosporium OB3b with copper present, expressing pMMO, also was used.

Each reactor was initiated by adding a dilute inoculum of the culture in growth media to each respective reactor. Oxygen and methane were continuously supplied to the reactor. The reactor operated under copper limitation (sMMO expression) was started first and operated under batch flow conditions until an OD of approximately 0.6 was achieved (FIG. 32, ˜12 days). Continuous feed of fresh growth media was then started, resulting in a significant reduction in culture OD in the reactor. Flow was stopped and analysis of reactor conditions showed alternating periods of high methane, low oxygen concentrations with periods of low methane, high oxygen concentrations revealing inconsistencies in gas delivery to the chemostat resulting in culture growth limitations. After efforts to improve stability of gas delivery, continuous media flow was again started and produced very similar results (FIG. 32, d25-30). The gas delivery can be altered so that both methane and oxygen will be delivered with a positive displacement system to ensure adequate gas substrate delivery to the culture. Given the low aqueous solubility of both methane and oxygen, mass transfer of gaseous substrates is expected to limit growth in the chemostat systems. The modified gas feed system should ensure reliable gas delivery and quantifiable uptake rates.

In some examples, the 2-L reactor operated with copper present (pMMO) also experienced growth instabilities under continuous-flow operation. Aqueous media flow was stopped and the culture reached an OD of approximately 0.3 while operated in batch mode.

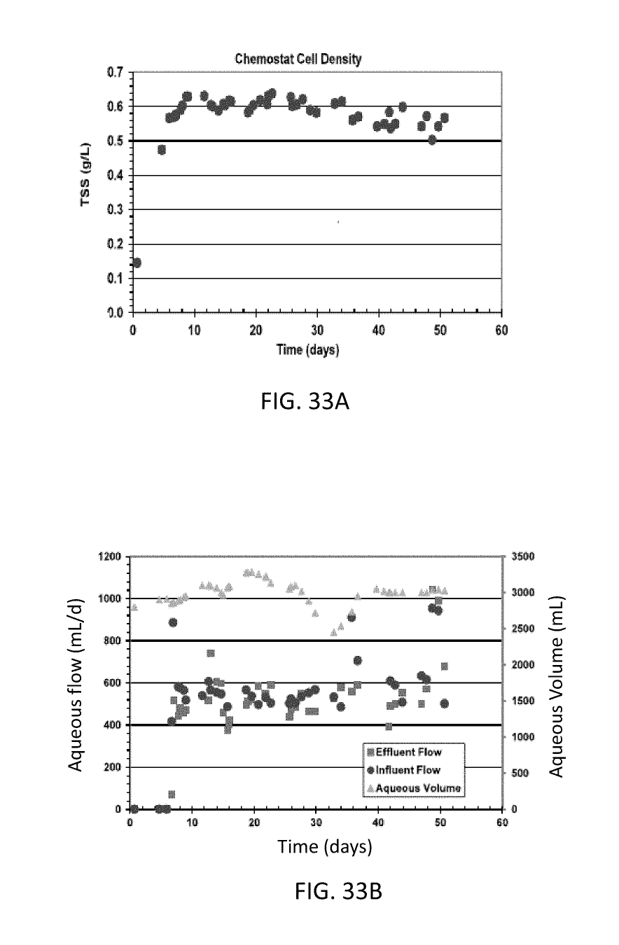

The chemostats were operated in continuous feed mode intermittently. As established below, consistent growth has been achieved in sequential batch cultures due to the increased control over oxygen and methane proportioning. In some embodiments, cell density was a consistent chemostat parameter over the operational period with an average concentration of 590 (±32) mg TSS/L (FIG. 33A).

More consistent methane and air delivery greatly improved chemostat function and the chemostat can be used as the source of cells used for the bio-lamina bioreactors disclosed herein. Preparation of each biofilm is expected to utilize from 2-5 g of active biomass. In some examples, a target biomass concentration in the chemostat was 1 g TSS/L.

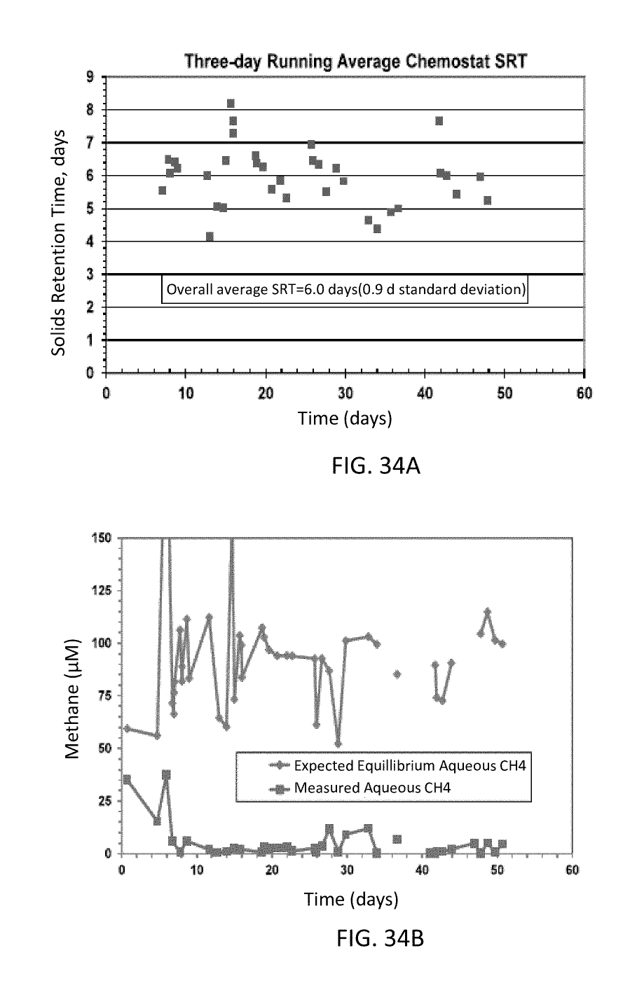

Separate pumps are used for the influent and effluent flows. In some cases additional cell harvesting occurs resulting in some variability in the solids retention time in the chemostat. FIG. 33B shows the influent and effluent flows and the chemostat aqueous volume over time in the chemostat. The variation in fluid flows and chemostat volume resulted in a range of operational solids retention times rather than one constant value as can be seen in FIG. 34A. Although the overall chemostat performance maintains a pseudo-steady state condition, metabolic activity within the chemostat varies with the actual amount of biomass wasted daily. The chemostat is mass transfer limited and operates with little or no methane remaining in aqueous solution while considerable methane remains in the effluent gas stream (FIG. 34B). Mass transfer limitations mostly result in upper limits on metabolic activity rates and a lower steady-state biomass concentration, but should not significantly affect the ability of the organisms to produce methanol when an inhibitor is added.

The reactivity tests developed to monitor metabolic activity have been applied to the chemostat culture during the period of pseudo-steady state operation. Table 1 lists the average values and standard deviations for the various metabolic activity tests conducted on the chemostat culture. Methanol-based SOURs and total biomass concentration were the most stable parameters with coefficients of variation near 5%. Methane oxidation rates within the chemostat measured from the difference of influent and effluent methane exhibited the highest variability in the activities measured. This is most likely due to the discreet nature of the sampling (few times per day) versus the dynamics in methane concentration and flows in the influent and effluent gases. Batch test methane and ethylene oxidation rates conducted on cells harvested from the chemostat were more consistent, but still exhibited significant variability. Average methane oxidation rates in batch assays reasonably closely matched the values obtained from direct chemostat measurements.

TABLE 1

| | Chemostat | | Methane | Ethylene | | | | |
|---|---|---|---|---|---|---|---|---|
| | Methane | | Oxidation | Oxidation | Methanol | Formate | | Net |
| | Oxidation rate | | Assay | Assay | SOUR | SOUR | Total Biomass | Growth |
| | | mmol/hr/g | mmol/hr/g | mmol/hr/g | mmol/hr/g | mmol/hr/g | Concentration | Rate |
| | g/L/hr | TSS | TSS | TSS | TSS | TSS | g TSS/L | mg/hr |
| Average | 0.015 | 1.57 | 1.21 | 0.59 | 1.20 | 0.51 | 0.59 | 13.5 |
| σ | 0.009 | 1.03 | 0.31 | 0.26 | 0.084 | 0.092 | 0.032 | 2.8 |
| Coefficient of | 62 | 66 | 25 | 44 | 7 | 18 | 5 | 21 |
| variation (%) | | | | | | | | |

The variability in the activities measured are normal and appear to be related to the amount of methane introduced to the reactor each day in comparison with the total biomass contained in the bio-lamina bioreactor. In some examples, an inhibitor is added to the chemostat to cause the accumulation of methanol within the reactor. Although subtle metabolic changes in the chemostat will be covered by the data variability, inhibition to cause the accumulation of methanol is expected to result in significant reduction in methanol dehydrogenase activity while having little effect on methane oxidation rates and should be quantifiable in metabolic activity tests.

Exogenous formate (20 mM) has been to the chemostat aqueous feed for the last two weeks and was added in expectation that the inhibited cells would need the formate to eliminate MMO rate limitation due to loss of reducing power. Additionally, formate SOURs have been conducted to estimate resting cell formate oxidation rates and to quantify formate oxidation activity over time. However, preliminary batch tests conducted with alginate-encapsulated cells indicate that formate-grown cells recover from cyclopropanol inhibition significantly faster than non-formate-grown cells. Therefore, formate has been eliminated in the chemostat feed in attempt to provide the most favorable conditions for methanol accumulation in the chemostat. Due to the washout dynamics of the chemostat, methanol will remain in solution for a significant amount of time (HRT=6 d) before leaving in the chemostat effluent flow.

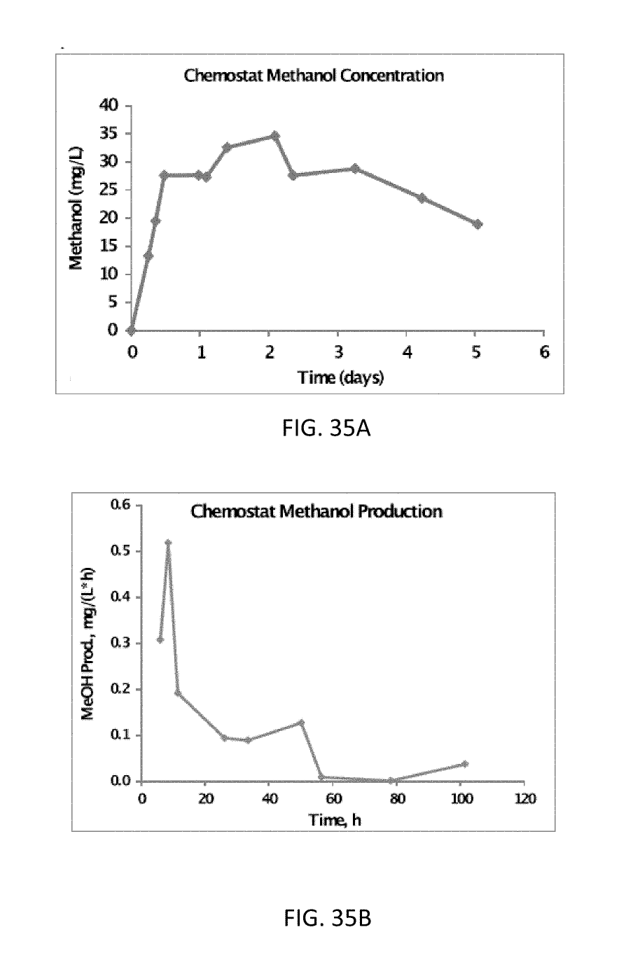

Once the chemostat was operating at pseudo-steady-state conditions, cyclopropanol was introduced to selectively inhibit methanol dehydrogenase (MDH) function and result in methanol accumulation in solution. Cyclopropanol was added under batch-flow conditions with no methane being fed to the reactor to limit methanol competition for MDH and increase the effectiveness of the inhibitor. Methanol accumulated in solution for about 24 hours and then was slowly washed from the reactor (FIG. 35A). The microbial methanol production rate in the chemostat peaked within 24 hours at about 0.5 mg/(L*h) and was essentially zero after two days (FIG. 35B). Post-inhibition microbial activity sampling revealed a 94% reduction in batch methane oxidation rate, a 97% reduction in ethylene oxidation rate, a 95% reduction in methanol-dependent oxygen uptake rate, but only a 20-25% reduction in formate-dependent oxygen uptake rate.

The activity results remained stable at the above values for 5 days after inhibition with cyclopropanol present throughout. Biomass concentration within the chemostat was observed to follow a path consistent with washout of an inert substance from the reactor, as did cyclopropanol. Complete shutdown of methane oxidation within the chemostat most likely occurred due to very successful inhibition of MDH resulting in a loss of reducing power needed for MMO operation. On day 5, formate was added to the chemostat feed to determine if it was sufficient to re-activate methane utilization, since formate-dependent oxygen uptake rates indicated that formate could still be processed by the inhibited culture. By day 8, methane utilization and oxygen uptake were restored in the chemostat and the biomass concentration increased back to pre-inhibition values. Successful methanol production was demonstrated within the chemostat, but was not continuous as it ceased after only 24-48 hours. The chemostat inhibition experiment is being conducted again with modifications to the concentration of inhibitor and conditions of inhibition in attempt to produce conditions for continuous methanol production and to maintain higher methane oxidation rates throughout.

In some embodiments, M. trichosporium OB3b culture was immobilized in alginate beads. M. trichosporium OB3b culture expressing sMMO was harvested and suspended to a final density of 10 g cell protein/L in 2% alginate. The alginate/cell mixture was extruded through a syringe needle into 100 mM CaCl₂ to crosslink the alginate matrix, forming stable beads. Beads were rinsed with dH₂O and re-suspended in 1/10 strength (dilute) growth media. M. trichosporium OB3b culture prepared this way is ideal for experimental manipulation such as CH₄ or MeOH rate determinations after inhibitor exposure because beads can be quickly rinsed to remove trace inhibitor. OB3b culture immobilized in alginate in this way maintained activity for >7 days.

Inhibition of Methanol Consumption with Cyclopropanol

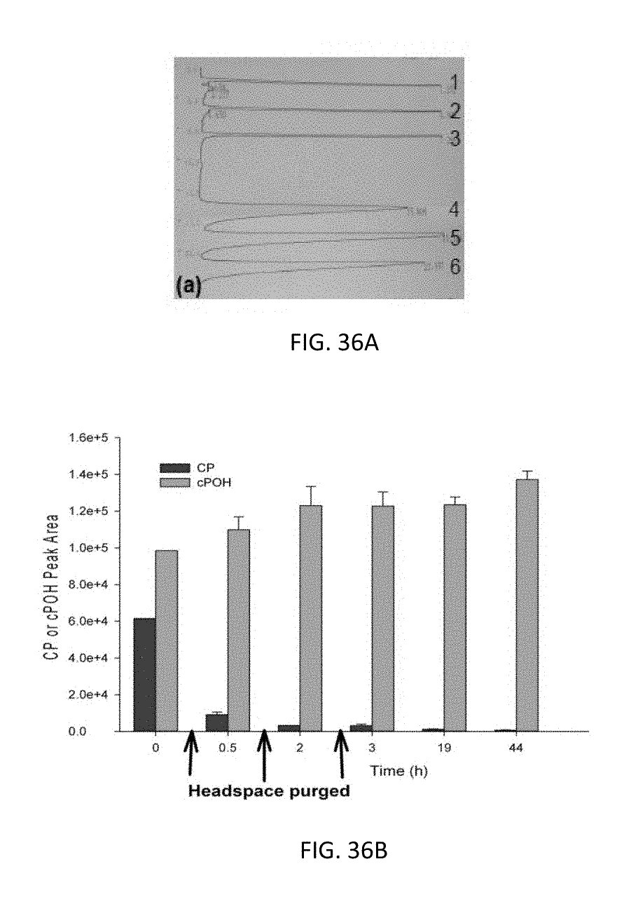

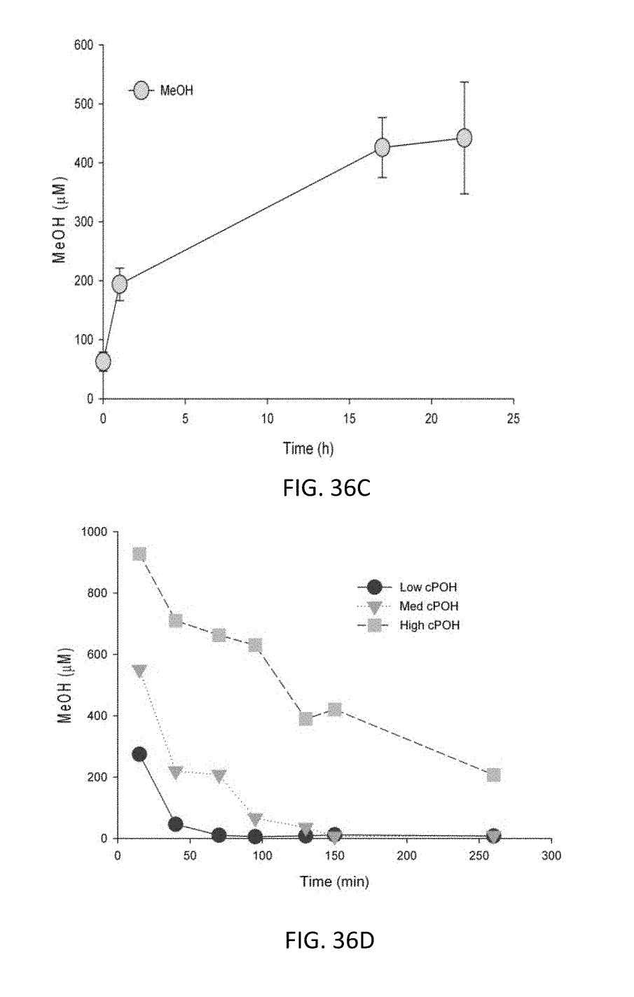

In the absence of CH₄, sMMO expressing OB3b in alginate beads were incubated 18 h with cyclopropane (CP, FIG. 36A) and produced an oxidized product hypothesized to be cyclopropanol (cPOH). cPOH is more water soluble than CP, enabling removal of CP by purging the headspace with air (FIG. 36B). Work is progressing on the identification cPOH by mass spec analysis. After the M. trichosporium OB3b culture produced the cPOH was rinsed in dilute media and incubated with CH₄, MeOH accumulated for 18 h suggesting inhibition of methanol dehydrogenase (MDH, FIG. 35C). Inhibition of MDH was confirmed by exposing fresh non-inhibited sMMO expressing M. trichosporium OB3b to cPOH (FIG. 36D).

In the absence of cPOH 1 mM MeOH was consumed <10 minutes, but when cPOH was added with 1 mM MeOH there was a concentration dependent effect on the rate of MeOH consumption. At low and medium cPOH concentrations, significant amounts of MeOH were consumed before the initial time points could be taken. However, the highest concentration of cPOH significantly slowed MeOH consumption. The lack of complete MDH inhibition by cPOH was the first example that showed that exposure to cPOH or CP for the inhibition of MDH needed to occur in the absence of CH₄ or MeOH in order to maximize the inhibition.

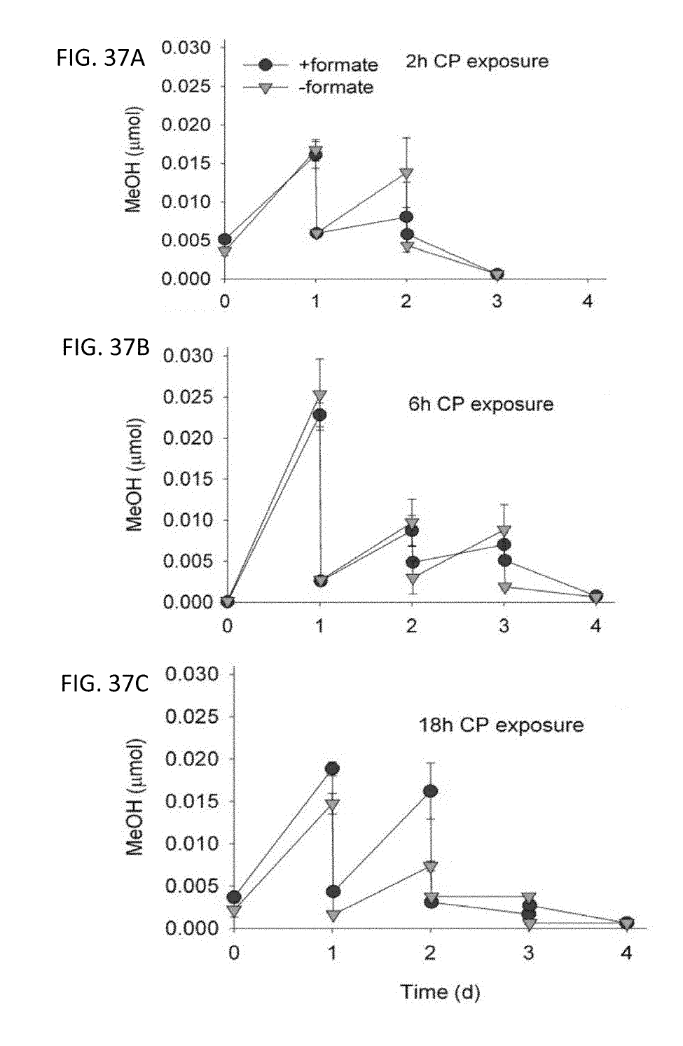

A chemostat grown, sMMO expressing, M. trichosporium OB3b culture was immobilized in alginate beads, and exposed to CP for 2, 6, and 18 hours in the absence of CH₄. After inhibition, the consumption of CH₄ and accumulation of MeOH was monitored in the presence and absence of 20 mM formate in sequencing batch reactors (FIGS. 37A-37C). Alginate beads were rinsed, at 24 hour intervals, before suspension in fresh media plus CH₄. There was no significant difference: i) in the rate of methane consumption in the treatments that were exposed to CP for 2, 6 or 18 h (p>0.5, 30-80 μmol CH₄ consumed/d, Table 2); ii) in the efficiency of MeOH accumulation in any of the treatments (Table 2); and, in the effect of formate on MeOH accumulation (P>0.18) for any exposure time, probably due to lack of complete MDH inhibition (Table 3). It was also observed that MeOH accumulated to a greater degree during days 2 and 3 in treatments after 6 or 18 hours of CP exposure. Further refinement of MDH inhibition by CP needs to be achieved, as MeOH consumption was significantly slowed by not completely inhibited.

TABLE 2
Effect that length of CP exposure has on MeOH accumulation
efficiency during initial 24 hours of incubation with CH₄.

| Length of CP exposure (h) | MeOH Accumulation Efficiency (%) |
|---|---|
| 2 | 19 (4) |
| 6 | 43 (1) |
| 18 | 13 (1) |

(Efficiency = MeOH accumulation/CH₄ consumed)

TABLE 3
Rates of MeOH consumption before and after CP exposure

| | Rates of MeOH consumption | |
|---|---|---|
| | Pre exposure | Post Exposure |
| Length of CP exposure (h) | (μmol/h/mg protein) | |
| 2 | 1.0 (0.0) | 0.4 (0.0) |
| 6 | 1.2 (0.3) | 0.6 (0.1) |
| 18 | 1.4 (0.1) | 0.3 (0.1) |

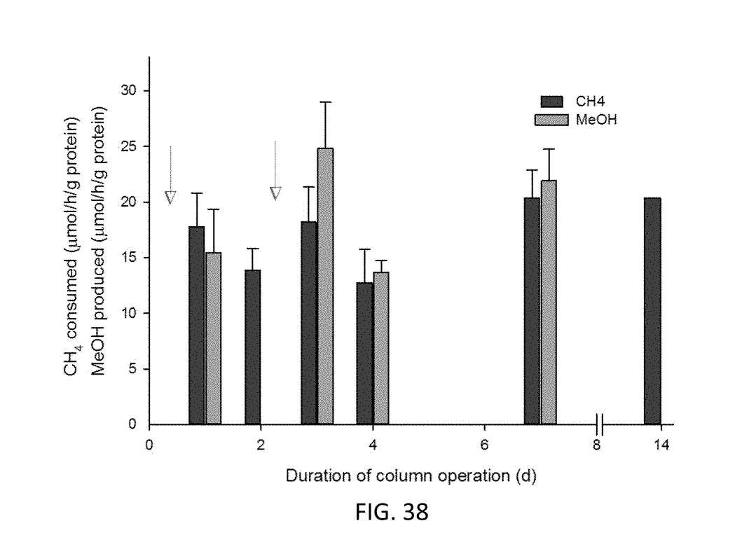

In parallel with the BLP reactor runs, the response of methane consumption and MeOH production in a column packed with M. trichosporium OB3b culture immobilized in alginate beads (0.4 mg cell protein total) was evaluated. The column was incubated under batch conditions for 18 hours with cPOH, then quickly flushed with ˜10 pore volumes of dilute growth media, before dilute media flow containing ˜200 μM CH₄ was begun at a rate of 41 ml/hour (FIG. 38). Over the course of day 1, 80±10% of influent CH₄ was consumed and MeOH accumulated with an efficiency (MeOH produced/CH₄ consumed) of 94±21%. During day 2, 67±6% of influent CH₄ was consumed but no MeOH accumulated. To see if MeOH production could be renewed, the column was again exposed to cPOH for 18 hours as previously described. On days 3, 4, and 7, 66±8% of influent CH₄ was consumed and MeOH accumulated with an efficiency of >90%. After a week of no media flow conditions the majority of influent CH₄ was consumed indicating that alginate immobilized M. trichosporium OB3b could maintain activity for long periods of time.

As seen in FIG. 32, a higher cell density was achieved in the chemostat growing the cell culture expressing sMMO, although the cell density was more unstable, when compared to the chemostat growing the cell culture expressing pMMO. Once stable operation is established in the chemostats, activity tests can be conducted to evaluate baseline methane and oxygen utilization rates before evaluating inhibition. M. trichosporium OB3b cultures can be growing successfully under plus copper (pMMO expressed) or minus copper (sMMO expressed) conditions in batch cultures. New stock cultures can be initiated weekly from the previous active stocks that have no growth on heterotroph check plates (LB agar). Working cultures also can be initiated weekly (1% inoculum) from active heterotroph-free stocks. After initial 72 hour growth, cultures are diluted daily (2-fold) with fresh media, bottle headspace refreshed, and methane added.

M. trichosporium OB3b cultures were grown under plus copper (pMMO expressed) or minus copper (sMMO expressed) conditions using chemostat or batch culture conditions. Aliquots of culture (˜100 ml) were harvested by centrifugation, and concentrated 50-fold in fresh media. Pelleting cells in this manner ensured that all residual methane was eliminated, increased ease of measuring protein, and made activity assays as brief as possible. Assays were performed in 30 ml crimp cap reaction vials with 4 ml of media (either +Cu or −Cu) with 10 mM sodium formate. To initiate assays, 100 υl of the 50× cell suspension was added to reaction vials with a plastic syringe. Short descriptions of activity assays follow.

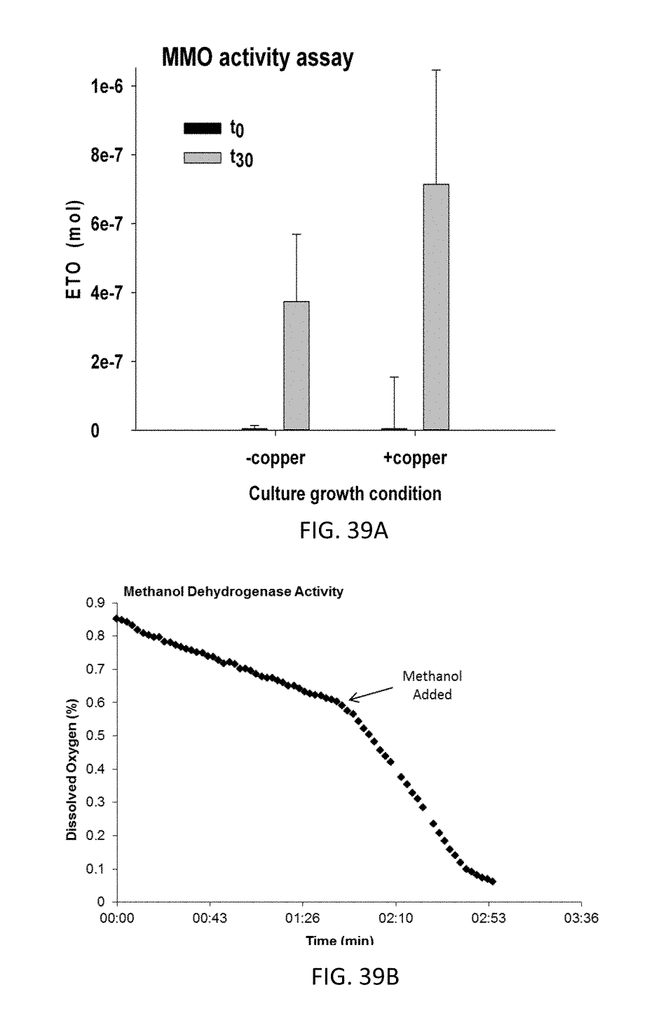

Due to the relative insolubility of methane, large volumes should be added to reaction vials to achieve concentrations in the aqueous phase (C_{w}) above the concentration of methane where the velocity of methane consumption (V) is equal to 0.5Vₘₐₓ for minus copper grown M. trichosporium OB3b cultures. These large methane additions make direct measurement of methane consumption in short-term assays unlikely, therefore the accumulation of ethylene oxide (ETO) upon the oxidation of ethylene (ETH) was used as a surrogate measurement. Reaction vials were prepared as described above and 5 ml of ETH added as an overpressure. The appearance of ETO was monitored by gas chromatography evaluation of C_{w} over the course of 30 minutes (FIG. 39A). Killed cells (boiled) served as a negative control. pMMO is a copper enzyme and can be inhibited by allylthiourea (ATU), while sMMO (an iron enzyme) is insensitive to inhibition by ATU. The ETH to ETO assay described above was repeated with 10 μM ATU to discriminate between sMMO and pMMO activity.

The procedure for measuring methanol dehydrogenase (MDH) activity by measuring the specific oxygen uptake rate (SOUR) has been tested on M. trichosporium OB3b cultures grown both in batch and in the chemostat. M. trichosporium OB3b cells were harvested using centrifugation. The supernatant was poured off and the cells were re-suspended in fresh media lacking a carbon source. Phosphate buffer solution was added to a sample chamber in which dissolved oxygen (DO) was measure with a DO probe. When the DO of the solution stabilized, the re-suspended cells were added to the sample chamber to measure baseline oxygen uptake by the cells without a carbon food source present. Methanol was then added to test for MDH activity. The difference in the oxygen uptake rates illustrates the MDH activity of the culture. The results of the MDH activity test on M. trichosporium OB3b grown in batch under minus copper conditions is shown in FIG. 39B.

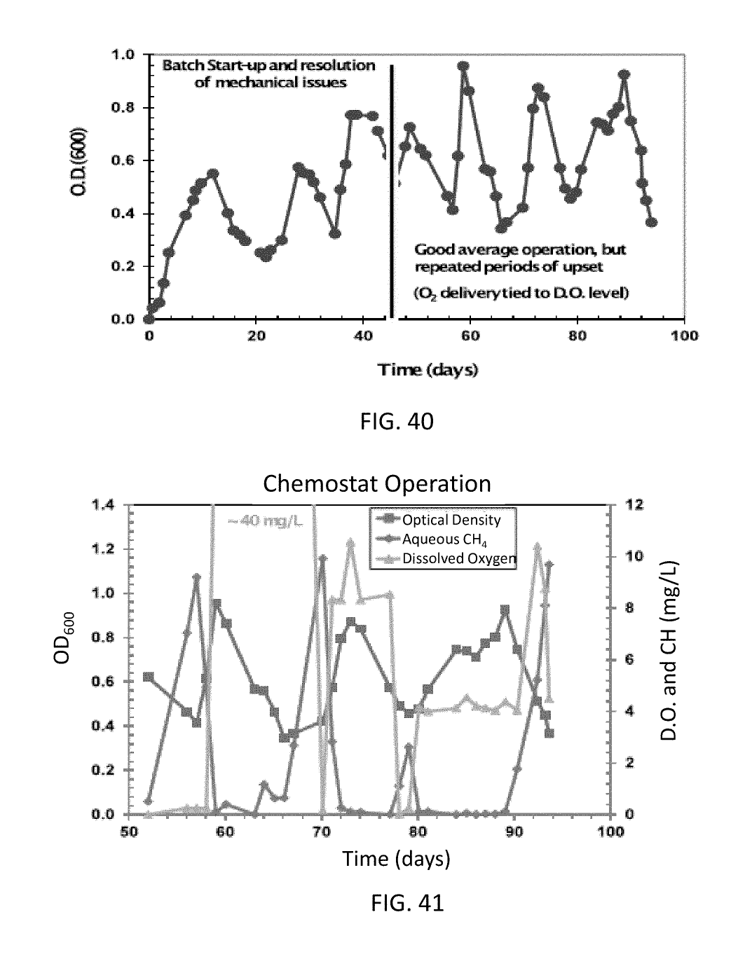

In some examples, semi-stable operation was achieved, producing an average methane oxidation rate of about 20 mg CH₄/(L*hr) at an average OD₆₀₀ of 0.6 (or approximately 400-500 mg dry wt/L). Operation in the semi-stable period shows periodic upsets, or cycles of high and low growth (FIG. 40). The periodic growth upsets appeared to be associated with periods of high oxygen delivery/concentration in the chemostat. The DO set point was serially lowered until stable operation at a DO concentration of 4 mg/L was achieved (FIG. 41, day 80-90), until DO sensor failure on day 89 caused excessive oxygen delivery to the chemostat. Routine plating of the chemostat culture showed significant heterotrophic contamination at that point, so the chemostat was re-plumbed, re-inoculated, and re-started. The chemostat was plumbed for continuous metered oxygen and methane delivery (not tied to reactor concentration set-points), was re-inoculated with a dilute culture of M. trichosporium OB3b expressing sMMO, was incubated in batch mode for 3 days and can operate with continuous flow under stable conditions for longer time periods.

Metabolic activity test methods have been developed to monitor specific activities in M. trichosporium OB3b cultures. A naphthalene oxidation assay was used to determine if sMMO is being expressed. An ethylene to ethylene oxide reaction assay and a methane oxidation assay was used to quantify MMO activity, and a methanol oxidation assay was used to estimate MDH activity. Concurrent batch metabolic tests can be conducted to investigate methanol inhibition of methane oxidation, cyclopropane to cyclopropanol conversion and concurrent/subsequent cyclopropanol inhibition of MDH activity, the effect of cyclopropanol inhibition on growth yield, and the effects of high oxygen concentration. Once stable operation and baseline metabolic monitoring in the chemostat have been achieved, inhibitor can be added to evaluate chemostat operation under methanol-producing conditions.

Distinguishing Between sMMO or pMMO Activity:

The sMMO system has a broad substrate range and will oxidize naphthalene to naphthanol, while pMMO-expressing cultures cannot. Naphthanol in turn reacts with tetra-azotized o-dianisidine (Fast Blue dye) yielding a pink color. This simple assay was used to determine if cultures were expressing sMMO or pMMO before experimental manipulation.

Methane Monooxygenase (MMO) Activity:

MMO activity has been measured via both methane (CH₄) consumption and ethylene oxide (ETO) production upon oxidation of the non-growth substrate ethylene (ETH, Table 4). While ETO production matches methane consumption for pMMO, ETH is not as good a substrate for sMMO as methane. Nevertheless, the ETO accumulation assay is quick (<30 min) and can be used to screen for activity before starting a larger experiment.

TABLE 4

| | Eth to ETO | CH₄ |
|---|---|---|
| | [nmol/min/mg protein] | |
| sMMO | 13(0.1) | 153(9) |
| pMMO | 75(42) | 65(28) |

Inhibitor Trials:

A selective inhibitor of MDH activity that does not substantially reduce MMO activity is desired for some embodiments. The effect of salts NaCl, NH₄Cl and CaCl₂ on MMO and MDH activity was evaluated using the reactivity assays described above. None of the salts were effective in selectivity inhibiting MDH activity, and an example of those results is shown in Table 5. sMMO was more sensitive to inhibition by the tested salts than MDH, and methanol did not accumulate.

TABLE 5

| | Fraction of Activity Remaining | |
|---|---|---|
| CaCl₂[mmol] | sMMO | MDH |
| 10 | 0.41 | 0.71 |
| 20 | 0.29 | ND |
| 40 | 0.20 | 0.45 |
| 66 | ND | 0.35 |

TABLE 6

| | Fraction of Activity Remaining | |
|---|---|---|
| CP[μmol] | sMMO | MDH |
| 0 | 1.0 | 1.0 |
| 03 | 0.9 | 0.9 |
| 32 | 0.9 | 0.2 |
| 64 | 0.8 | 0.1 |

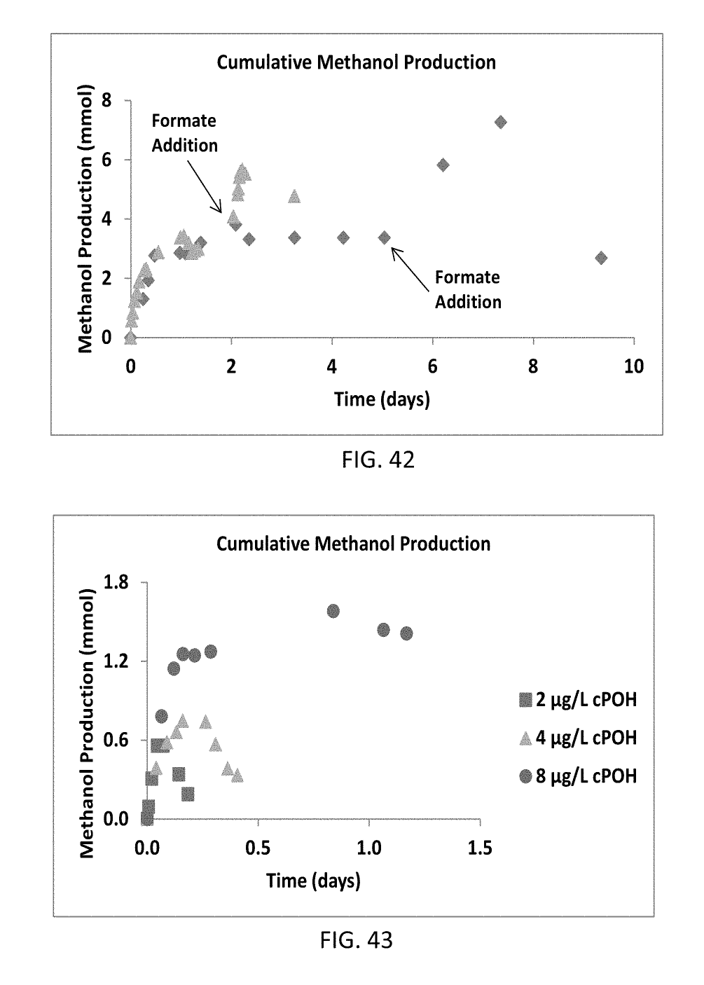

In some examples, initial attempts at producing methanol in the chemostat resulted in near complete inhibition of both methanol and methane oxidation. In the first two embodiments an estimated 75 μg/L cyclopropanol (cPOH) was added to the chemostat. Methanol production by M. trichosporium OB3b within the chemostat lasted less than 12 hours. Following initial methanol production, cell growth ceased and cells began washing out of the reactor. Once signs of washout were observed formate was added to the chemostat to a concentration of 10 mM. The addition of formate resulted in short term production of methanol followed by apparent recovery of methanol oxidation capability (FIG. 42). In subsequent attempts at methanol production, smaller cyclopropanol additions were used in an attempt to inhibit the organisms enough to produce methanol without disrupting their ability to grow fast enough to avoid washout. Methanol production was observed for about 2-6 hours following the addition of 2-8 mg/L cyclopropanol. In each case, a peak in cumulative methanol production was reached followed by a decline indicating the recovery of the methanol oxidation capability of the culture (FIG. 43). The duration of the peak in cumulative methanol production increased with increasing cyclopropanol concentration, indicating that the time required for recovery of MDH function is dependent on the cyclopropanol concentration used. Since the cyclopropanol concentrations used in these examples were below the detection limit of the analytical method, the cyclopropanol fate in the chemostat is uncertain in some embodiments.

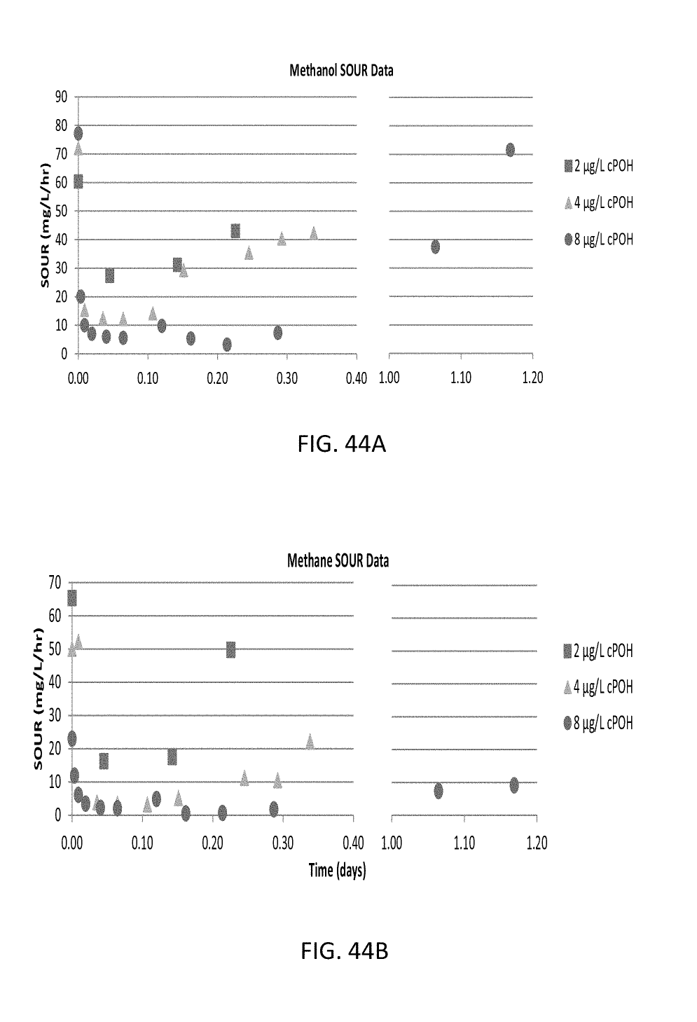

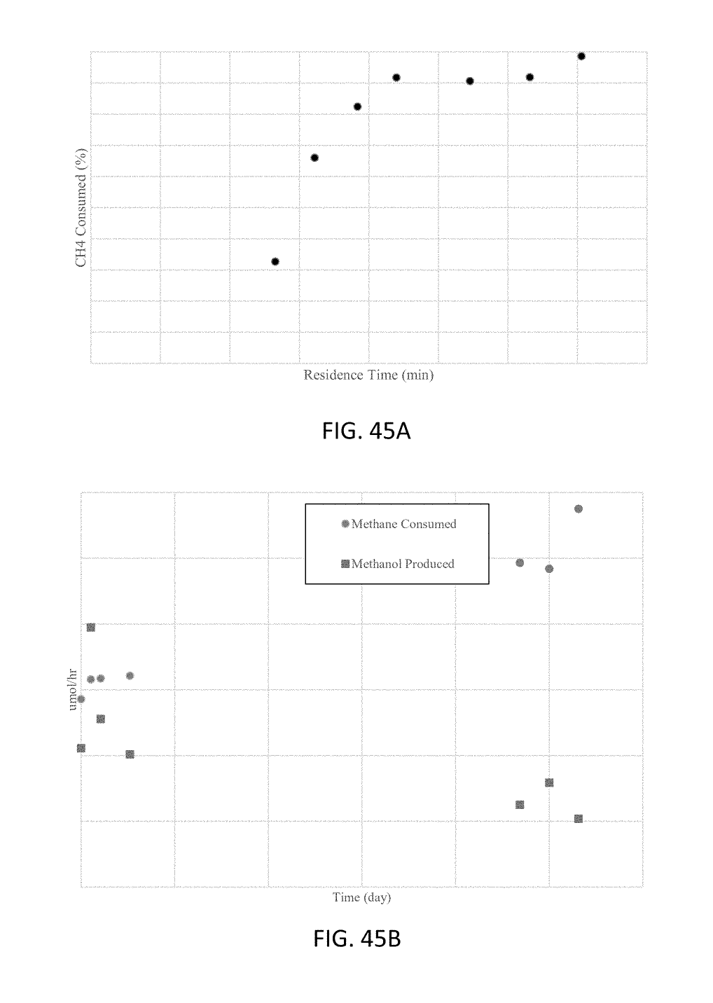

Throughout each experiment specific oxygen utilization rate (SOUR) tests were conducted with samples from the chemostat as indicators of MDH and methane monooxygenase (MMO) activity (FIGS. 44A and 44B). Methanol SOURs indicate essentially immediate inhibition of MDH activity, although it took up to 1 hour to reach a minimum in activity. Significant methane inhibition is indicated in the methane SOURs and it occurs with essentially the same magnitude and time scale as the methanol SOUR results. The recovery of MDH indicated by SOUR tests correlated with the observed reduction in cumulative methanol production in the chemostat. The recovery of MMO activity lagged behind MDH recovery, which was consistent with the observed reduction in methanol concentration in the reactor and indicates that at least part of the reduction in MMO activity was associated with a restricted re-supply of reducing power to MMO from metabolic processes downstream of methanol oxidation. Real time monitoring of pH and dissolved oxygen (DO) within the chemostat revealed patterns highly correlated with the methanol and SOUR data presented above, but offset back small time intervals. A peak in DO concentration was reached first followed by a peak in pH and lastly methanol concentration. The increase in pH and DO is expected to be the result of incomplete methane oxidation and a subsequent reduction in CO₂ production. In this case, where the concentration of cPOH is too low for analytical detection, in situ pH and DO monitoring allows for rapid assessment of the effects of cPOH addition. cPOH additions to the chemostat to reach 0.5 μg/L or lower cPOH concentration resulted in no discernable effects on pH and DO concentrations within the chemostat. This indicates that pH may be a valuable analysis for the BLP effluent to assess the extent of methane oxidation to CO₂.

Repeated addition of cPOH resulted in repeated production of methanol within the chemostat. To maintain a constant biomass within the chemostat, full recovery of the bacteria was required prior to re-inhibition by cPOH, which resulted in oxidation of the methanol already produced. SOUR tests indicated that MMO activity took longer to recover from energy depletion than MDH activity took to recover from cPOH inhibition. The data suggests the presence of formate can provide the energy required for MMO activity, although it may result in quicker recover from cPOH inhibition of MDH as well.

### Example 6

In this example, the rate of ETO accumulation for cells encapsulated in alginate versus those free in suspension was evaluated. Briefly, a sMMO-expressing culture was harvest by centrifugation and re-suspended in dilute (1/10 strength) minimal media (DMM). Aliquots of cell suspensions were added to vials with DMM or mixed with alginate (2% final concentration) and extruded through a hypodermic needle with a syringe pump to make beads. The alginate beads were stabilized in 100 mM CaCl₂, rinsed three times with DMM and then suspended in DMM. Assays were initiated by the addition of ethylene, and the rate of ETO accumulation monitored. ETO accumulated in alginate encapsulated and free cell suspensions at rates of 10.4±0.8 and 8.3±1.4 nmol/min/mg protein, respectively. There was no significant difference between the rates (p=0.09). The alginate beads with cells were also incubated with naphthalene and then treated with Fast Blue dye. The pink color indicative of sMMO activity appeared in the supernatant, and also in the beads themselves. The indicator color in the beads demonstrates that the cells were actively expressing sMMO within the alginate.

In some examples, OB3b cells were harvested from the chemostats described herein to make alginate beads for a long term packed column experiment. Total cell mass within the column was 300 mg with and the column void space was 6 mL. The flow rates of a methane solution (2 mg/L) were varied and the inlet and outlet concentrations were measured for a methane consumption efficiency curve shown in FIG. 45A.

An increase in methane consumption with an increase in residence time was observed. The column was then inhibited with cyclopropanol (1/10 dilution) for 18 hours. A flow rate of 2 mL/min was used for all further testing. Methane consumption and methanol production were monitored over 24 hours and the results are shown in FIG. 45B. Methane consumption decreased from 0.21 μmol/hr to 0.15 μmol/hr after the inhibition, while 0.13 μmol/hr of methanol was produced. After 24 hours methanol production decreased to 0.06 μmol/hr and methane consumption increased to 0.24 μmol/hr. The inhibition process was repeated for 18 hours and methanol production evaluated under the same operating conditions. The maximum methanol production rates lasted about 3 hours, and then declined, while methane consumption was maintained. Overall, the column responded similarly to two inhibitions ran at constant flow for two weeks with little problem.

### Example 7

In this example, the operation of a bio-lamina bioreactor embodiment is described. The parameters used in this example are described below.

| Parameter | Functional Criteria |
|---|---|
| Reaction Temperature | 20-35 [° C.] |
| Reaction Pressure | 20 [bar] |
| Water flow rate | 8.30 [mL/min]* |
| O2 flow rate | 2.80 [mL/min]⁺ |
| CH4 flow rate | 1.40 [mL/min]^{#} |
| Methanol production | 1.14 [g/L] |
| Residence Time | Operating flow rate dependent |
| Construction Material | 316 Stainless Steel |
| BLP Sealing Mechanism | O-ring seals around reactor perimeter |

*determined from the functional criteria of 25 [L water/L reactor/hr] flow requirement for reactor

In some embodiments, the device is designed to support internal bioreactor pressures up to 20 [bar]. The top and bottom clamp plates are used as an external shell clamp system to provide this support. Also, a high and robust surface area for coating with the biofilm is provided along with the structural projections to maintain the integrity of the supported biofilm. Hermetic sealing also is used around reactor perimeter to avoid leaks under reaction conditions. Swagelok fittings are used for input and output fluidic ports. Slug flow hydrodynamics are desired for the microscale two-phase flow regime. High interfacial area for mass transport is achieved in this flow regime. To provide a way to deliver consistent gas flow and liquid flow that would not be affected by variations in downstream pressures, small gas hole(s) for delivery (<10 [μm]) are machined by laser micromachining. Shallow channels for fast liquid flows to break gas flow into bubbles are produced by laser ablation micromachining methods (FIGS. 15 and 16). Characteristics depths (e.g., 360 μm for the fluid flow lamina and 460 μm for the biofilm lamina) are selected to ensure fast mass transport times from fluid side to bio film side.

The bio-lamina bioreactor has been designed to perform under continuous operation at elevated pressure conditions in the production of methanol from methane. All three reactants (water, O₂, and CH₄) are fed through the top-clamping fixture, and then mixed within the bio-lamina plates, which support the biofilm. The products are eluted from the reactor through the top of the top-clamping fixture as illustrated in FIG. 18.

To accomplish the controlled distribution of water and gases throughout the BLP reactor, precisely fabricated microchannels have been implemented to uniformly distribute the water and create mixed gas bubbles within the flowing stream (FIGS. 18 and 19). A shallow micro-venturi-like design has demonstrated reliable use and consistent performance in uniformly distributing liquid from the header region into the immobilized biofilm reactor space. A 6-μm diameter hole was cut into each venture channel to create bubbles containing a CH₄—O₂ mixture necessary to support and react within the biofilm The BLP reactor is placed inside of the experimental test-loop, which has been fabricated and is in the process of initial testing. A high precision HPLC pump is used to deliver the sterilized water from the holding tank to the reactor volume, a HPLC injection loop is placed downstream of the HPLC pump to inject a small volume of sodium carbonate solution for pH adjustments, if additional pH adjustments are required, a small flow of CO₂ can be mixed with the incoming water stream prior to entering the reactor volume. Electronic mass flow controllers are used to control the flow rate of pressurized O₂ and CH₄ into the reactor. FIG. 20 shows the containment box and internal layout of the experimental system.

The reactor pressure is controlled via a backpressure regulator, which maintains desired internal pressure of the BLP reactor. While minimal temperature change is expected throughout the reactor, inlet and outlet liquid temperatures are monitored and recorded throughout the experimental operation. To ensure safe and reliable operation of the BLP reactor safety features are implemented within the experimental system. Solenoid valves are placed on each of the gas streams to terminate reactant gas flow into the system in the event of a power outage and to ensure that gas flow does not start automatically once power is restored.

A pressure relief valve ensures that reactor volume does not operate at pressures exceeding the design values. In the event of a leak of O₂ and CH₄ from the reactor or tubing line, the containment box is under positive N₂ gas pressure to supply constant purging and dilution of gas below flammability limits. All collection vessels and waste streams are diluted with N₂ prior to being released into the hood.

Preliminary experiments using the first generation BLP reactor has led the team to create and implement design and manufacturing changes to help improve reactor production and performance Due to the robust and secure design of the clamping plates the Generation-2 BLP clamping plates is being fabricated from aluminum instead of SS. Aluminum plates enable the ability to quickly and inexpensively fabricate additional plates.

A layer of biofilm was loaded onto the surface of the bottom BLP bio-plate. The biofilm was immobilized to the surface and experiments were executed to determine the effectiveness of the immobilized film and the impact it may have on the fluid flow through the system. Operation of the BLP microreactor proceeded as expected throughout the initial experimental operation, indicating that the presence of the biofilm inside of the reactor is completely compatible, as designed, with multi-phase flow. Post-experimental analysis indicated significant adhesion between the biofilm and the reactor plate surface. As a result, the Generation-2 bio-lamina plates will include a minimal number of pins on the lower plate; thus, increasing the volume of the immobilized biofilm inside of the reactor while providing adequate structural and fluidic support.

### Example 8

In this example, a bio-lamina bioreactor embodiment was evaluated for periods up to 8 days in duration. The buffer strength of the reactor feed and cell suspension used in gel formulation was increased ten-fold to compensate for the high rates of carbon dioxide production and subsequent pH depression within the biofilm caused by methane oxidation in the absence of cyclopropanol inhibition. Combined with minor modifications to the internal gelation procedure described herein, the resulting biofilms were able to withstand continuous flow operation for periods exceeding one week. Observation of the biofilm after one week of operation showed some signs of wear, but the gel was remarkably intact and indicated strong MMO activity throughout the biofilm when tested by naphthalene oxidation assay.

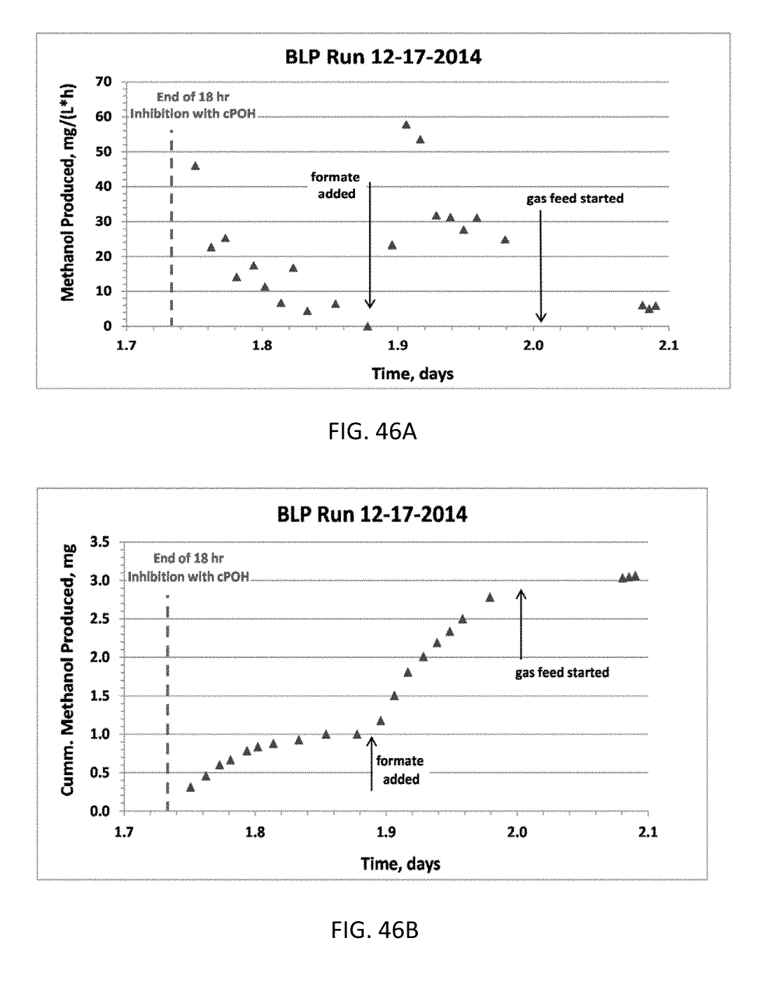

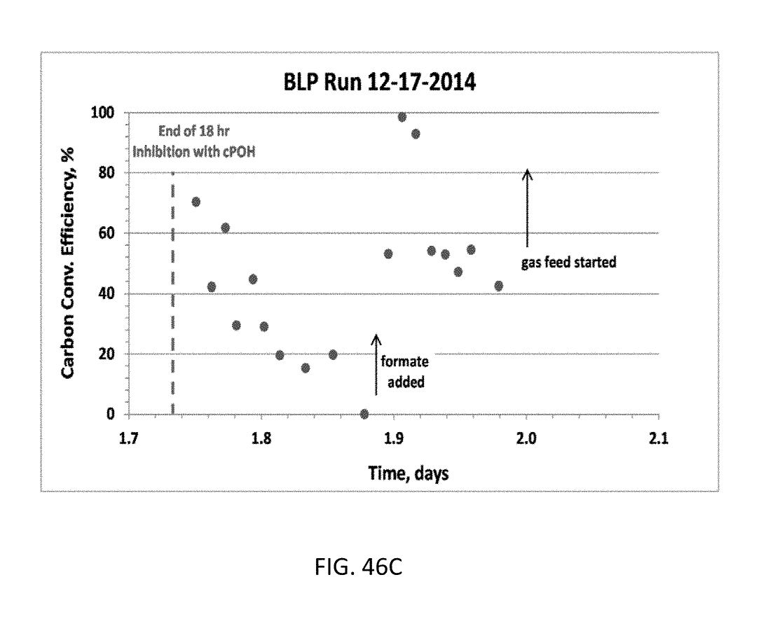

Additionally, methanol production rates exhibited by the bio-lamina bioreactor were up to 4 times greater with more than twice the total amount of methanol produced. Better understanding of the inhibition process has provided the ability to produce more methanol while requiring less cyclopropanol. It has been observed that cyclopropanol inhibition of MDH results in a restriction on re-supply of reducing power to MMO to enable continued high rates of methane oxidation. Addition of exogenous formate to the bio-lamina bioreactor feed after methanol production was observed to cease was shown to result in a burst of methanol production that exceeded that produced from the initial inhibition event itself (FIGS. 46A-46C).

In one example, methane and oxygen addition as separate gas streams to the bio-lamina bioreactor instead of as dissolved gases in the feed solution was successfully achieved and resulted in additional amounts of methanol production after apparent cessation of the process in liquid-feed mode. The additional methanol production was believed to be a result of enhanced methane oxidation rates while methanol oxidation rates remained unchanged.

The examples described above can be used to determine optimal cPOH concentrations and exposure time to maximize MeOH production. In some examples, cPOH inhibition is rapid and irreversible. In some examples, cPOH inhibited MDH of culture immobilized in alginate beads in less than 100 seconds, and MDH inhibition was maintained after alginate beads were rinsed to remove cPOH. In some examples, it was determined that cPOH is a more effective inhibitor of MDH in the absence of MeOH, suggesting the need to inhibit intermittently.

Multiple examples using the bio-lamina bioreactors described herein were evaluated and resulted in modification of operation conditions and gel-biofilm formulation to produce stable reactor performance for periods up to 8 days in duration. In some examples, the buffer strength of the reactor feed and cell suspension used in gel formulation was increased ten-fold to compensate for the high rates of carbon dioxide production and subsequent pH depression within the biofilm caused by methane oxidation in the absence of cyclopropanol inhibition. Combined with minor modifications to the internal gelation procedure, the resulting gels were able to withstand continuous flow operation for periods exceeding one week. Observation of the biofilm after one week of operation showed some signs of wear, but the gel was remarkably intact and indicated strong MMO activity throughout the biofilm when tested by naphthalene oxidation assay.

In some examples, fluxes and uptake profiles of dissolved oxygen from the bio-lamina bioreactor experiments can be evaluated. In some embodiments, a microsensor apparatus for making oxygen and pH gradient measurements in the biofilms at open atmospheric conditions can be used. Dissolved oxygen (DO) and pH microelectrodes with tip diameters of 8-12 μm (Unisense AS, Denmark) are used to take vertical concentration profiles within the biofilm samples. The O₂ sensors are Clark-type microelectrodes. A two-point calibration is performed for DO sensors using medium at atmospheric saturation of DO and medium sparged with pure N_{2(g)} for a zero measurement. The pH microelectrode is calibrated with buffered solutions at pH 4.0, 7.0 and 10.0. The pH microelectrode consisted of a redox sensitive tip 150 μm in length, which measured the pH over the depth of the tip.

Substantial information on the effectiveness of biocatalysts within the biofilm can be gained by probing the spatial distribution of dissolved oxygen and pH within the bio-lamina bioreactor. In some examples, surface-immobilized indicators sensitive to pH and redox potential, which provide colorimetric or fluorescent signals dependent upon local microenvironment can be used. This method can be implemented by installing a small window in the bio-lamina bioreactor.

Colorimetric or fluorescent indicators (e.g. derivatives of fluorescein, bromocresol green and/or cresol red) are immobilized on fine-mesh silica gel. A thin layer of the indicator-modified particles is spread onto a bio-lamina substrate, or (for method development) glass or plastic substrate. The indicator layer is covered with a cell-loaded alginate gel. After operation of the bio-lamina bioreactor (or exposure to the CH₄/O₂ in solution for method development), the bio-lamina substrate can be observed and immediately photographed using a high-resolution camera. The local pH can be assessed across the area of the plate by automated analysis of the resulting color image using NIH ImageJ processing software. A similar technique based on immobilized redox dyes (e.g., resazurin derivatives) can be used to measure the oxygen potential across the reactor plate and within the gel.

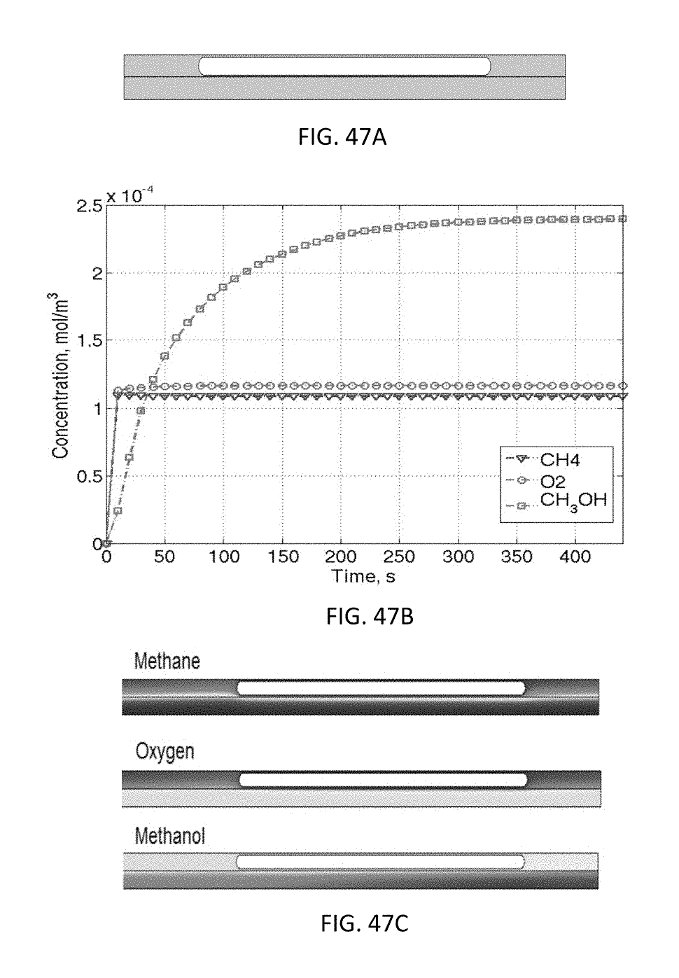

A mathematical model that fully represents the structure, the microbial culture (OB3b) and operating conditions (20 bar O₂, CH₄) of the bio-lamina bioreactor also can be produced, as can a numerical simulation code using COMSOL software to run the mathematical model. Motivated by extremely long computing run-times a number of simplifications are implemented in the model. Simplification consists of modeling only first several cm of reactor length and then extrapolating methanol and reactants flux values to the reactor exit located at 22 cm. Model consists of 1 cm segments (FIGS. 47A-47C) and the number of segments included in the computation can be anywhere between 1 and 22. The structure of model is completely based on the first principles; thus once the model and numerical simulation is verified it could be used in the design of scaled versions of the bio-lamina bioreactor. The parameters of the model and microbial kinetics can be derived from data disclosed herein, calculations, or can be taken from the art.

The simulation results confirmed a long-standing conjecture that a fully enhanced operating conditions of the bio-lamina bioreactor will provide major process intensification goals pertinent to the REMOTE program (greater than 3 [molCH₄/L_{reactor}/hr]. The results emerging from the simulations provide the peak values in the mass transfer characteristics of the bio-lamina bioreactor (depending on the operating conditions) in the range 2.5-8.5 [molCH₄/L_{reactor}/hr] and 1.5-3.5 [mol O₂/L_{reactor}/hr]; which matches the expectations for CH₄ and O₂ fluxes. The equivalent concentrations of CH₄ and O₂ in the liquid phase just above the biofilm reach the maximum saturation point (at 20 bar total pressure) at a very short distance from the reactor entrance; thus, providing immediately the most optimal conditions for the transport into the biofilm. Modeling of only 1 segment takes 14 hours of computational time to reach the steady state methanol production. Due to non-linear computational load scaling, modeling of the whole system with 22 segments is expected to take about a month. An extrapolation procedure has been developed and verified, which provides accurate methanol flux at the outlet of a 10 segments. Methanol molar flux is evaluated through integration at the end of each segment. It increases linearly with reactor length making the fit simple and predictable.

### Example 9

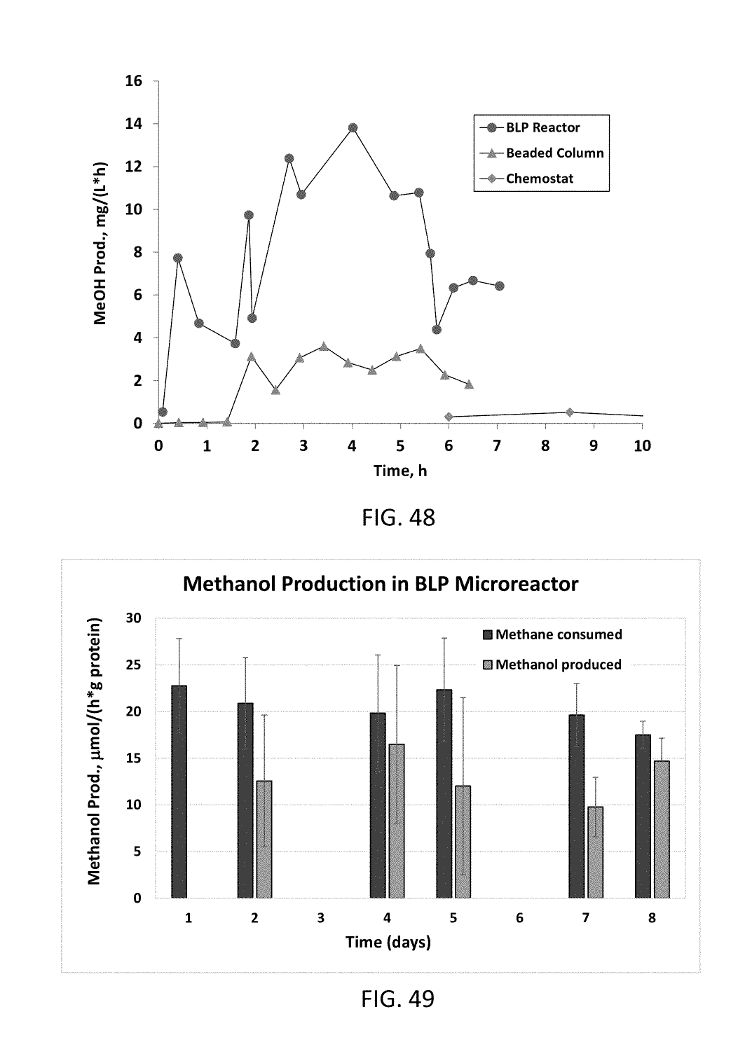

In one example, the performance of a representative bio-lamina bioreactor in methanol production was compared with that of a beaded column comprising an alginate/OB3b matrix and a chemostat comprising OB3b cells. As illustrated in FIG. 48, the micro-scale architecture of the representative bio-lamina bioreactor results in the highest rates of methanol production per volume of reactor, while dispersed growth in the chemostat produced the slowest production rates. Results from the beaded column embodiment are illustrated in FIG. 38, wherein the bars represent the average rates of CH4 consumption and MeOH production on each of the days of active operation of the column. The arrows indicate the periods that cPOH inhibition was applied. Results from the microscale bio-lamina bioreactor are provided by FIG. 49.

### Example 10

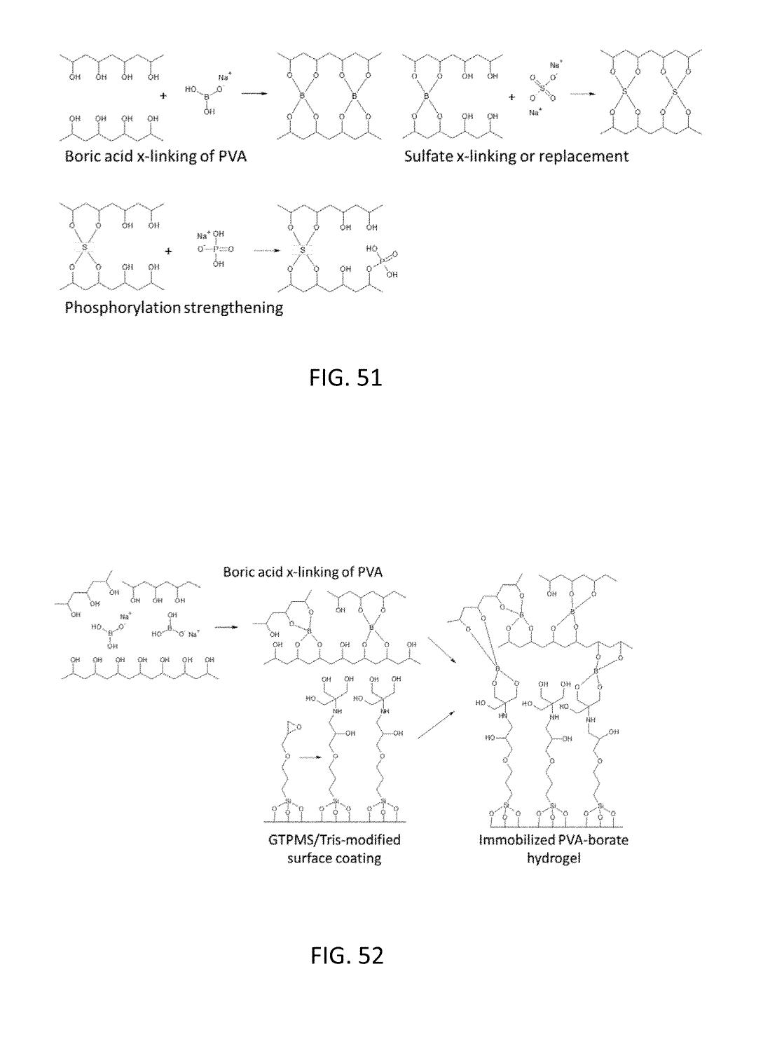

Another example of biofilm preparation can include preparing a bacterial cell slurry by mixing bacterial cells in a 1:1 ratio with 10-20% PVA in water. The gel is then cross-linked by immersion in a solution of boric acid or sodium borate (1-5%) in alkaline buffer (0.5M sodium carbonate). The resulting borate esters are then substituted with more-stable sulfonates by immersion in sodium sulfate (1M). Finally, the gel is strengthened by reaction of phosphates to form phosphate esters, which increase the hydrophobicity of the PVA and prevent dissolution of the gel. FIG. 51 provides a schematic illustration of this embodiment.

### Example 11

In this example, a biofilm bio-lamina substrate is produced using an initial surface modification step that is used to deposit an activated coating on the substrate prior to addition of the biofilm composition. In one embodiment, a metal oxide biofilm bio-lamina substrate is modified by reacting glycidylpropoxytrimethoxysilane (GPTMS) with the metal oxide biofilm bio-lamina substrate and then adding tris(hydroxymethyl)aminomethane (Tris) to produce a hydroxyl-rich surface coating. The resulting hydroxyl groups of the modified metal oxide biofilm bio-lamina substrate can interact with a biofilm composition, such as one comprising a PVA-borate crosslinked biofilm composition, and thereby covalently immobilize the PVA-borate biofilm composition to the surface-modified metal oxide biofilm bio-lamina substrate. This embodiment is illustrated schematically in FIG. 52.

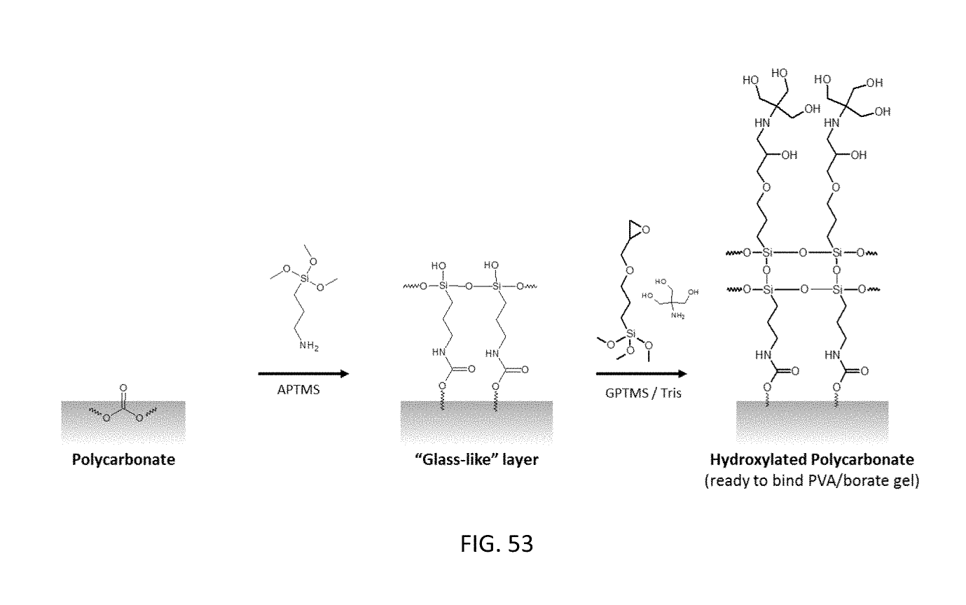

In yet another example, a polycarbonate-based biofilm composition is immobilized. An initial reaction between the carbonate functional groups of polycarbonate and primary amine functional groups of aminopropyltri[m]ethoxysilane (APTMS/APTES) occurs to form a stable carbamate bond. Controlled hydrolysis/crosslinking of the pendant methoxysilane groups forms a thin film of “glass-like” polysiloxane decorated with surface silanol groups. The silanols are then further modified by reaction with GPTMS, forming an epoxy-functionalized surface which is then reacted with the primary amine of tris(hydroxymethyl)aminomethane (Tris). The resulting hydroxylated surface can participate in borate ester formation to immobilize PVA, as discussed above. An exemplary embodiment is illustrated schematically in FIG. 53.

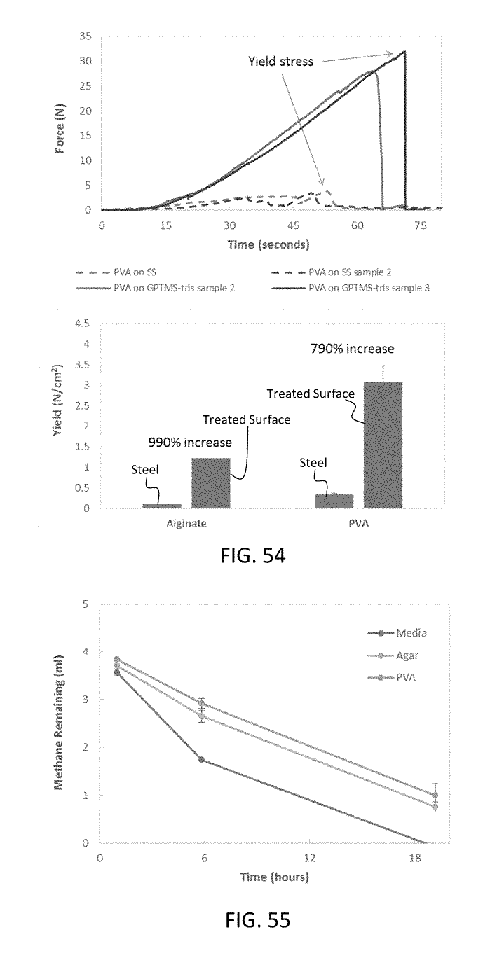

The adhesion strength (lap-shear) of these examples can be tested. In one example, a substrate having a 1 in² contact area and 750 μm thickness (submerged in water) was used as the biofilm bio-lamina substrate with and without surface modification. Results are illustrated in FIG. 54 (top). As illustrated in FIG. 54 (top), PVA adhesion on stainless steel (“SS”) with (solid lines) and without (dashed lines) GPTMS-Tris was evaluated. The yield stress of PVA on the untreated stainless is negligible, but an order-of-magnitude increase in adhesion is observed with an initial surface modification step using GPTMS-Tris. Similar results are shown in FIG. 54 (bottom), which illustrates results for calcium alginate hydrogels on SS with and without aminopropyltrimethoxysilane (APTMS) surface modification treatment.

### Example 12

In another example, methane oxidation behavior of methanotroph, Methylmicrobium buryatense 5G, was evaluated. The Methylmicrobium buryatense 5G activity was evaluated in media, agar, and PVA beads (2-3 mm diameter). Activity in PVA was observed to be only slightly lower than in agar. Activity in agar and PVA were observed to be lower than the freely-suspended cells, likely due to reduced mass-transfer within the relatively large beads (vs. hydrogel films <1 mm thickness in the reactor). Results from this embodiment are illustrated in FIG. 55.

### VII. Overview of Several Embodiments

Disclosed herein are embodiments of a bio-lamina bioreactor, comprising:

a biofilm bio-lamina substrate comprising one or more structural projections;

a biofilm comprising a microorganism, wherein the biofilm is coupled to the bio-lamina substrate; and

a fluid flow bio-lamina substrate comprising one or more structural projections, one or more fluid mixers, one or more feed holes, and one or more channel manifolds.

In some embodiments, the bio-lamina bioreactor further comprise a first clamp plate and a second clamp plate.

In any or all of the above embodiments, the bio-lamina bioreactor can further comprise an inlet for introducing liquid into the bio-lamina bioreactor, an inlet for introducing gas into the bio-lamina bioreactor, and an outlet for delivering fluid from the bio-lamina bioreactor.

In any or all of the above embodiments, the bio-lamina bioreactor comprises two inlets for introducing gas into the bio-lamina bioreactor.

In any or all of the above embodiments, the biofilm bio-lamina substrate and the fluid flow bio-lamina substrate is a polymer substrate comprising polycarbonate, polyethylene terephthalate (PET), polyether imide (PEI), poly(methyl methacrylate) (PMMA), poly(tetrafluoroethylene) (PTFE), or a combination thereof.

In any or all of the above embodiments, the biofilm bio-lamina substrate and the fluid flow bio-lamina substrate is a metal substrate comprising a metal selected from stainless steel, copper, titanium, nickel, aluminum, or combinations thereof.

In any or all of the above embodiments, a surface of the biofilm bio-lamina substrate is surface-modified with glycidylpropoxytrimethoxysilane, tris(hydroxymethyl)aminomethane, aminopropyltriethoxysilane, aminopropyltrimethoxysilane, or combinations thereof.

In any or all of the above embodiments, the biofilm has a thickness of 10 μm to 1 mm.

In any or all of the above embodiments, the biofilm further comprises a film-forming matrix.

In any or all of the above embodiments, the film-forming matrix is formed between a polysaccharide, a polymer, or a combination thereof, and an inorganic salt.

In any or all of the above embodiments, the polysaccharide is alginate and the inorganic salt is CaCl₂.

In any or all of the above embodiments, the polymer is polyvinyl alcohol, hydrolyzed polymaleic anhydride, polyacrylic acid, polycarbonate, or a combination thereof; and the inorganic salt is sodium borate, sodium sulfate, sodium phosphate, or a combination thereof.

In any or all of the above embodiments, the film-forming matrix further comprises polylysine, chitosan, adipic dihydrazide, or an aminosilane.

In any or all of the above embodiments, the microorganism is a methanotroph.

In any or all of the above embodiments, the biofilm comprises a combination of a methanotroph, alginate, and calcium ions.

In any or all of the above embodiments, the biofilm is covalently attached to the biofilm bio-lamina substrate.

In any or all of the above embodiments, the biofilm is covalently attached to the biofilm bio-lamina substrate through the polylysine, chitosan, adipic dihydrazide, or the aminosilane.

In any or all of the above embodiments, the biofilm is electrostatically coupled to the biofilm bio-lamina substrate.

In any or all of the above embodiments, the biofilm bio-lamina substrate and the fluid flow bio-lamina substrate comprise a plurality of structural projections.

In any or all of the above embodiments, the plurality of structural projections present on the fluid flow bio-lamina substrate are configured to provide a gradient through which fluid flows.

In any or all of the above embodiments, the plurality of structural projections comprises structural projections of different sizes to form the gradient.

In any or all of the above embodiments, the one or more fluid mixers comprise elevated projections that are configured to provide a tapered flow channel through which liquid can flow.

In any or all of the above embodiments, the feed hole is located within the tapered flow channel.

In any or all of the above embodiments, the one or more channel manifolds each comprise at least one channel and at least one opening through which gas or liquid can be introduced.

In any or all of the above embodiments, the fluid flow bio-lamina substrate comprises a plurality of fluid mixers and a plurality of channel manifolds.

In some embodiments, devices are described comprising:

a biofilm bio-lamina substrate comprising a plurality of structural projections and wherein the biofilm bio-lamina substrate is coupled to a biofilm comprising a film-forming material and a microorganism embedded in the film-forming material;

a fluid flow bio-lamina substrate comprising a plurality of structural projections configured to align with the plurality of structural projections of the biofilm bio-lamina substrate; a plurality of fluid mixers each comprising a tapered flow channel and a feed hole; a first channel manifold comprising a first opening; and a second channel manifold comprising a second opening;

a top clamp plate comprising a plurality of alignment pins; and

a bottom clamp plate comprising a plurality of alignment holes configured to accept the plurality of alignment pins of the top clamp plate.

Also disclosed herein are embodiments of a biofilm bio-lamina substrate coupled to a biofilm comprising a microorganism, wherein the biofilm bio-lamina substrate comprises one or more structural projections.

In some embodiments, the biofilm bio-lamina substrate is covalently or electrostatically coupled to the biofilm.

Also disclosed herein are embodiments of a fluid flow bio-lamina substrate, comprising one or more structural projections, one or more fluid mixers, and one or more channel manifolds.

In some embodiments, the one or more fluid mixers comprise elevated projections that are configured to provide a tapered flow channel through which liquid can flow.

In any or all of the above embodiments, the fluid flow bio-lamina substrate further comprises a feed hole positioned with the tapered flow channel.

In any or all of the above embodiments, the one or more channel manifolds each comprise at least one channel and at least one opening through which gas or liquid can be introduced.

In any or all of the above embodiments, the fluid flow bio-lamina substrate comprises a plurality of fluid mixers and a plurality of channel manifolds.

Also disclosed herein are embodiments of a method for making a biofilm bio-lamina substrate, comprising:

combining a microorganism cell and a polysaccharide to form a biofilm precursor solution;

covering at least a portion of a top surface of a bio-lamina substrate comprising one or more structural projection with the biofilm precursor solution to form a biofilm precursor layer; and

exposing the biofilm precursor layer to an inorganic salt component to promote crosslinking of the polysaccharide to thereby form a biofilm on the bio-lamina substrate.

In some embodiments, the method can further comprise using an internal gelation system to form the biofilm.

In any or all of the above embodiments, the internal gelation system comprises glucono-delta-lactone, calcium carbonate, calcium sulfate, or combinations thereof.

In any or all of the above embodiments, the method further comprises pre-treating the bio-lamina substrate with an organic polymer or linking agent prior to covering the top surface of the bio-lamina substrate with the biofilm precursor solution.

In any or all of the above embodiments, the method further comprises pre-treating the bio-lamina substrate with polylysine, chitosan, adipic dihydrazide, or an aminosilane.

Also disclosed herein are embodiments of a method, comprising:

introducing a liquid and at least one organic reactant into a bio-lamina bioreactor comprising a biofilm bio-lamina substrate comprising one or more structural projections and coupled to a biofilm comprising a microorganism; a fluid flow bio-lamina substrate comprising one or more structural projections, one or more fluid mixers, and one or more channel manifolds; a top clamp plate; and a bottom clamp plate; and

isolating a fuel produced by reaction of the organic reactants with the microorganism that is expelled from the bio-lamina bioreactor.

In some embodiments, the liquid is water and the at least one organic reactant is a gas.

In any or all of the above embodiments, the gas is selected from methane, oxygen, and combinations thereof.

In any or all of the above embodiments, the liquid is introduced into the bio-lamina bioreactor at a rate of greater than 0 mL/hr to 500 mL/hr and the organic reactant is introduced into the bio-lamina bioreactor at a rate of greater than 0 mL/hr to 5,000 mL/hr at 1 atm.

In any or all of the above embodiments, the method comprises introducing a first organic reactant into the bio-lamina bioreactor and introducing a second organic reactant into the bio-lamina bioreactor.

In any or all of the above embodiments, the first organic reactant and the second organic reactant are introduced into the bio-lamina bioreactor sequentially or simultaneously.

In any or all of the above embodiments, the first organic reactant and the second organic reactant are introduced into the bio-lamina bioreactor as a mixture.

In any or all of the above embodiments, the mixture comprises methane gas and oxygen.

In any or all of the above embodiments, the mixture comprises 1/3 methane gas (v/v) and 2/3 oxygen gas (v/v).

In view of the many possible embodiments to which the principles of the present disclosure may be applied, it should be recognized that the illustrated embodiments are only preferred examples of the present disclosure and should not be taken as limiting the scope of the disclosure. Rather, the scope of the disclosure is defined by the following claims.

We claim:

1. A biofilm bio-lamina substrate, comprising one or more structural projections and a covalently- or electrostatically-coupled biofilm comprising a microorganism.

2. The biofilm bio-lamina substrate of claim 1, further comprising a fluidly-associated fluid flow bio-lamina substrate comprising one or more structural projections, one or more fluid mixers, one or more feed holes, and one or more channel manifolds, wherein the biofilm bio-lamina substrate and the fluidly-coupled fluid flow bio-lamina substrate form a bio-lamina bioreactor.

3. The biofilm bio-lamina substrate of claim 2, further comprising:
a first clamp plate;
a second clamp plate;
an inlet for introducing liquid into the bio-lamina bioreactor;
an inlet for introducing gas into the bio-lamina bioreactor;
an outlet for delivering fluid from the bio-lamina bioreactor; or
any combination thereof.

4. The biofilm bio-lamina substrate of claim 2, wherein each of the biofilm bio-lamina substrate and the fluidly-coupled fluid flow bio-lamina substrate independently comprises a polymer substrate comprising polycarbonate, polyethylene terephthalate (PET), polyether imide (PEI), poly(methyl methacrylate) (PMMA), poly(tetrafluoroethylene) (PTFE), or a combination thereof; or a metal substrate comprising a metal selected from stainless steel, copper, titanium, nickel, aluminum, or combinations thereof.

5. The biofilm bio-lamina substrate of claim 1, wherein the biofilm bio-lamina substrate comprises a surface that is surface-modified with glycidylpropoxytrimethoxysilane, tris(hydroxymethyl)aminomethane, aminopropyltriethoxysilane, aminopropyltrimethoxysilane, or combinations thereof.

6. The biofilm bio-lamina substrate of claim 1, wherein the biofilm further comprises a film-forming matrix comprising a polysaccharide, a polymer, or a combination thereof, and an inorganic salt.

7. The biofilm bio-lamina substrate of claim 6, wherein (i) the polysaccharide is alginate and the inorganic salt is CaCl₂; or (ii) the polymer is polyvinyl alcohol, hydrolyzed polymaleic anhydride, polyacrylic acid, polycarbonate, or a combination thereof; and the inorganic salt is sodium borate, sodium sulfate, sodium phosphate, or a combination thereof.

8. The biofilm bio-lamina substrate of claim 6, wherein the film-forming matrix further comprises polylysine, chitosan, adipic dihydrazide, or an aminosilane.

9. The biofilm bio-lamina substrate of claim 1, wherein the biofilm comprises a methanotroph, alginate, and calcium ions.

10. The biofilm bio-lamina substrate of claim 1, wherein the biofilm is covalently attached to the biofilm bio-lamina substrate or electrostatically coupled to the biofilm bio-lamina substrate.

11. The biofilm bio-lamina substrate of claim 8, wherein the biofilm is covalently attached to the biofilm bio-lamina substrate through the polylysine, chitosan, adipic dihydrazide, or aminosilane.

12. The biofilm bio-lamina substrate of claim 2, wherein the biofilm bio-lamina substrate and the fluid flow bio-lamina substrate comprise a plurality of structural projections having different sizes, which are configured to provide a gradient through which fluid flows.

13. The biofilm bio-lamina substrate of claim 2, wherein the one or more fluid mixers comprise elevated projections that are configured to provide a tapered flow channel through which liquid can flow, and wherein the tapered flow channel comprises a feed hole.

14. The biofilm bio-lamina substrate of claim 2, wherein the one or more channel manifolds each comprise at least one channel and at least one opening through which gas or liquid can be introduced.

15. The biofilm bio-lamina substrate of claim 3, comprising:
the top clamp plate, the top clamp plate comprising a plurality of alignment pins;
the bottom clamp plate, the bottom clamp plate comprising a plurality of alignment holes configured to accept the plurality of alignment pins of the top clamp plate;
the biofilm bio-lamina substrate, the biofilm bio-lamina substrate comprising a plurality of structural projection;
the biofilm, the biofilm comprising a film-forming material in which the microorganism is embedded; and
the fluidly-associated fluid flow bio-lamina substrate, the fluidly-associated fluid flow bio-lamina substrate comprising (i) a plurality of structural projections configured to align with the plurality of structural projections of the biofilm bio-lamina substrate; (ii) a plurality of fluid mixers each comprising a tapered flow channel and a feed hole; (iii) a first channel manifold comprising a first opening; and (iv) a second channel manifold comprising a second opening.

16. A fluid flow bio-lamina substrate, comprising one or more structural projections, one or more fluid mixers comprising elevated projections that are configured to provide a tapered flow channel through which liquid can flow and wherein the tapered flow channel comprises a feed hole, and one or more channel manifolds comprising at least one channel and at least one opening through which gas or liquid can be introduced.

17. A bio-lamina bioreactor, comprising:
the biofilm bio-lamina substrate and the fluidly-coupled fluid flow bio-lamina substrate of claim 2;
a first clamp plate;
a second clamp plate;
an inlet for introducing liquid into the bio-lamina bioreactor;
an inlet for introducing gas into the bio-lamina bioreactor; and
an outlet for delivering fluid from the bio-lamina bioreactor.

18. A method for making the biofilm bio-lamina substrate of claim 1, comprising:
combining a microorganism cell and a polysaccharide to form a biofilm precursor solution;
covering at least a portion of a top surface of the bio-lamina substrate with the biofilm precursor solution to form a biofilm precursor layer; and
exposing the biofilm precursor layer to an inorganic salt component to promote crosslinking of the polysaccharide to thereby form the biofilm on the bio-lamina substrate.

19. The method of claim 17, further comprising using an internal gelation system comprising glucono-delta-lactone, calcium carbonate, calcium sulfate, or combinations thereof to form the biofilm.

20. The method of claim 17, wherein the method further comprises pre-treating the bio-lamina substrate with an organic polymer or linking agent prior to covering the top surface of the bio-lamina substrate with the biofilm precursor solution.

21. A method, comprising:
introducing a liquid and at least one organic reactant into the bio-lamina bioreactor of claim 17; and
using the bio-lamina bioreactor.

22. The method of claim 21, wherein using comprises isolating a fuel produced by reaction of the at least one organic reactant with the microorganism that is expelled from the bio-lamina bioreactor.

23. The method of claim 21, wherein the liquid is water and the at least one organic reactant is a gas selected from methane, oxygen, and combinations thereof and wherein the liquid is introduced into the bio-lamina bioreactor at a rate of greater than 0 mL/hr to 500 mL/hr and the organic reactant is introduced into the bio-lamina bioreactor at a rate of greater than 0 mL/hr to 5,000 mL/hr at 1 atm.

24. The method of claim 21, wherein the method comprises introducing a first organic reactant into the bio-lamina bioreactor and introducing a second organic reactant into the bio-lamina bioreactor, wherein the first organic reactant and the second organic reactant are introduced into the bio-lamina bioreactor sequentially or simultaneously, or wherein the first organic reactant and the second organic reactant are introduced into the bio-lamina reactor as a mixture.
